(19) United States
(12) Patent Application Publication
(10) Pub. No.: US 2004/0072261 A1
(43) Pub. Date: Apr. 15, 2004

(54) Method for the diagnosis and differential diagnosis of neurological diseases

(76) Inventors: Vesna Kostanjevecki, Sint-Denijs-Westrem (BE); Eugeen Vanmechelen, Nazareth-Eke (BE); Veronique De Brabandere, Gent (BE)

Correspondence Address: Patricia A. Kammerer, HOWREY SIMON ARNOLD & WHITE, LLP, 750 Bering Drive, Houston, TX, 77057-2198, US

(21) Appl. No.: 10/601,100

(22) Filed: Jun. 20, 2003

Related U.S. Application Data
(60) Provisional application No. 60/396,438, filed on Jul. 17, 2002.

(30) Foreign Application Priority Data
Jun. 21, 2002 (EP) 02447121.1

(51) Int. Cl. G01N 33/53; G01N 33/567

(52) U.S. Cl. 435/7.2

(57) ABSTRACT

A method is provided for the screening, diagnosis and/or prognosis of neurological diseases. More specifically, new biomarkers are provided for the screening, diagnosis and/or prognosis in a mammal of Alzheimer's disease, frontotemporal dementia, dementia with Lewy bodies, vascular dementia and/or depression. The method further provides for the differential diagnosis in a mammal of Alzheimer's disease, frontotemporal dementia, dementia with Lewy bodies, vascular dementia and/or depression.

This application claims priority to EP 02447121.1 filed Jun. 21, 2002 and U.S. provisional application No. 60/396,438 filed Jul. 17, 2002, the entire contents of which are incorporated by reference herein.

### FIELD OF THE INVENTION

The present invention relates to the diagnosis and differential diagnosis of neurological diseases. More specifically, the present invention provides new biomarkers for the screening, diagnosis or prognosis of Alzheimer's disease, frontotemporal dementia, dementia with Lewy bodies, vascular dementia and/or depression. The present invention further provides new biomarkers for the differential diagnosis of Alzheimer's disease, frontotemporal dementia, dementia with Lewy bodies, vascular dementia and/or depression.

### BACKGROUND ART

Dementia is a serious, common, and rapidly growing worldwide problem associated with increased healthcare utilization. It is a major predictor of morbidity and mortality in the elderly. The occurrence of the more than 100 known diseases that produce this condition depends on age, as well as genetic factors linked to geography, race, and ethnicity. Dementia can be defined as a chronic deterioration in multiple cognitive abilities (memory, attention, judgment, etc.) that impairs the previously successful performance of activities of daily living. Its clinical profile and degree of severity are affected not only by the total quantity of neuronal loss, but by the specific locations of the underlying lesions. By far the most common forms of dementia are Alzheimer's disease (40-60% of the cases), dementia with Lewy bodies (10-20% of the cases), vascular dementia (25% and possibly contributing in up to 40% of the cases), and frontotemporal dementia (for which prevalence remains unclear) (Lowe, 2001; Leys et al., 2002; McKeith, 2002; Knopman et al., 2002). More than 33% of women and 20% of men over the age of 65 will develop dementia or milder forms of cognitive impairment in their lifetime (Yaffe and Gregg, 2002). Alzheimer's disease (AD), the principle form and prototype of dementia, may be classified according to different criteria. From the genetic point of view, the disease can be categorized into two types: (i) less frequent, inherited familial forms (ranging from <5% for early-onset to 10-15% for late-onset forms when all genetic predisposition factors are included), and (ii) the far more common sporadic type for which no obvious inheritance patterns have been established. The sporadic form generally emerges after 65 years of age, and is thought to be multifactorial in nature. The definitive diagnosis of AD is based on the finding of disruptively large amounts of senile plaques and neurofibrillary tangles in the affected areas of the neocortex at autopsy. Along with massive gray matter atrophy, these two types of abnormal structures are the hallmarks of the disease.

The second most common cause of primary dementia, after Alzheimer's disease, is dementia with Lewy bodies (DLB) accounting for no less than 10% to 20% of all cases of dementia (Lowe, 2001; McKeith, 2002). The disease-defining Lewy bodies are neuronal inclusions composed of abnormally phosphorylated neurofilaments, ubiquitin, and alpha-synuclein. These abnormalities are thought to contribute to neurological dysfunction resulting in clinical symptoms which, depending on the brain region affected, may partially resemble those associated with Alzheimer's and Parkinson's disease. Indeed, many cases of DLB are still erroneously misdiagnosed as Alzheimer's disease. However, differentiation of DLB from Alzheimer's disease is important. This is because certain neuroleptic agents, extensively prescribed for the psychotic symptoms and behavioral disturbances common in dementia, may result in severe (potentially lethal) hypersensitivity reactions in the case of DLB (Mc Keith, 2002). In addition, the pathological mechanisms that cause DLB may be fundamentally different from those in AD and, accordingly, the differentiation of DLB from AD might be of relevance for disease-modifying treatment aimed at these pathological mechanisms.

Vascular dementia (VAD) is also encountered commonly in older patients. It has been suggested that the cumulative effects of multiple episodes of cerebral ischemia (discrete cerebral strokes) cause the cognitive decline that occurs in many patients. However, at present, the true frequency of pure vascular dementia remains uncertain (Qui et al., 2002), partly because of a lack of consensus on clinical and neuropathological criteria, and because of its clinical resemblance to other dementias.

It is thought that both the prevalence and incidence rates of vascular dementia increase with age, but less rapidly than in Alzheimer's disease. In many cases, histological examination reveals co-morbidity with Alzheimer's disease or DLB, thereby warranting a diagnosis of mixed dementia (Lowe, 2001; Qui et al., 2002). Sometimes VAD is difficult to distinguish from AD and/or depression (Alagiakrishnan and Masaki, 2001). As some vascular problems can be treated in VAD, an early and correct diagnosis of VAD is, however, crucial.

For its part, frontotemporal dementia (FTD) is a focal form of dementia resulting from progressive atrophy of the frontal and temporal lobes of the brain. In its early stages, it leads to profound disturbances in character, socially disruptive behavior, altered reasoning, and impaired ‘executive function’. The latter term refers to the central organizing function of the brain that permits systematic, goal-directed activities involved with planning, organizing, and initiating actions, or with changing behavior or plans when necessary. The onset of frontotemporal dementia most commonly occurs between the ages of 45 and 65 years, i.e., somewhat earlier than Alzheimer's disease. Mutations in the tau gene account for some of the familial cases linked to the disease. Despite its relative frequency, frontotemporal dementia remains poorly recognized due to the heterogeneity of its clinical presentation, histological patterning, and topical distribution in the frontal and temporal brain lobes (Snowden et al., 2002). The current treatment for AD patients, acetylcholinesterase inhibitors, is not effective in FTD patients (Moghul and Wilkinson, 2001). A correct differential diagnosis between AD and FTD is therefore crucial.

In contrast to dementia disorders, a depressive disorder is an illness that involves the body, mood, and thoughts. It affects the way a person eats and sleeps, the way someone feels about himself/herself and the way he/she thinks. The underlying pathophysiology of major depressive disorder (MDD) is not well-defined. Clinical and preclinical trials suggest a disturbance in CNS serotonin activity as an important factor. In the U.S., lifetime incidence of MDD is 20% in women and 12% in men (Aronson, 2002). As depression can be treated, it is important to diagnose depression correctly and to clearly differentiate depression from dementia.

Most neurological conditions for which the patient seeks general medical care can be identified by a combination of different investigations. Diagnosis of dementias such as AD, is currently based on a broad, comprehensive work-up that consists of (i) a thorough clinical evaluation (incl. physical exam, anamnesis with patient and family, medication review); (ii) a neurological examination involving neuropsychological tests and radiology; and (iii) laboratory testing (e.g., vitamin B12, folic acid, thyroid function, complete blood chemistry and blood count, etc.) (Marin et al., 2002) and exclusion of all other forms of dementia. However, ultimately, only confirmation by autopsy can unequivocally differentiate between the various dementing disorders. Some techniques for diagnosis of neurological diseases in patients have been developed such as positron emission tomography (PET), single photon emission computed tomography (SPECT) and nuclear magnetic resonance spectroscopy (NMRS), making it possible to study brain function and structure. Most neurological diseases, however, are still only diagnosed clinically. Clinical evaluation of neurological diseases is complex, as the physician must rule out other problems or disorders that exhibit similar symptoms. Only after accurate diagnosis an effective management and treatment of the disease possible.

In view of the discovery of disease-modifying compounds, which are likely to have their maximum benefit in the early stages of disease and well before neurodegeneration is widespread, there is a great need for reliable early diagnosis of AD and other neurological diseases, and for an accurate differential diagnosis between neurological diseases. Biochemical diagnostic markers (biomarkers), which reflect the pathogenic processes in the brain, can add to the accuracy of this early and differential diagnosis. A number of candidate biomarkers for neurological diseases have been identified. Lütjohann et al. (2000), for example, noted a slight increase in 24S-hydroxycholesterol in plasma of AD and VAD patients compared to the level in healthy controls and depressed patients. Montine et al. (1998 and 2000) report on increased concentrations of prostaglandin E2 and F2-isoprostanes and decreased concentrations of 6-keto-PGF1α in AD patients. The light subtype of the neurofilament protein was increased in AD patients compared with controls (Sjögren et al. 2001). Several CSF proteins, analyzed by 2-dimensional electrophoresis, have been suggested as diagnostic markers for degenerative disorders. Examples are 14-3-3 brain protein, p130 and p131 as markers for Creutzfeldt-Jacob disease (Zerr et al., 1996; Hsich et al., 1996), and the middle isoform of α-2 haptoglobulin for Alzheimer's disease and schizophrenia (Johnson et al., 1992). Levels of glutamine synthetase were significantly increased in CSF from patients with AD and to a lesser extent in patients with VAD (Tumani et al. 1999). CSF-phospho-tau levels were increased in AD patients compared with age-matched controls, while decreased in patients with FTD (Vanmechelen et al. 2000). Phospho-tau was also shown to be a good marker for the differential diagnosis of AD versus DLB and AD versus FTD (International patent application published under WO 02/03073). Combined measurements of β-amyloid and tau in CSF have become a valuable diagnostic tool during recent years, predicting more than 80% of AD cases (Andreasen et al. 1999; 2001; Sunderland et al., 2003).

It is also accepted that Apo E is involved in the transport of lipids to brain cells as well as in the clearance of excess lipids and β-amyloid from plaques in the brain (Wolozin, 2001). As the brain levels of Apo E are increased in neurodegeneration, one would expect increased Apo E levels in the CSF as well. For AD patients, however, contradictory results have been reported for the Apo E levels in CSF (Blennow et al., 1994; Landen et al., 1996; Skoog et al., 1997; Molina et al., 1999; Hesse et al., 2000; Demeester et al., 2000; Fukuyama et al., 2000; Rosler et al., 2001; Csernansky et al., 2002; Davidsson et al., 2002), while a decreased serum Apo E concentration was reported (Slooter et al., 1998; Siest et al., 2000) and an increased plasma Apo E concentration (Taddei et al., 1997). In patients suffering VAD, Apo E concentrations in the CSF were decreased (Landen et al., 1996; Skoog et al., 1997), while studies on the Apo E levels in CSF from patients suffering FTD have been contradictory (Blennow et al., 1994; Landen et al., 1996; Molina et al., 1999). None of these studies differentiates Apo E proteins with a different molecular weight or a truncation.

In order to further increase the predictability of AD, especially early in the course of the disease, to improve the diagnosis for other neurological diseases, to be able to differentiate between dementing and non-dementing disorders such as depression, and to provide for the differential diagnosis between neurodegenerative diseases, there is a substantial need to find additional new, complementary disease markers. Novel biochemical markers and their possible combination with previously established biochemical and genetic markers could further strengthen diagnosis and provide useful information for treatment.

### SUMMARY OF THE INVENTION

The present invention provides a method for the screening, diagnosis and/or prognosis in a mammal of one or more neurological diseases, for identifying a mammal at risk of developing one or more neurological diseases or for monitoring the effect of therapy administered to a mammal having one or more neurological diseases.

More specifically, the present invention provides a method for the screening, diagnosis and/or prognosis in a mammal of Alzheimer's disease, frontotemporal dementia, dementia with Lewy bodies, vascular dementia and/or depression.

The present invention provides a method for identifying a mammal at risk of developing Alzheimer's disease, frontotemporal dementia, dementia with Lewy bodies, vascular dementia and/or depression.

The present invention provides a method for monitoring the effect of therapy administered to a mammal having Alzheimer's disease, frontotemporal dementia, dementia with Lewy bodies, vascular dementia and/or depression.

The present invention also provides a method for the differential diagnosis in a mammal of different neurological diseases, among which are Alzheimer's disease, frontotemporal dementia, dementia with Lewy bodies, vascular dementia and/or depression.

The methods of the invention comprise the following steps:

(a) detecting, in said mammal, the level of at least one of the following proteins: Apo E, α-1-antitrypsin, α-1-β glycoprotein, antithrombin III, Apo A-I, Apo A-IV, Apo J, gelsolin, haptoglobin, hemopexin, Ig α-1 chain C region (heavy), kininogen, prostaglandin-H2 D-isomerase, transthyretin, vitamin D-binding protein, Zn-α-2-glycoprotein, or of an isoform thereof; and

(b) comparing the level of said at least one protein or protein isoform detected in step (a) with a range of levels of said at least one protein or protein isoform previously defined as characteristic for mammals suffering from AD, with a range of levels of said at least one protein or protein isoform previously defined as characteristic for mammals suffering from FTD, with a range of levels of said at least one protein or protein isoform previously defined as characteristic for mammals suffering from DLB, with a range of levels of said at least one protein or protein isoform previously defined as characteristic for mammals suffering from VAD, with a range of levels of said at least one protein or protein isoform previously defined as characteristic for mammals suffering from depression and with a range of levels of said at least one protein or protein isoform previously defined as characteristic for control mammals; and

(c) concluding from the comparison in step (b) whether the mammal is suffering from Alzheimer's disease, frontotemporal dementia, dementia with Lewy bodies, vascular dementia and/or depression, whereby a level of said at least one protein or protein isoform in a range previously defined as characteristic for mammals suffering from AD is an indication that said mammal is suffering from AD; and whereby a level of said at least one protein or protein isoform in a range previously defined as characteristic for mammals suffering from FTD is an indication that said mammal is suffering from FTD; and whereby a level of said at least one protein or protein isoform in a range previously defined as characteristic for mammals suffering from DLB is an indication that said mammal is suffering from DLB; and whereby a level of said at least one protein or protein isoform in a range previously defined as characteristic for mammals suffering from VAD is an indication that said mammal is suffering from VAD; and whereby a level of said at least one protein or protein isoform in a range previously defined as characteristic for mammals suffering from depression is an indication that said mammal is suffering from depression.

More particularly, the methods of the invention comprise the following steps:

(a) detecting, in said mammal, the level of at least one of the following proteins: Apo E, α-1-antitrypsin, α-1-β glycoprotein, antithrombin III, Apo A-I, Apo A-IV, Apo J, gelsolin, haptoglobin, hemopexin, Ig α-1 chain C region (heavy), kininogen, prostaglandin-H2 D-isomerase, transthyretin, vitamin D-binding protein, Zn-α-2-glycoprotein, or of an isoform thereof; and

(b) comparing the level of said at least one protein or protein isoform detected in step (a) with the level of said at least one protein or protein isoform in a control mammal or in a mammal suffering from another neurological disease; and

(c) concluding from the comparison in step (b) whether the mammal is suffering from Alzheimer's disease, dementia with Lewy bodies, frontotemporal dementia, vascular dementia and/or depression.

The present invention further provides protein isoforms that are associated with one or more neurological diseases, among which are Alzheimer's disease, frontotemporal dementia, dementia with Lewy bodies, vascular dementia and/or depression.

The present invention also provides a composition comprising at least one of the above protein isoforms in isolated form.

The present invention further provides antibodies that specifically recognize the protein isoforms of the invention.

The present invention further provides a kit for the screening, diagnosis and/or prognosis in a mammal of Alzheimer's disease, frontotemporal dementia, dementia with Lewy bodies, vascular dementia and/or depression.

The present inventions also provides a kit for identifying a mammal at risk of developing Alzheimer's disease, frontotemporal dementia, dementia with Lewy bodies, vascular dementia and/or depression.

The present invention also provides a kit for monitoring the effect of therapy administered to a mammal having Alzheimer's disease, frontotemporal dementia, dementia with Lewy bodies, vascular dementia and/or depression.

The present invention further provides a kit for the differential diagnosis in a mammal of Alzheimer's disease, frontotemporal dementia, dementia with Lewy bodies, vascular dementia and/or depression.

The present invention further provides a method of screening for agents that interact and/or modulate the expression or activity of a protein isoform of the invention, associated with one or more neurological diseases, among which are Alzheimer's disease, frontotemporal dementia, dementia with Lewy bodies, vascular dementia and/or depression.

### FIGURE LEGENDS

FIG. 1. Digital CSF 2-D master map. All annotated spots were identified by MS sequencing and were differentially expressed (p<0.05) between AD1-6, FTD1-6, and controls C1-6 CSF samples as listed in Table 2.

FIG. 2. Example of a 2-D gel image. All annotated spots were identified by MS sequencing and were altered (p<0.05) when comparing AD7-12 with D1-6 CSF samples. The gel was loaded with 600-μl depleted CSF obtained from a patient with depression (D6). The enlarged section in the upper right corner (2a) shows details of Apo A-I spots, and the section in the lower right corner (2b) shows the identified Apo E spots.

FIG. 3. (3a) Relation of Apo A-I isoform expression between analyzed groups: 6 AD (AD 1-6), 10 FTD (FTD 1-6, B 3-4 and B 7-8) and 4 VAD (B 1-2 and B 5-6). The mean isoform intensity of the AD group was equated at 100% with intensities in other groups expressed in relation to the AD group. (3b) Relation of Apo A-I isoform expression between analyzed groups: 6 AD (AD 7-12) and 6 D (D 1-6). The mean isoform intensity of the AD group was equated at 100% with intensities in other groups expressed in relation to the AD group.

FIG. 4. Example of a 2-D gel image obtained when comparing AD 7-12 with D 1-6 CSF samples. The gel was loaded with 600-μl depleted CSF obtained from a patient with depression (D 6). The Apo E isoforms of the invention are indicated.

FIG. 5. Immunoblot (anti-Apo E antibody 31F4B5) of a 2-D gel obtained with a CSF sample pool. NPI 60, 73, 74 and 75 were matched with the 2-D gel of FIG. 4.

FIG. 6. Colloidal gold staining of the immunoblot of FIG. 5.

### DETAILED DESCRIPTION OF THE INVENTION

The present invention relates to methods for the screening, (differential) diagnosis and/or prognosis in a mammal of one or more neurological diseases, among which are Alzheimer's disease (AD), frontotemporal dementia (FTD), dementia with Lewy bodies (DLB), vascular dementia (VAD) and/or depression (D), to a method for identifying a mammal at risk of developing one or more neurological diseases, among which are Alzheimer's disease, frontotemporal dementia, dementia with Lewy bodies, vascular dementia and/or depression, or to a method for monitoring the effect of a therapy administered to a mammal having one or more neurological diseases, among which Alzheimer's disease, frontotemporal dementia, dementia with Lewy bodies, vascular dementia and/or depression. The methods of the invention comprise the following steps:

(a) detecting, in said mammal, the level of at least one of the following proteins: Apo E, α-1-antitrypsin, α-1-β glycoprotein, antithrombin III, Apo A-I, Apo A-IV, Apo J, gelsolin, haptoglobin, hemopexin, Ig α-1 chain C region (heavy), kininogen, prostaglandin-H2 D-isomerase, transthyretin, vitamin D-binding protein, Zn-α-2-glycoprotein, or of an isoform thereof; and

(b) comparing the level of said at least one protein or protein isoform detected in step (a) with a range of levels of said at least one protein or protein isoform previously defined as characteristic for mammals suffering from AD, with a range of levels of said at least one protein or protein isoform previously defined as characteristic for mammals suffering from FTD, with a range of levels of said at least one protein or protein isoform previously defined as characteristic for mammals suffering from DLB, with a range of levels of said at least one protein or protein isoform previously defined as characteristic for mammals suffering from VAD, with a range of levels of said at least one protein or protein isoform previously defined as characteristic for mammals suffering from depression and with a range of levels of said at least one protein or protein isoform previously defined as characteristic for control mammals; and

(c) concluding from the comparison in step (b) whether the mammal is suffering from Alzheimer's disease, frontotemporal dementia, dementia with Lewy bodies, vascular dementia and/or depression, whereby a level of said at least one protein or protein isoform in a range previously defined as characteristic for mammals suffering from AD is an indication that said mammal is suffering from AD; and whereby a level of said at least one protein or protein isoform in a range previously defined as characteristic for mammals suffering from FTD is an indication that said mammal is suffering from FTD; and whereby a level of said at least one protein or protein isoform in a range previously defined as characteristic for mammals suffering from DLB is an indication that said mammal is suffering from DLB; and whereby a level of said at least one protein or protein isoform in a range previously defined as characteristic for mammals suffering from VAD is an indication that said mammal is suffering from VAD; and whereby a level of said at least one protein or protein isoform in a range previously defined as characteristic for mammals suffering from depression is an indication that said mammal is suffering from depression.

In particular, the present invention relates to a method for the screening, diagnosis and/or prognosis in a mammal of one or more neurological diseases, among which are Alzheimer's disease (AD), frontotemporal dementia (FTD), dementia with Lewy bodies (DLB), vascular dementia (VAD) and/or depression (D), to a method for identifying a mammal at risk of developing one or more neurological diseases, among which are Alzheimer's disease, frontotemporal dementia, dementia with Lewy bodies, vascular dementia and/or depression, or to a method for monitoring the effect of a therapy administered to a mammal having one or more neurological diseases, among which Alzheimer's disease, frontotemporal dementia, dementia with Lewy bodies, vascular dementia and/or depression. The method of the invention comprises the following steps:

(a) detecting, in the mammal under examination, the level of at least one of the following proteins: Apolipoprotein E (Apo E), α-1-antitrypsin, α-1-β glycoprotein, antithrombin III, Apolipoprotein A-I (Apo A-I), Apolipoprotein A-IV (Apo A-IV), Apolipoprotein J (Apo J), gelsolin, haptoglobin, hemopexin, Ig α-1 chain C region (heavy), kininogen, prostaglandin-H2 D-isomerase, transthyretin, vitamin D-binding protein, Zn-α-2-glycoprotein, or of an isoform thereof; and

(b) comparing the level of said at least one protein or protein isoform detected in step (a) with the level of said at least one protein or protein isoform in a control mammal; and

(c) concluding from the comparison in step (b) whether the mammal under examination is suffering from Alzheimer's disease, frontotemporal dementia, dementia with Lewy bodies, vascular dementia and/or depression, an altered level of said at least one protein or protein isoform being an indication of the mammal under examination suffering from Alzheimer's disease, dementia with Lewy bodies, frontotemporal dementia, vascular dementia and/or depression.

The present invention further relates to a method for the differential diagnosis in a mammal of different neurological diseases among which Alzheimer's disease, frontotemporal dementia, dementia with Lewy bodies, vascular dementia and/or depression. The method of the invention comprises the following steps:

(a) detecting, in the mammal under examination, the level of at least one of the following proteins: Apo E, α-1-antitrypsin, α-1-β glycoprotein, antithrombin III, Apo A-I, Apo A-IV, Apo J, gelsolin, haptoglobin, hemopexin, Ig α-1 chain C region (heavy), kininogen, prostaglandin-H2 D-isomerase, transthyretin, vitamin D-binding protein, Zn-α-2-glycoprotein, or of an isoform thereof; and

(b) comparing the level of said at least one protein or protein isoform detected in step (a) with the level of said at least one protein or protein isoform in a mammal suffering from another neurological disease; and

(c) concluding from the comparison in step (b) whether the mammal under examination is suffering from Alzheimer's disease, frontotemporal dementia, dementia with Lewy bodies, vascular dementia and/or depression.

The present invention is based on the finding that the levels of the above-indicated proteins are significantly altered in CSF samples obtained from AD patients, FTD patients, DLB patients, VAD patients and/or patients with depression compared to CSF samples obtained from control patients. The inventors further found that these protein profiles are differentially altered in CSF samples obtained from AD patients, FTD patients, DLB patients, VAD patients and/or patients with depression. The indication that the level of the above proteins differs between patients with AD, FTD, DLB, VAD, depression and/or control patients forms the basis for the development of diagnostic tests for the diagnosis and/or differential diagnosis of said neurological diseases in mammals.

More particularly, the present inventors were able to identify specific protein isoforms that are significantly altered in CSF samples obtained from AD patients, FTD patients, DLB patients, VAD patients and/or patients with depression compared to CSF samples from control patients. The inventors further found specific protein isoforms that are differentially altered in CSF samples obtained from AD patients, FTD patients, DLB patients, VAD patients and/or patients with depression.

A “protein isoform” refers to variants of a polypeptide that are encoded by the same gene, but that differ in their isoelectric point (pI) or molecular weight (MW), or both. Such isoforms can differ in their amino acid composition (e.g. as a result of alternative mRNA or premRNA processing, e.g. alternative splicing or limited proteolysis) and in addition, or alternatively, may arise from differential post-translational modification (e.g. glycosylation, acylation, phosphorylation) or can be metabolically altered (e.g. fragmented). In the present invention CSF from mammals with AD, FTD, DLB, VAD, or depression was analyzed for quantitative and qualitative detection of one or more protein isoform. A protein isoform of which the level is altered in CSF from mammals with AD, FTD, DLB, VAD, depression, or another neurological disease is also called a “neurological disease-associated protein isoform” or “NPI”. The NPIs of the present invention are listed in Tables 2, 3, 4, and 6.

An NPI, thus, is a protein comprising a peptide sequence described for that protein and which is further characterized as having a pI on 2-D gel electrophoresis of about the value stated in Table 2, 3, 4 or 6 for that NPI (preferably within about 10%, more preferably within about 5%, still more preferably within about 1% of the stated value) and having a MW on 2-D gel electrophoresis of about the value stated in Table 2, 3, 4 or 6 for that NPI (preferably within about 10%, more preferably within about 5%, still more preferably within about 1% of the stated value), if analyzed under similar circumstances.

Accordingly, the present invention provides a method for the screening, (differential) diagnosis and/or prognosis in a mammal of one or more neurological diseases, among which are Alzheimer's disease, frontotemporal dementia, dementia with Lewy bodies, vascular dementia and/or depression, a method for identifying a mammal at risk of developing Alzheimer's disease, frontotemporal dementia, dementia with Lewy bodies, vascular dementia and/or depression, or a method for monitoring the effect of a therapy administered to a mammal having Alzheimer's disease, frontotemporal dementia, dementia with Lewy bodies, vascular dementia and/or depression. The method of the invention comprises the following steps:

(a) detecting, in the mammal under examination, the level of at least one of the following protein isoforms (Table 2; Table 3; Table 4; Table 6):

Apo E: NPI 11, NPI 34, NPI 35, NPI 41, NPI 52, NPI 60, NPI 66, NPI 72, NPI 73, NPI 74, NPI 75, NPI 76m, NPI 77;

α-1-antitrypsin: NPI 1, NPI 42, NPI 43, NPI 44, NPI 59;

α-1-glycoprotein: NPI 2, NPI 3, NPI 31, NPI 48;

Antithrombin-III: NPI 4;

Apo A-I: NPI 5, NPI 6, NPI 7, NPI 37, NPI 69, NPI 70, NPI 71;

Apo A-IV: NPI 8, NPI 9, NPI 10;

Apo J: NPI 12, NPI 13, NPI 14, NPI 15, NPI 16;

Gelsolin: NPI 17;

Haptoglobin: NPI 18;

Hemopexin: NPI 19, NPI 20;

Ig α-1 chain C region (heavy): NPI 21, NPI 22;

Kininogen: NPI 23;

Prostaglandin-H2 D-isomerase: NPI 24, NPI 25;

Transthyretin: NPI 26, NPI 27, NPI 28m;

Vitamin D-binding protein: NPI 29, NPI 30;

Zn-α-2-glycoprotein: NPI 33;

NPI 32, NPI 36, NPI 38, NPI 39, NPI 40, NPI 45, NPI 46, NPI 47, NPI 49, NPI 50, NPI 51, NPI 53, NPI 54, NPI 55, NPI 56, NPI 57, NPI 58, NPI 61, NPI 62, NPI 63, NPI 64, NPI 65, NPI 67, NPI 68; and

(b) comparing the level of said at least one protein isoform detected in step (a) with a range of levels of said at least one protein isoform previously defined as characteristic for mammals suffering from AD, with a range of levels of levels of said at least one protein isoform previously defined as characteristic for mammals suffering from FTD, with a range of levels of levels of said at least one protein isoform previously defined as characteristic for mammals suffering from DLB, with a range of levels of levels of said at least one protein isoform previously defined as characteristic for mammals suffering from VAD, with a range of levels of levels of said at least one protein isoform previously defined as characteristic for mammals suffering from depression and with a range of levels of levels of said at least one protein isoform previously defined as characteristic for control mammals; and

(c) concluding from the comparison in step (b) whether the mammal is suffering from Alzheimer's disease, frontotemporal dementia, dementia with Lewy bodies, vascular dementia and/or depression, whereby a level of said at least one protein isoform in a range previously defined as characteristic for mammals suffering from AD is an indication that said mammal is suffering from AD; and whereby a level of said at least one protein isoform in a range previously defined as characteristic for mammals suffering from FTD is an indication that said mammal is suffering from FTD; and whereby a level of said at least one protein isoform in a range previously defined as characteristic for mammals suffering from DLB is an indication that said mammal is suffering from DLB; and whereby a level of said at least one protein isoform in a range previously defined as characteristic for mammals suffering from VAD is an indication that said mammal is suffering from VAD; and whereby a level of said at least one protein isoform in a range previously defined as characteristic for mammals suffering from depression is an indication that said mammal is suffering from depression.

The present invention thus provides a method for the screening, diagnosis and/or prognosis in a mammal of one or more neurological diseases, among which are Alzheimer's disease, frontotemporal dementia, dementia with Lewy bodies, vascular dementia and/or depression, a method for identifying a mammal at risk of developing Alzheimer's disease, frontotemporal dementia, dementia with Lewy bodies, vascular dementia and/or depression, or a method for monitoring the effect of a therapy administered to a mammal having Alzheimer's disease, frontotemporal dementia, dementia with Lewy bodies, vascular dementia and/or depression. The method of the invention comprises the following steps:

(b) detecting, in the mammal under examination, the level of at least one of the following protein isoforms (Table 2; Table 3; Table 4; Table 6):

Apo E: NPI 11, NPI 34, NPI 35, NPI 41, NPI 52, NPI 60, NPI 66, NPI 72, NPI 73, NPI 74, NPI 75, NPI 76m, NPI 77;

α-1-antitrypsin: NPI 1, NPI 42, NPI 43, NPI 44, NPI 59;

α-1-β glycoprotein: NPI 2, NPI 3, NPI 31, NPI 48;

Antithrombin-III: NPI 4;

Apo A-I: NPI 5, NPI 6, NPI 7, NPI 37, NPI 69, NPI 70, NPI 71;

Apo A-IV: NPI 8, NPI 9, NPI 10;

Apo J: NPI 12, NPI 13, NPI 14, NPI 15, NPI 16;

Gelsolin: NPI 17;

Haptoglobin: NPI 18;

Hemopexin: NPI 19, NPI 20;

Ig α-1 chain C region (heavy): NPI 21, NPI 22;

Kininogen: NPI 23;

Prostaglandin-H2 D-isomerase: NPI 24, NPI 25;

Transthyretin: NPI 26, NPI 27, NPI 28m;

Vitamin D-binding protein: NPI 29, NPI 30;

Zn-α-2-glycoprotein: NPI 33;

NPI 32, NPI 36, NPI 38, NPI 39, NPI 40, NPI 45, NPI 46, NPI 47, NPI 49, NPI 50, NPI 51, NPI 53, NPI 54, NPI 55, NPI 56, NPI 57, NPI 58, NPI 61, NPI 62, NPI 63, NPI 64, NPI 65, NPI 67, NPI 68; and

(c) comparing the level of said at least one protein isoform detected in step (a) with the level of said at least one protein isoform in a control mammal; and

(d) concluding from the comparison in step (b) whether the mammal under examination is suffering from Alzheimer's disease, dementia with Lewy bodies, frontotemporal dementia, vascular dementia and/or depression, an altered level of said at least one protein isoform being an indication of the mammal under examination suffering from Alzheimer's disease, dementia with Lewy bodies, frontotemporal dementia, vascular dementia and/or depression.

The present invention further provides a method for the differential diagnosis in a mammal of one or more neurological diseases, among which are Alzheimer's disease, frontotemporal dementia, dementia with Lewy bodies, vascular dementia and/or of depression. The method of the invention comprises the following steps:

(a) detecting, in the mammal under examination, the level of at least one of the following protein isoforms (Table 2; Table 3; Table 4; Table 6):

Apo E: NPI 11, NPI 34, NPI 35, NPI 41, NPI 52, NPI 60, NPI 66, NPI 72, NPI 73, NPI 74, NPI 75, NPI 76m, NPI 77;

α-1-antitrypsin: NPI 1, NPI 42, NPI 43, NPI 44, NPI 59;

α-1-β glycoprotein: NPI 2, NPI 3, NPI 31, NPI 48;

Antithrombin-III: NPI 4;

Apo A-I: NPI 5, NPI 6, NPI 7, NPI 37, NPI 69, NPI 70, NPI 71;

Apo A-IV: NPI 8, NPI 9, NPI 10;

Apo J: NPI 12, NPI 13, NPI 14, NPI 15, NPI 16;

Gelsolin: NPI 17;

Haptoglobin: NPI 18;

Hemopexin: NPI 19, NPI 20;

Ig α-1 chain C region (heavy): NPI 21, NPI 22;

Kininogen: NPI 23;

Prostaglandin-H2 D-isomerase: NPI 24, NPI 25;

Transthyretin: NPI 26, NPI 27, NPI 28m;

Vitamin D-binding protein: NPI 29, NPI 30;

Zn-α-2-glycoprotein: NPI 33;

NPI 32, NPI 36, NPI 38, NPI 39, NPI 40, NPI 45, NPI 46, NPI 47, NPI 49, NPI 50, NPI 51, NPI 53, NPI 54, NPI 55, NPI 56, NPI 57, NPI 58, NPI 61, NPI 62, NPI 63, NPI 64, NPI 65, NPI 67, NPI 68; and

(b) comparing the level of said at least one protein isoform detected in step (a) with the level of said at least one protein isoform in a mammal suffering from another neurological disease; and

(c) concluding from the comparison in step (b) whether the mammal under examination is suffering from Alzheimer's disease, frontotemporal dementia, dementia with Lewy bodies, vascular dementia and/or depression.

The proteins and protein isoforms as indicated above thus present new biomarkers for use in the diagnosis of neurological diseases.

The mammal examined in the present invention may be a non-human mammal, such as (but not limited to) a cow, a pig, a sheep, a goat, a horse, a monkey, a rabbit, a hare, a dog, a cat, a mouse, a rat, an elk, a deer, or a tiger. In a preferred embodiment, the mammal is a primate. In another preferred embodiment the mammal is a human, more preferably the mammal is a human adult.

The method of the present invention can also be used in animal models representative for a human disease, for example, for use in drug screening. The animal model on which the method of the present invention can be applied can be any model of an animal in which the body control system is directed by CNS. The animal thus may belong to the Platyhelminthes, Aschelminthes, Annelida, Arthropoda, Mollusca, Echinodermata, Acrania, Cyclostomata, Chondrichthyes, Osteichthyes, Amphibia, Reptilia, Aves and Mammalia. In a preferred embodiment, the animal in the animal model is a mouse, a rat, a monkey, a rabbit, a worm, or a fly.

A “control mammal”, as defined in the present invention is a mammal of the same species as the mammal under examination which is free from AD, FTD, DLB, VAD and depression. Preferably, the control mammal is free from any neurological disease. A mammalian species as used in the present invention refers to the lowest taxonomic classification used that differentiates between mammals that can actively reproduce with one another and produce fertile offspring. “A mammal of the same species” as used in the present invention, therefore, is a mammal that can actively reproduce with the mammal suspected to suffer from a neurological disease. In a preferred embodiment, a reference level range for the control mammal can be determined for a certain NPI in a mammal free from AD, FTD, DLB, VAD and depression. The level obtained in the mammal suspected to suffer from AD, FTD, DLB, VAD and/or depression can then be compared with the previously determined reference level range. The term “level” or “levels”, as used in the present invention, refers to the presence or absence and/or the amount of a protein or protein isoform. A change in the level of a protein or protein isoform refers to a measurable increase or decrease, including total absence or presence, in the protein or protein isoform level when compared to control mammals or to mammals suffering from another neurological disease. For any given NPI, the level obtained upon analyzing a mammal suspected of suffering a certain neurological disease relative to the level obtained upon analyzing a control mammal or a mammal suffering from another neurological disease will depend on the particular analytical protocol and detection technique that is used. Accordingly, those skilled in the art will understand that, based on the present description, any laboratory can establish, for a given NPI, a suitable “reference range”, “reference level range”, “level range” or “range of levels” (those terms are used interchangeable) characteristic for control mammals or mammals suffering from AD, FTD, DLB, VAD and/or depression according to the analytical protocol and detection technique in use. The level obtained for the mammal under diagnosis can then be compared with this reference range and, based on this comparison, a conclusion can be drawn as to which neurological disease the mammal is suffering from. Those skilled in the art will also know how to establish, for a given NPI, a cut-off value suitable for differentiating mammals suffering from AD, DLB, FTD, VAD and/or depression from control mammals, or suitable for differentiating mammals suffering from AD, DLB, FTD, VAD and/or depression from each other. Methods for defining cut-off values include (but are not limited to) the methods described by IFCC (1987). [nog aanvullen Geert De Meyer]

An “altered level of the protein or protein isoform” as used in the present invention refers to the appearance or disappearance of the protein or protein isoform under examination (in the present invention also referred to as qualitative difference or QL; Tables 2, 3, 4 and 6) or to the increase or the decrease of the protein or protein isoform under examination (in the present invention also referred to as quantitative difference or QN; Tables 2 and 6) in mammals with a certain neurological disease relative to control mammals or relative to mammals suffering from another neurological disease. In the method of the present invention, at least one of the proteins or protein isoforms associated with one or more neurological diseases, among which are Alzheimer's disease, frontotemporal dementia, dementia with Lewy bodies, vascular dementia and/or depression (indicated in Tables 2, 3, 4, and/or 6) is detected. It is clear that also more than one of the above proteins or protein isoforms can be detected simultaneously. Detection of an appropriate combination of more than one biological marker will often increase the specificity and sensitivity of the method. Therefore, in a preferred embodiment, a combination of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70 or at least 80 protein isoforms is detected in the method of the invention. When multiple proteins or protein isoforms are detected this is also called a protein profile. The term “protein profile” refers to a group of specific proteins present in samples obtained from mammals with neurological diseases in which differences can be detected when compared to control mammals. A disease-specific protein profile is obtained by comparing the level of a variety of proteins in a sample taken from a mammal suffering from a certain neurological disease to the levels found in samples taken from a control mammal or mammals suffering from another neurological disease. The proteins that comprise the profile may be unaltered, increased, decreased, present or absent with respect to the control mammal or the mammal suffering from another neurological disease. In any of the above methods, detection of at least one NPI may optionally be combined with detection of one or more additional known biomarkers for neurological diseases, including but not limited to amyloid β peptides, tau, phospho-tau, synuclein, Rab3a, and neural thread protein.

“Diagnosis” as used in the present invention refers to diagnosis, prognosis, monitoring, selecting participants in clinical trials, and identifying patients most likely to respond to a particular therapeutic treatment. Treatment refers to therapy, prevention, and prophylaxis. The method of the invention can also be used for monitoring the effect of therapy administered to a mammal, also called therapeutic monitoring, and patient management. Changes in the level of the protein and/or protein isoform as identified above and associated with one or more neurological diseases, among which are Alzheimer's disease, frontotemporal dementia, dementia with Lewy bodies, vascular dementia and/or depression, can also be used to evaluate the response of a mammal to drug treatment. In this way, new treatment regimes can also be developed by examining the level of the protein or protein isoform in a mammal. The method of the present invention can thus assist in monitoring a clinical study, for example, for evaluation of a certain therapy for AD, FTD, DLB, VAD, and/or depression. In this case, a chemical compound is tested for its ability to normalize the level of a NPI in a mammal having AD, FTD, DLB, VAD, and/or depression to levels found in control mammals. In a treated mammal, a chemical compound can be tested for its ability to maintain the NPI level at or near the level seen in control mammals.

The present invention further provides for methods for the differential diagnosis of neurological diseases. The term “differential diagnosis” means that individuals suffering from a certain neurological disease are discriminated from individuals suffering from another neurological disease. The method of the present invention allows the differential diagnosis of an individual suffering from Alzheimer's disease, from frontotemporal dementia, from dementia with Lewy bodies, from vascular dementia and/or from depression. In a specific embodiment, the present invention allows the differential diagnosis of an individual suffering from Alzheimer's disease (AD) versus an individual suffering from frontotemporal dementia (FTD). In another specific embodiment, the present invention allows the differential diagnosis of an individual suffering from Alzheimer's disease (AD) versus an individual suffering from dementia with Lewy bodies (DLB). In another specific embodiment, the present invention allows the differential diagnosis of an individual suffering from Alzheimer's disease (AD) versus an individual suffering from vascular dementia (VAD). In another specific embodiment, the present invention allows the differential diagnosis of an individual suffering from Alzheimer's disease (AD) versus an individual suffering from depression. In another specific embodiment, the present invention allows the differential diagnosis of an individual suffering from frontotemporal dementia (FTD) versus an individual suffering from dementia with Lewy bodies (DLB). In another specific embodiment, the present invention allows the differential diagnosis of an individual suffering from frontotemporal dementia (FTD) versus an individual suffering from vascular dementia (VAD). In another specific embodiment, the present invention allows the differential diagnosis of an individual suffering from frontotemporal dementia (FTD) versus an individual suffering from depression. In another specific embodiment, the present invention allows the differential diagnosis of an individual suffering from dementia with Lewy bodies (DLB) versus an individual suffering from vascular dementia (VAD). In another specific embodiment, the present invention allows the differential diagnosis of an individual suffering from dementia with Lewy bodies (DLB) versus an individual suffering from depression. In another specific embodiment, the present invention allows the differential diagnosis of an individual suffering from vascular dementia (VAD) versus an individual suffering from depression.

Alzheimer's disease, frontotemporal dementia, dementia with Lewy bodies, vascular dementia and depression as well as other neurological diseases have been described in detail by Wilson et al. (1991) and McKeith et al. (1999).

Different groups of NPIs, each with a different behaviour (appearance, disappearance, increase or decrease) in the various neurological diseases, were isolated and identified (see Tables 2, 3, 4 and 6 and the example section).

A first group comprises the NPIs that are decreased in mammals having AD as compared to control mammals (C>AD). This group includes NPI 1, NPI 16 and NPI 25 (Table 2). Accordingly, in one embodiment, the present invention relates to a method for the screening, diagnosis or prognosis in a mammal of Alzheimer's disease, for identifying a mammal at risk of developing Alzheimer's disease, or for monitoring the effect of therapy administered to a mammal having Alzheimer's disease, said method comprising the following steps:

(a) detecting, in said mammal, the level of at least one of the following protein isoforms: NPI 1, NPI 16, NPI 25; and

(b) comparing the level of said at least one protein isoform detected in step (a) with the level of said at least one protein isoform in a control mammal; and

(c) concluding from the comparison in step (b) whether the mammal under examination is suffering from Alzheimer's disease, a decreased level of said at least one protein isoform compared to the level of said at least one protein isoform in a control mammal being an indication of the mammal under examination suffering from Alzheimer's disease.

A second group comprises the NPIs that are decreased in mammals having FTD as compared to control mammals (C>FTD). This group includes NPI 5, NPI 6, NPI 12, NPI 17 and NPI 24 (Table 2). Accordingly, in one embodiment, the present invention relates to a method for the screening, diagnosis or prognosis in a mammal of frontotemporal dementia, for identifying a mammal at risk of developing frontotemporal dementia, or for monitoring the effect of therapy administered to a mammal having frontotemporal dementia, said method comprising the following steps:

(a) detecting, in said mammal, the level of at least one of the following protein isoforms: NPI 5, NPI 6, NPI 12, NPI 17, NPI 24; and

(b) comparing the level of said at least one protein isoform detected in step (a) with the level of said at least one protein isoform in a control mammal; and

(c) concluding from the comparison in step (b) whether the mammal under examination is suffering from frontotemporal dementia, a decreased level of said at least one protein isoform compared to the level of said at least one protein isoform in a control mammal being an indication of the mammal under examination suffering from frontotemporal dementia.

A third group comprises the NPIs that are increased in mammals having FTD as compared to control mammals (FTD>C). This group includes NPI 4, NPI 8, NPI 9, NPI 10, NPI 18, NPI 19, NPI 20, NPI 22, NPI 23, NPI 28m and NPI 70 (Table 2).

Accordingly, in one embodiment, the present invention relates to a method for the screening, diagnosis or prognosis in a mammal of frontotemporal dementia, for identifying a mammal at risk of developing frontotemporal dementia, or for monitoring the effect of therapy administered to a mammal having frontotemporal dementia, said method comprising the following steps:

(a) detecting, in said mammal, the level of at least one of the following protein isoforms: NPI 4, NPI 8, NPI 9, NPI 10, NPI 18, NPI 19, NPI 20, NPI 22, NPI 23, NPI 28m, NPI 70; and

(b) comparing the level of said at least one protein isoform detected in step (a) with the level of said at least one protein isoform in a control mammal; and

(c) concluding from the comparison in step (b) whether the mammal under examination is suffering from frontotemporal dementia, an increased level of said at least one protein isoform compared to the level of said at least one protein isoform in a control mammal being an indication of the mammal under examination suffering from frontotemporal dementia.

A fourth group comprises the NPIs that are increased in mammals having AD as compared to mammals having FTD (AD>FTD). This group includes NPI 5, NPI 6 and NPI 26 (Table 2). Accordingly, in one embodiment, the present invention relates to a method for the differential diagnosis in a mammal of Alzheimer's disease versus frontotemporal dementia, said method comprising the following steps:

(a) detecting, in said mammal, the level of at least one of the following protein isoforms: NPI 5, NPI 6, NPI 26;

(b) comparing the level of said at least one protein isoform detected in step (a) with a previously defined cut-off value suitable for differentiating mammals suffering from AD versus mammals suffering from FTD; and

(c) concluding from the comparison in step (b) whether the mammal is suffering from AD or from FTD, whereby a level of said at least one protein isoform above the cut-off value being an indication of the mammal suffering from Alzheimer's disease; and whereby a level of said at least one protein isoform below the cut-off value being an indication of the mammal suffering from FTD.

A fifth group comprises the NPIs that are decreased in mammals having AD as compared to mammals having FTD (FTD>AD). This group includes NPI 2, NPI 3, NPI 7, NPI 8, NPI 9, NPI 11, NPI 13, NPI 14, NPI 15, NPI 16, NPI 21, NPI 22, NPI 25, NPI 27, NPI 28m, NPI 29, NPI 30, NPI 69 and NPI 71 (Table 2). Accordingly, in one embodiment, the present invention relates to a method for the differential diagnosis in a mammal of Alzheimer's disease versus frontotemporal dementia, said method comprising the following steps:

(a) detecting, in said mammal, the level of at least one of the following protein isoforms: NPI 2, NPI 3, NPI 7, NPI 8, NPI 9, NPI 11, NPI 13, NPI 14, NPI 15, NPI 16, NPI 21, NPI 22, NPI 25, NPI 27, NPI 28m, NPI 29, NPI 30, NPI 69, NPI 71;

(b) comparing the level of said at least one protein isoform detected in step (a) with a previously defined cut-off value suitable for differentiating mammals suffering from AD versus mammals suffering from FTD; and

(c) concluding from the comparison in step (b) whether the mammal is suffering from AD or from FTD, whereby a level of said at least one protein isoform below the cut-off value being an indication of the mammal suffering from Alzheimer's disease; and whereby a level of said at least one protein isoform above the cut-off value being an indication of said mammal suffering from FTD.

A sixth group comprises the NPIs that are decreased in mammals having AD as compared to mammals having depression (AD<D). This group includes NPI 6, NPI 12, NPI 23, NPI 31, NPI 32, NPI 33, NPI 34, NPI 35, NPI 36, NPI 37, NPI 38, NPI 40, NPI 41, NPI 42, NPI 43, NPI 44, NPI 45, NPI 46, NPI 47, NPI 48, NPI 51, NPI 52, NPI 53, NPI 54, NPI 55, NPI 56, NPI 58, NPI 59, NPI 60, NPI 61, NPI 63, NPI 68 and NPI 69 (Table 2; Table 6). Accordingly, in one embodiment, the present invention relates to a method for the differential diagnosis in a mammal of Alzheimer's disease versus depression, said method comprising the following steps:

(a) detecting, in said mammal, the level of at least one of the following protein isoforms: NPI 6, NPI 12, NPI 23, NPI 31, NPI 32, NPI 33, NPI 34, NPI 35, NPI 36, NPI 37, NPI 38, NPI 40, NPI 41, NPI 42, NPI 43, NPI 44, NPI 45, NPI 46, NPI 47, NPI 48, NPI 51, NPI 52, NPI 53, NPI 54, NPI 55, NPI 56, NPI 58, NPI 59, NPI 60, NPI 61, NPI 63, NPI 68, NPI 69;

(b) comparing the level of said at least one protein isoform detected in step (a) with a previously defined cut-off value suitable for differentiating mammals suffering from AD versus mammals suffering from depression; and

(c) concluding from the comparison in step (b) whether the mammal is suffering from Alzheimer's disease or from depression, whereby a level of said at least one protein isoform below the cut-off value being an indication of the mammal suffering from Alzheimer's disease; and whereby a level of said at least one protein isoform above the cut-off value being an indication of said mammal suffering from depression.

An seventh group comprises the NPIs that are increased in mammals having AD as compared to mammals having depression (AD>D). This group includes NPI 39, NPI 49, NPI 50, NPI 57, NPI 62, NPI 64, NPI 65, NPI 66 and NPI 67 (Table 2; Table 6). Accordingly, in one embodiment, the present invention relates to a method for the differential diagnosis in a mammal of Alzheimer's disease versus depression, said method comprising the following steps:

(a) detecting, in said mammal, the level of at least one of the following protein isoforms: NPI 39, NPI 49, NPI 50, NPI 57, NPI 62, NPI 64, NPI 65, NPI 66, NPI 67;

(b) comparing the level of said at least one protein isoform detected in step (a) with a previously defined cut-off value suitable for differentiating mammals suffering from AD versus mammals suffering from depression; and

(c) concluding from the comparison in step (b) whether the mammal is suffering from Alzheimer's disease or from depression, whereby a level of said at least one protein isoform above the cut-off value being an indication of the mammal suffering from Alzheimer's disease; and whereby a level of said at least one protein isoform below the cut-off value being an indication of said mammal suffering from depression.

A eighth group comprises the NPIs that are decreased in mammals having AD as compared to mammals having VAD (VAD>AD). This group includes NPI 7, NPI 74, and NPI 76m (Table 2). Accordingly, in one embodiment, the present invention relates to a method for the differential diagnosis in a mammal of Alzheimer's disease versus Vascular dementia, said method comprising the following steps:

(a) detecting, in said mammal, the level of at least one of the following protein isoforms: NPI 7, NPI 74, NPI 76m;

(b) comparing the level of said at least one protein isoform detected in step (a) with a previously defined cut-off value suitable for the differentiating mammals suffering from AD versus mammals suffering from VAD; and

(c) concluding from the comparison in step (b) whether the mammal is suffering from Alzheimer's disease or from VAD, whereby a level of said at least one protein isoform below the cut-off value being an indication of the mammal suffering from Alzheimer's disease; and whereby a level of said at least one protein isoform above the cut-off value being an indication of said mammal suffering from VAD.

A ninth group comprises the NPIs that are increased in mammals having AD as compared to mammals having VAD (AD>VAD). This group includes NPI 5 (Table 2). Accordingly, in one embodiment, the present invention relates to a method for the differential diagnosis in a mammal of Alzheimer's disease versus vascular dementia, said method comprising the following steps:

(a) detecting, in said mammal, the level of the following protein isoform: NPI 5;

(b) comparing the level of said protein isoform detected in step (a) with a previously defined cut-off value suitable for differentiating mammals suffering from AD versus mammals suffering from VAD; and

(c) concluding from the comparison in step (b) whether the mammal is suffering from Alzheimer's disease or from VAD, whereby a level of said protein isoform above the cut-off value being an indication of the mammal suffering from Alzheimer's disease; and whereby a level of said protein isoform below the cut-off value being an indication of said mammal suffering from VAD.

The level of one or more NPIs can be determined in vitro as well as in vivo. The method for the in vitro detection of the level of the NPI in a mammal comprises the steps of obtaining a sample from said mammal, determining the level of the NPI in said sample and comparing the obtained level in said sample with a range of levels of said NPI characteristic for samples taken from control mammals or from mammals suffering from another neurological disease.

The term “sample” refers to any source of biological material, for instance body fluids, brain extract, peripheral blood or any other sample comprising the NPI. In a preferred embodiment, the level of the NPI is determined in vitro by analysis of the level of the NPI in a body fluid sample of the mammal. The term “body fluid” refers to all fluids that are present in the mammalian body including, but not limited to, blood, lymph, urine, and cerebrospinal fluid (CSF) comprising the NPI. The blood sample may include a plasma sample or a serum sample.

In a preferred embodiment of the present invention the level of the NPI is determined in a cerebrospinal fluid sample taken from the mammal. The term “cerebrospinal fluid” or “CSF” is intended to include whole cerebrospinal fluid or derivatives of fractions thereof well known to those skilled in the art. Thus, a cerebrospinal fluid sample can include various fractionated forms of cerebrospinal fluid or can include various diluents or detergents added to facilitate storage or processing in a particular assay. Such diluents and detergents are well known to those skilled in the art and include various buffers, preservatives and the like.

Accordingly, the present invention relates to a method as described above, comprising the steps of:

(a) obtaining a cerebrospinal fluid sample from the mammal under examination; and

(b) detecting, in said cerebrospinal fluid sample, at least one of the following protein isoforms (Table 2; Table 3; Table 4; Table 6):

Apo E: NPI 11, NPI 34, NPI 35, NPI 41, NPI 52, NPI 60, NPI 66, NPI 72, NPI 73, NPI 74, NPI 75, NPI 76m, NPI 77;

α-1-antitrypsin: NPI 1, NPI 42, NPI 43, NPI 44, NPI 59;

α-1-β glycoprotein: NPI 2, NPI 3, NPI 31, NPI 48;

Antithrombin-III: NPI 4;

Apo A-I: NPI 5, NPI 6, NPI 7, NPI 37, NPI 69, NPI 70, NPI 71;

Apo A-IV: NPI 8, NPI 9, NPI 10;

Apo J: NPI 12, NPI 13, NPI 14, NPI 15, NPI 16;

Gelsolin: NPI 17;

Haptoglobin: NPI 18;

Hemopexin: NPI 19, NPI 20;

Ig α-1 chain C region (heavy): NPI 21, NPI 22;

Kininogen: NPI 23;

Prostaglandin-H2 D-isomerase: NPI 24, NPI 25;

Transthyretin: NPI 26, NPI 27, NPI 28m;

Vitamin D-binding protein: NPI 29, NPI 30;

Zn-α-2-glycoprotein: NPI 33;

NPI 32, NPI 36, NPI 38, NPI 39, NPI 40, NPI 45, NPI 46, NPI 47, NPI 49, NPI 50, NPI 51, NPI 53, NPI 54, NPI 55, NPI 56, NPI 57, NPI 58, NPI 61, NPI 62, NPI 63, NPI 64, NPI 65, NPI 67, NPI 68; and

(c) comparing the level of said at least one protein isoform detected in step (b) with a range of levels characteristic for CSF samples from control mammals or from mammals suffering from another neurological disease; and

(d) concluding from the comparison in step (c) whether the mammal under examination is suffering from Alzheimer's disease, frontotemporal dementia, dementia with Lewy bodies, vascular dementia and/or depression, whereby a level of said at least one protein isoform in a range previously defined as characteristic for mammals suffering from AD is an indication that said mammal is suffering from AD; and whereby a level of said at least one protein isoform in a range previously defined as characteristic for mammals suffering from FTD is an indication that said mammal is suffering from FTD; and whereby a level of said at least one protein isoform in a range previously defined as characteristic for mammals suffering from DLB is an indication that said mammal is suffering from DLB; and whereby a level of said at least one protein isoform in a range previously defined as characteristic for mammals suffering from VAD is an indication that said mammal is suffering from VAD; and whereby a level of said at least one protein isoform in a range previously defined as characteristic for mammals suffering from depression is an indication that said mammal is suffering from depression.

Accordingly, in a specific embodiment, the present invention relates to a method for the screening, diagnosis or prognosis in a mammal of Alzheimer's disease, for identifying a mammal at risk of developing Alzheimer's disease, or for monitoring the effect of therapy administered to a mammal having Alzheimer's disease, said method comprising the following steps:

(a) detecting, in a CSF sample taken from said mammal, the level of at least one of the following protein isoforms (Table 2): NPI 1, NPI 16, NPI 25; and

(b) comparing the level of said at least one protein isoform detected in step (a) with the level of said at least one protein isoform in a CSF sample taken from a control mammal; and

(c) concluding from the comparison in step (b) whether the mammal under examination is suffering from Alzheimer's disease, a decreased level of said at least one protein isoform compared to the level of said at least one protein isoform in a CSF sample taken from a control mammal being an indication of the mammal under examination suffering from Alzheimer's disease.

In another embodiment, the present invention relates to a method for the screening, diagnosis or prognosis in a mammal of frontotemporal dementia, for identifying a mammal at risk of developing frontotemporal dementia, or for monitoring the effect of therapy administered to a mammal having frontotemporal dementia, said method comprising the following steps:

(a) detecting, in a CSF sample taken from said mammal, the level of at least one of the following protein isoforms (Table 2): NPI 5, NPI 6, NPI 12, NPI 17, NPI 24; and

(b) comparing the level of said at least one protein isoform detected in step (a) with the level of said at least one protein isoform in a CSF sample taken from a control mammal; and

(c) concluding from the comparison in step (b) whether the mammal under examination is suffering from frontotemporal dementia, a decreased level of said at least one protein isoform compared to the level of said at least one protein isoform in a CSF sample taken from a control mammal being an indication of the mammal under examination suffering from frontotemporal dementia.

In another embodiment, the present invention relates to a method for the screening, diagnosis or prognosis in a mammal of frontotemporal dementia, for identifying a mammal at risk of developing frontotemporal dementia, or for monitoring the effect of therapy administered to a mammal having frontotemporal dementia, said method comprising the following steps:

(a) detecting, in a CSF sample taken from said mammal, the level of at least one of the following protein isoforms (Table 2): NPI 4, NPI 8, NPI 9, NPI 10, NPI 18, NPI 19, NPI 20, NPI 22, NPI 23, NPI 28m, NPI 70; and

(b) comparing the level of said at least one protein isoform detected in step (a) with the level of said at least one protein isoform in a CSF sample taken from a control mammal; and

(c) concluding from the comparison in step (b) whether the mammal under examination is suffering from frontotemporal dementia, an increased level of said at least one protein isoform compared to the level of said at least one protein isoform in a CSF sample taken from a control mammal being an indication of the mammal under examination suffering from frontotemporal dementia.

In another embodiment, the present invention relates to a method for the differential diagnosis in a mammal of Alzheimer's disease versus frontotemporal dementia, said method comprising the following steps:

(a) detecting, in a CSF sample taken from said mammal, the level of at least one of the following protein isoforms (Table 2): NPI 5, NPI 6, NPI 26;

(b) comparing the level of said at least one protein isoform detected in step (a) with a previously defined cut-off value suitable for differentiating mammals suffering from AD versus mammals suffering from FTD; and

(a) concluding from the comparison in step (b) whether the mammal is suffering from AD or from FTD, whereby a level of said at least one protein isoform above the cut-off value being an indication of the mammal suffering from Alzheimer's disease; and whereby a level of said at least one protein isoform below the cut-off value being an indication of the mammal suffering from FTD.

In another embodiment, the present invention relates to a method for the differential diagnosis in a mammal of Alzheimer's disease versus frontotemporal dementia, said method comprising the following steps:

(a) detecting, in a CSF sample taken from said mammal, the level of at least one of the following protein isoforms (Table 2): NPI 2, NPI 3, NPI 7, NPI 8, NPI 9, NPI 11, NPI 13, NPI 14, NPI 15, NPI 16, NPI 21, NPI 22, NPI 25, NPI 27, NPI 28m, NPI 29, NPI 30, NPI 69, NPI 71;

(b) comparing the level of said at least one protein isoform detected in step (a) with a previously defined cut-off value suitable for differentiating mammals suffering from AD versus mammals suffering from FTD; and

(c) concluding from the comparison in step (b) whether the mammal is suffering from AD or from FTD, whereby a level of said at least one protein isoform below the cut-off value being an indication of the mammal suffering from Alzheimer's disease; and whereby a level of said at least one protein isoform above the cut-off value being an indication of said mammal suffering from FTD.

In another embodiment, the present invention relates to a method for the differential diagnosis in a mammal of Alzheimer's disease versus depression, said method comprising the following steps:

(a) detecting, in a CSF sample taken from said mammal, the level of at least one of the following protein isoforms (Table 2; Table 6): NPI 6, NPI 12, NPI 23, NPI 31, NPI 32, NPI 33, NPI 34, NPI 35, NPI 36, NPI 37, NPI 38, NPI 40, NPI 41, NPI 42, NPI 43, NPI 44, NPI 45, NPI 46, NPI 47, NPI 48, NPI 51, NPI 52, NPI 53, NPI 54, NPI 55, NPI 56, NPI 58, NPI 59, NPI 60, NPI 61, NPI 63, NPI 68,

NPI 69;

(b) comparing the level of said at least one protein isoform detected in step (a) with a previously defined cut-off value suitable for differentiating mammals suffering from AD versus mammals suffering from depression; and

(c) concluding from the comparison in step (b) whether the mammal is suffering from Alzheimer's disease or from depression, whereby a level of said at least one protein isoform below the cut-off value being an indication of the mammal suffering from Alzheimer's disease; and whereby a level of said at least one protein isoform above the cut-off value being an indication of said mammal suffering from depression.

In another embodiment, the present invention relates to a method for the differential diagnosis in a mammal of Alzheimer's disease versus depression, said method comprising the following steps:

(a) detecting, in a CSF sample taken from said mammal, the level of at least one of the following protein isoforms (Table 2; Table 6): NPI 39, NPI 49, NPI 50, NPI 57, NPI 62, NPI 64, NPI 65, NPI 66, NPI 67;

(b) comparing the level of said at least one protein isoform detected in step (a) with a previously defined cut-off value suitable for differentiating mammals suffering from AD versus mammals suffering from depression; and

(c) concluding from the comparison in step (b) whether the mammal is suffering from Alzheimer's disease or from depression, whereby a level of said at least one protein isoform above the cut-off value being an indication of the mammal suffering from Alzheimer's disease; and whereby a level of said at least one protein isoform below the cut-off value being an indication of said mammal suffering from depression.

In another embodiment, the present invention relates to a method for the differential diagnosis in a mammal of Alzheimer's disease versus vascular dementia, said method comprising the following steps:

(a) detecting, in a CSF sample taken from said mammal, the level of at least one of the following protein isoforms (Table 2): NPI 7, NPI 74, NPI 76m;

(b) comparing the level of said at least one protein isoform detected in step (a) with a previously defined cut-off value suitable for the differentiating mammals suffering from AD versus mammals suffering from VAD; and

(c) concluding from the comparison in step (b) whether the mammal is suffering from Alzheimer's disease or from VAD, whereby a level of said at least one protein isoform below the cut-off value being an indication of the mammal suffering from Alzheimer's disease; and whereby a level of said at least one protein isoform above the cut-off value being an indication of said mammal suffering from VAD.

In another embodiment, the present invention relates to a method for the differential diagnosis in a mammal of Alzheimer's disease versus vascular dementia, said method comprising the following steps:

(a) detecting, in a CSF sample taken from said mammal, the level of the following protein isoform (Table 2): NPI 5;

(b) comparing the level of said protein isoform detected in step (a) with a previously defined cut-off value suitable for differentiating mammals suffering from AD versus mammals suffering from VAD; and

(c) concluding from the comparison in step (b) whether the mammal is suffering from Alzheimer's disease or from VAD, whereby a level of said protein isoform above the cut-off value being an indication of the mammal suffering from Alzheimer's disease; and whereby a level of said protein isoform below the cut-off value being an indication of said mammal suffering from VAD.

The present invention additionally provides a composition comprising at least one of the following isolated protein isoforms associated with one or more neurological diseases, among which are Alzheimer's disease, frontotemporal dementia, dementia with Lewy bodies, vascular dementia and/or depression, or a fragment thereof:

Apo E: NPI 11, NPI 34, NPI 35, NPI 41, NPI 52, NPI 60, NPI 66, NPI 72, NPI 73, NPI 74, NPI 75, NPI 76m, NPI 77;

α-1-antitrypsin: NPI 1, NPI 42, NPI 43, NPI 44, NPI 59;

α-1-β glycoprotein: NPI 2, NPI 3, NPI 31, NPI 48;

Antithrombin-III: NPI 4;

Apo A-I: NPI 5, NPI 6, NPI 7, NPI 37, NPI 69, NPI 70, NPI 71;

Apo A-IV: NPI 8, NPI 9, NPI 10;

Apo J: NPI 12, NPI 13, NPI 14, NPI 15, NPI 16;

Gelsolin: NPI 17;

Haptoglobin: NPI 18;

Hemopexin: NPI 19, NPI 20;

Ig α-1 chain C region (heavy): NPI 21, NPI 22;

Kininogen: NPI 23;

Prostaglandin-H2 D-isomerase: NPI 24, NPI 25;

Transthyretin: NPI 26, NPI 27, NPI 28m;

Vitamin D-binding protein: NPI 29, NPI 30;

Zn-α-2-glycoprotein: NPI 33;

NPI 32, NPI 36, NPI 38, NPI 39, NPI 40, NPI 45, NPI 46, NPI 47, NPI 49, NPI 50, NPI 51, NPI 53, NPI 54, NPI 55, NPI 56, NPI 57, NPI 58, NPI 61, NPI 62, NPI 63, NPI 64, NPI 65, NPI 67, NPI 68.

An NPI is isolated when it is present in a preparation that is substantially free of other proteins, i.e., a preparation in which less than 30% (particularly less than 20%, more particularly less than 10%, more particularly less than 5%, more particularly less than 1%) of the total protein present is contaminating protein(s). The NPI identified herein can be isolated and purified by standard methods including chromatography (e.g. ion exchange, affinity, and sizing column chromatography), centrifugation, differential solubility, or by any other technique for the purification of proteins. Alternatively, once a recombinant nucleic acid that encodes the NPI is identified, the entire amino acid sequence of the NPI can be deduced from the nucleotide sequence of the gene-coding region contained in the recombinant nucleic acid. As a result, the protein can be synthesized by standard chemical methods or by any recombinant technique known in the art.

The proteins or protein isoforms that are associated with one or more neurological diseases among which Alzheimer's disease, frontotemporal dementia, dementia with Lewy bodies, vascular dementia and/or depression may be detected by any method known to those skilled in the art. They can be identified by their structure, by partial amino acid sequence determination, by functional assay, by enzyme assay, by various immunological methods, or by biochemical methods such as capillary electrophoresis, high performance liquid chromatography (HPLC), thin layer chromatography (TLC), hyperdiffusion chromatography, two-dimensional liquid phase electrophoresis (2-D-LPE; Davidsson et al. 1999) or by their migration pattern in gel electrophoreses. Sodium dodecyl sulfate-polyacrylamide gel electrophoresis (SDS-PAGE) is a widely used approach for separating proteins from complex mixtures (Patterson and Aebersold, 1995). It can be performed in one- or two-dimensional (2-D) configuration. For less complicated protein preparation, one-dimensional SDS-PAGE is preferred over 2-D gels, because it is simpler. However, SDS-PAGE often results in migrating or overlapping protein bands due to its limited resolving power. What appears to be a single band may actually be a mixture of different proteins. 2-D gel electrophoresis incorporates isoelectric focusing (IEF) in the first dimension and SDS-PAGE in the second dimension, leading to a separation by charge and size (O'Farrell, 1975). 2-D PAGE is a powerful technique for separating very complex protein preparations, resolving up to 10 000 proteins from mammalian tissues and other complex proteins (Klose and Kobalz, 1995; Celis et al., 1996; Yan et al., 1997). The proteins or protein isoforms of the present invention are identified by their isoelectric focusing point (pI) and their molecular weight (MW) in kilodaltons (kD). Accordingly, the present invention relates to a method as described above, characterized in that the level of protein or protein isoform is detected by isoelectric focusing followed by denaturing electrophoresis. Preferably, the step of denaturing electrophoresis uses sodium dodecyl sulfate polyacrylamide gel electrophoresis (SDS-PAGE).

Identification of some of the protein spots on 2-D gel electrophoresis that were altered between the studied groups is shown in Table 5.

As indicated above, the level of protein or protein isoform can also be detected by an immunoassay. As used herein, an “immunoassay” is an assay that utilizes an antibody to specifically bind to the antigen (i.e. the protein or protein isoform). The immunoassay is thus characterized by detection of specific binding of the proteins or protein isoforms to antibodies. Immunoassays for detecting proteins or protein isoforms may be either competitive or noncompetitive. Noncompetitive immunoassays are assays in which the amount of captured analyte (i.e. the protein or protein isoform) is directly measured. In competitive assays, the amount of analyte (i.e. the protein or protein isoform) present in the sample is measured indirectly by measuring the amount of an added (exogenous) analyte displaced (or competed away) from a capture agent (i.e. the antibody) by the analyte (i.e. the protein or protein isoform) present in the sample. In one competition assay, a known amount of the (exogenous) protein or protein isoform is added to the sample and the sample is then contacted with the antibody. The amount of added (exogenous) protein or protein isoform bound to the antibody is inversely proportional to the concentration of the protein or protein isoform in the sample before the exogenous protein or protein isoform is added. In one preferred “sandwich” assay, for example, the antibodies can be bound directly to a solid substrate where they are immobilized. These immobilized antibodies then capture the protein or protein isoform of interest present in the test sample. Other immunological methods include but are not limited to fluid or gel precipitation reactions, immunodiffusion (single or double), agglutination assays, immunoelectrophoresis, radioimmunoassays (RIA), enzyme-linked immunosorbent assays (ELISA), Western blots, liposome immunoassays (Monroe et al., 1986), complement-fixation assays, immunoradiometric assays, fluorescent immunoassays, protein A immunoassays or immunoPCR. An overview of different immunoassays is given in Wild D. (2001) and Ghindilis et al. (2002).

In a preferred embodiment, the level of the protein or protein isoform is determined by an immunoassay comprising at least the following steps:

(a) contacting the protein or protein isoform with an antibody that specifically recognizes the protein or protein isoform, under conditions suitable for producing an antigen-antibody complex; and

(b) detecting the immunological binding that has occurred between the antibody and the protein or protein isoform.

In another embodiment, the protein or protein isoform can be detected by a sandwich ELISA comprising the following steps:

(a) bringing said protein or protein isoform into contact with an antibody (primary antibody or capturing antibody) recognizing said protein or protein isoform, under conditions being suitable for producing an antigen-antibody complex;

(b) bringing the complex formed between said protein or protein isoform and said primary antibody into contact with another antibody (secondary antibody or detector antibody) specifically recognizing said protein or protein isoform, under conditions being suitable for producing an antigen-antibody complex;

(c) bringing the antigen-antibody complex into contact with a marker either for specific tagging or coupling with said secondary antibody, with said marker being any possible marker known to the person skilled in the art;

(d) possibly also, for standardization purposes, bringing the antibodies in contact with a purified protein or protein isoform reactive with both antibodies.

Advantageously, the secondary antibody itself carries a marker or a group for direct or indirect coupling with a marker.

The term “specifically recognizing”, “specifically binding with”, “specifically reacting with” or “specifically forming an immunological reaction with” refers to a binding reaction by the antibody to the protein or protein isoform which is determinative of the presence of the protein or protein isoform in the sample in the presence of a heterogeneous population of other proteins, other protein isoforms and/or other biologics. Thus, under the designated immunassay conditions, the specified antibody preferentially binds to a particular protein or protein isoform while binding to other proteins or protein isoforms does not occur in significant amounts.

Any antibody that recognizes the protein or protein isoform under examination can be used in the above method. Examples of antibodies that can be used in the detection of Apo E protein isoforms are listed in Table 7.

The present invention also relates to an antibody capable of specifically recognizing one of the following protein isoforms associated with one or more neurological diseases, among which are Alzheimer's disease, frontotemporal dementia, dementia with Lewy bodies, vascular dementia and/or depression:

Apo E: NPI 11, NPI 34, NPI 35, NPI 41, NPI 52, NPI 60, NPI 66, NPI 72, NPI 73, NPI 74, NPI 75, NPI 76m, NPI 77;

α-1-antitrypsin: NPI 1, NPI 42, NPI 43, NPI 44, NPI 59;

α-1-β glycoprotein: NPI 2, NPI 3, NPI 31, NPI 48;

Antithrombin-III: NPI 4;

Apo A-I: NPI 5, NPI 6, NPI 7, NPI 37, NPI 69, NPI 70, NPI 71;

Apo A-IV: NPI 8, NPI 9, NPI 10;

Apo J: NPI 12, NPI 13, NPI 14, NPI 15, NPI 16;

Gelsolin: NPI 17;

Haptoglobin: NPI 18;

Hemopexin: NPI 19, NPI 20;

Ig α-1 chain C region (heavy): NPI 21, NPI 22;

Kininogen: NPI 23;

Prostaglandin-H2 D-isomerase: NPI 24, NPI 25;

Transthyretin: NPI 26, NPI 27, NPI 28m;

Vitamin D-binding protein: NPI 29, NPI 30;

Zn-α-2-glycoprotein: NPI 33;

NPI 32, NPI 36, NPI 38, NPI 39, NPI 40, NPI 45, NPI 46, NPI 47, NPI 49, NPI 50, NPI 51, NPI 53, NPI 54, NPI 55, NPI 56, NPI 57, NPI 58, NPI 61, NPI 62, NPI 63, NPI 64, NPI 65, NPI 67, NPI 68.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. The basic immunoglobulin (antibody) structural unit is known to comprise a tetramer or dimer. Each tetramer is composed of two identical pairs of polypeptide chains, each pair having on “light” (about 25 kD) and on “heavy” chain (about 50-70 kD). The N-terminus of each chain defines a variable region of about 100 to 110 or more amino acids, primarily responsible for antigen recognition. The terms “variable light chain (V_{L})” and “variable heavy chain (V_{H})” refer to these variable regions of the light and heavy chains respectively.

Antibodies of the invention include, but are not limited to polyclonal, monoclonal, bispecific, humanized or chimeric antibodies, single variable fragments (ssFv), Fab fragments, F(ab′) fragments, fragments produced by a Fab expression library, anti-idiotypic antibodies and epitope-binding fragments of any of the above, provided that they retain the original binding properties. Also mini-antibodies and multivalent antibodies such as diabodies, triabodies, tetravalent antibodies and peptabodies can be used in a method of the invention. The preparation and use of these fragments and multivalent antibodies has been described extensively in International Patent Application WO 98/29442. The immunoglobulin molecules of the invention can be of any class (i.e. IgG, IgE, IgM, IgD and IgA) or subclass of immunoglobulin molecule. The NPI or a fragment or derivative thereof can be use as an immunogen to generate the antibodies of the invention which specifically bind such an immunogen. Various host animals can be immunized by injection with the native or a synthetic version of the NPI or the fragment or derivative of the NPI, including but not limited to rabbits, mice, rats, etc. Various adjuvants may be used to enhance the immunological response, depending on the host species, including but not limited to complete or incomplete Freund's adjuvant, a mineral gel such as aluminum hydroxide, surface active substances such as lysolecithin, pluronic polyol, a polyanion, a peptide, an oil emulsion, keyhole limpet hemocyanin, dinitrophenol, or an adjuvant such as BCG (bacille Calmette-Guérin) or Corynebacterium parvum. For the preparation of monoclonal antibodies, any technique which provides for the production of antibody molecules by continuous cell lines in culture may be used, including but not limited to the hybridoma technique developed by Kohler and Milstein (1975), the human B-cell hybridoma technique (Kozbor et al., 1983) or the EBV-hybridoma technique to produce human monoclonal antibodies (Cole et al., 1985). Screening for the desired antibody can be done by techniques known in the art such as ELISA. Selection of an antibody that specifically binds a first NPI but which does not specifically bind to a second NPI, can be made on the basis of positive binding to the first NPI and the lack of binding to the second NPI. Thus, in a particular embodiment, the present invention provides an antibody that binds with greater affinity (particularly at least 2-fold, more particularly at least 5-fold, still more particularly at least 10-fold greater affinity) to a first NPI than to a second NPI. In another preferred embodiment, the present invention provides an antibody that binds with greater affinity (particularly at least 2-fold, more particularly at least 5-fold, still more particularly at least 10-fold greater affinity) to a first NPI than to a second NPI of the same protein. These antibodies are also called anti-NPI antibodies.

While various antibody fragments are defined in terms of enzymatic digestion of an intact antibody with papain, pepsin or other proteases, those skilled in the art will appreciate that such antibody fragments as well as full size antibodies may be synthesized either chemically or by utilizing recombinant DNA methodology. Thus, the term antibody, as used herein also includes antibodies and antibody fragments either produced by the modification of whole antibodies or synthesized using recombinant DNA methodologies. The humanized versions of the mouse monoclonal antibodies are also made by means of recombinant DNA technology, departing from the mouse and/or human genomic DNA sequences coding for H and L chains or from cDNA clones coding for H and L chains. Alternatively the monoclonal antibodies used in the method of the invention may be human monoclonal antibodies. The term ‘humanized antibody’ means that at least a portion of the framework regions of an immunoglobulin is derived from human immunoglobulin sequences.

The antibodies used in the method of the present invention may be labeled with an appropriate label. The particular label or detectable group used in the assay is not a critical aspect of the invention, so long as it does not significantly interfere with the specific binding of the antibody used in the assay. The detectable group can be any material having a detectable physical or chemical property. Such detectable labels have been well developed in the field of immunoassays and, in general, almost any label used in such methods can be applied to the method of the present invention. Thus, a label is any composition detectable by spectroscopic, photochemical, biochemical, immunochemical, electrical, radiological, optical, or chemical means. Useful labels in the present invention include, but are not limited to, magnetic beads (e.g. Dynabeads™), fluorescent dyes (e.g. fluorescein isothiocyanate, texas red, rhodamine), radiolables (e.g. ³H, ¹²⁵I, ³⁵S, ¹⁴C, or ³²P), enzymes (e.g. horseradish peroxidase, alkaline phosphatase, and others commonly used in an ELISA), and calorimetric labels such as colloidal gold, colored glass or plastic (e.g. polystyrene, polypropylene, latex, etc.) beads.

The label may be coupled directly or indirectly to the desired component or the assay according to methods well known in the art. As indicated above, a wide variety of labels may be used, with the choice of label depending on the sensitivity required, the ease of conjugation with the compound, stability requirements, the available instrumentation, and disposal provisions. Non-radioactive labels are often attached by indirect means. Generally, a ligand molecule (e.g. biotin) is covalently bound to the antibody. The ligand then binds to an anti-ligand (e.g. streptavidin) molecule, which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, a haptenic or antigenic compound can be used in combination with an antibody. The antibodies can also be conjugated directly to signal generating compounds, for example, by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umberlliferone, etc. Chemiluminescent compounds include luciferin, and 2,3-dihydrophthalazinediones, for example, luminol. A review of other labeling or signal producing systems is available in U.S. Pat. No. 4,391,904.

Means for detecting labels are well known in the art. Thus, for example, where the label is a radioactive label, means for detection include a scintillation counter or photographic film, as in autoradiography. Where the label is a fluorescent label, it may be detected by exciting the fluorophore with the appropriate wavelength of light and detecting the resulting fluorescence. The fluorescence may be detected visually, by means of a photographic film, by the use of electronic detectors such as charge coupled devices (CCDs) or photomultipliers and the like. Similarly, enzyme labels may be detected by providing the appropriate substrates for the enzyme and detecting the resulting reaction product. Finally simple colorimetric labels may be detected simply by observing the color associated with the label.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

The method for the in vivo detection of the level of a protein or a protein isoform in a mammal comprises the steps of determining the level of said protein or protein isoform in said mammal and comparing it with a previously defined level range characteristic for control mammals, or for mammals with AD, FTD, DLB, VAD and/or depression, or with a previously defined cut-off value suitable for differentiating two of those neurological diseases. In an embodiment of the invention, the level of protein or protein isoform can be determined by in vivo imaging. The level of protein or protein isoform can be determined in situ by non-invasive methods including but not limited to brain imaging methods described by Arbit et al. (1995), Tamada et al. (1995), Wakabayashi et al. (1995), Huang et al. (1996), Sandrock et al. (1996), Mariani et al. (1997). These in vivo imaging methods may allow the localization and quantification of the protein or protein isoform, for example, by use of labeled antibodies (as described above) specifically recognizing said protein or protein isoform. Other methods for in vivo detection of proteins or protein isoforms are described by Poduslo et al. (2002), Small (2002), and Petrella et al. (2003).

The invention also provides diagnostic kits comprising an anti-NPI antibody. The invention thus provides a diagnostic kit for the screening, diagnosis and/or prognosis in a mammal of one or more neurological diseases, among which are Alzheimer's disease, frontotemporal dementia, dementia with Lewy bodies, vascular dementia and/or depression, for identifying a mammal at risk of developing one or more neurological diseases, among which are Alzheimer's disease, frontotemporal dementia, dementia with Lewy bodies, vascular dementia and/or depression, or for monitoring the effect of therapy administered to a mammal having one or more neurological diseases, among which are Alzheimer's disease, frontotemporal dementia, dementia with Lewy bodies, vascular dementia and/or depression, characterized that said kit comprises an anti-NPI antibody. The present invention thus also provides a diagnostic kit for the differential diagnosis in a mammal of different neurological diseases, among which are Alzheimer's disease, frontotemporal dementia, dementia with Lewy bodies, vascular dementia and/or depression, characterized that said kit comprises an anti-NPI antibody. A preferred kit for carrying out the method of the invention comprises:

an antibody (primary antibody) which forms an immunological complex with the protein or protein isoform to be detected;

a monoclonal antibody (secondary antibody) which specifically recognizes the protein or protein isoform to be detected;

a marker either for specific tagging or coupling with said secondary antibody;

appropriate buffer solutions for carrying out the immunological reaction between the primary antibody and the protein or protein isoform, between the secondary antibody and the primary antibody-protein or -protein isoform complex and/or between the bound secondary antibody and the marker;

possibly, for standardization purposes, a purified protein or protein isoform.

As it is known that the occurrence of some neurological diseases in a person is more frequent at a certain age, age-related kits can be prepared comprising antibodies that recognize specific proteins or protein isoforms that are associated with one or more neurological diseases that occur more frequent at that specific age.

Accordingly, the present invention provides an antibody or a kit as defined above, for use in the screening, diagnosis or prognosis in a mammal of Alzheimer's disease, frontotemporal dementia, dementia with Lewy bodies, vascular dementia and/or depression, for identifying a mammal at risk of developing Alzheimer's disease, frontotemporal dementia, dementia with Lewy bodies, vascular dementia and/or depression, or for monitoring the effect of therapy administered to a mammal having Alzheimer's disease, frontotemporal dementia, dementia with Lewy bodies, vascular dementia and/or depression.

The present invention also provides an antibody or a kit as defined above for use in the differential diagnosis in a mammal of Alzheimer's disease, frontotemporal dementia, dementia with Lewy bodies, vascular dementia and/or depression.

Also included in the present invention is the use of an antibody as defined above for the preparation of a kit for the screening, diagnosis or prognosis in a mammal of Alzheimer's disease, frontotemporal dementia, dementia with Lewy bodies, vascular dementia and/or depression, for identifying a mammal at risk of developing Alzheimer's disease, frontotemporal dementia, dementia with Lewy bodies, vascular dementia and/or depression, or for monitoring the effect of therapy administered to a mammal having Alzheimer's disease, frontotemporal dementia, dementia with Lewy bodies, vascular dementia and/or depression.

Also included in the present invention is the use of an antibody as defined above for the preparation of a kit for the differential diagnosis in a mammal of Alzheimer's disease, frontotemporal dementia, dementia with Lewy bodies, vascular dementia and/or depression.

The present invention also provide methods of screening for agents that interact with and/or modulate (have a stimulatory or inhibitory effect on) the expression or activity of a protein or protein isoform associated with one or more neurological diseases, among which are Alzheimer's disease, frontotemporal dementia, dementia with Lewy bodies, vascular dementia and/or depression, said method comprising:

(a) contacting said protein or protein isoform or a portion of said protein or protein isoforn with said agent; and

(b) determining whether or not said agent interacts with and/or modulates the expression or activity of said protein or protein isoform or said portion of the protein or protein isoform.

Candidate agents or test agents include, but are not limited to, nucleic acids (DNA or RNA), carbohydrates, lipids, proteins, peptides, small molecules and other drugs. Agents can be obtained using any of the numerous suitable approaches in combinatorial library methods known in the art, including: biological libraries, spatially addressable parallel solid phase or solution phase libraries, synthetic library methods requiring deconvolution, the “one-bead one-compound” library method and synthetic library methods using affinity chromatography selection. Library compounds may be presented in solution, on beads, chips, bacteria, spores, plasmids, or phage.

In one embodiment, a protein or protein isoform is identified in a cell-based assay system. In accordance with this embodiment, cells expressing the protein or protein isoform or a fragment thereof are contacted with the candidate agent or a control compound and the ability of the candidate agent to interact with the protein or protein isoform or to modify the biological behaviour of the cell is measured. The cell can be of prokaryotic origin (e.g. E. coli) or of eukaryotic origin (e.g. yeast or mammalian). The protein or protein isoform or the candidate agent can be labeled (described above), to enable detection of an interaction between the protein or protein isoform and the candidate agent. Interaction can then be detected by flow cytometry, by scintillation assay, by immunoprecipitation, by Western blot analysis, by its ability to modify or by other means.

In another embodiment, agents that interact with a protein or protein isoform are identified in a cell-free assay system. In accordance with this embodiment, a native, chemically synthesized or recombinant protein or protein isoform or a fragment thereof is contacted with the candidate agent or a control compound and the ability of the candidate agent to interact with the protein or protein isoform is determined. Preferably, the protein or protein isoform or fragment thereof is first immobilized by, for example, contacting the protein or protein isoform or the fragment thereof with an immobilized antibody that specifically recognizes said protein or protein isoform or said fragment thereof, or by contacting the protein or protein isoform or the fragment thereof with a surface designed to bind proteins.

In another embodiment, a cell-based assay system is used to identify agents that bind to or modulate the activity of a protein, such as an enzyme, or a biologically active portion thereof, which is responsible for the production or degradation of a protein or protein isoform or which is responsible for the post-translational modification of a protein or protein isoform. In a primary screen, a plurality (e.g., a library) of compounds are contacted with cells that naturally or recombinantly express: (i) a protein or protein isoform or a biologically active fragment thereof; and (ii) a protein that is responsible for processing of the protein or protein isoform or the fragment thereof in order to identify compounds that modulate the production, degradation, or post-translational modification of the protein or protein isoform. If desired, compounds identified in the primary screen can then be assayed in a secondary screen against cells naturally or recombinantly expressing the specific protein or protein isoform of interest. The ability of the candidate compound to modulate the production, degradation or post-translational modification of a protein or protein isoform can be determined by methods known to those skilled in the art, including without limitation, flow cytometry, a scintillation assay, immunoprecipitation, and Western blot analysis. In another embodiment, agents that competitively interact with (i.e., bind to) a protein or protein isoform or a fragment thereof are identified in a competitive binding assay. In accordance with this embodiment, cells expressing a protein or protein isoform or a fragment thereof are contacted with a candidate compound and a compound known to interact with the protein or protein isoform or the fragment thereof. The ability of the candidate compound to competitively interact with the protein or protein isoform or the fragment thereof is then determined.

Alternatively, agents that competitively interact with (i.e., bind to) a protein or protein isoform or a fragment thereof are identified in a cell-free assay system by contacting a protein or protein isoform or a fragment thereof with a candidate compound and a compound known to interact with the protein or protein isoform or the fragment thereof. As stated above, the ability of the candidate compound to interact with a protein or protein isoform or a fragment thereof can be determined by methods known to those skilled in the art. These assays, whether cell-based or cell-free, can be used to screen a plurality (e.g., a library) of candidate compounds.

In another embodiment, agents that modulate (i.e., upregulate or downregulate) the expression of a protein or protein isoform are identified by contacting cells (e.g., cells of prokaryotic origin or of eukaryotic origin) expressing the protein or protein isoform with a candidate compound or a control compound (e.g., phosphate buffered saline (PBS)) and determining the expression of the protein or protein isoform or mRNA encoding the protein or protein isoform. The level of expression of a selected protein or protein isoform or mRNA encoding the protein or protein isoform in the presence of the candidate compound is compared to the level of expression of the protein or protein isoform, or mRNA encoding the protein or protein isoform in the absence of the candidate compound (e.g., in the presence of a control compound). The candidate compound can then be identified as a modulator of the expression of the protein or protein isoform, based on this comparison. For example, when expression of the protein or protein isoform or mRNA encoding the protein or protein isoform is significantly greater in the presence of the candidate compound than in its absence, the candidate compound is identified as a stimulator of expression of the protein or protein isoform or mRNA encoding the protein or protein isoform. Alternatively, when expression of the protein or protein isoform or mRNA encoding the protein or protein isoform is significantly less in the presence of the candidate compound than in its absence, the candidate compound is identified as an inhibitor of the expression of the protein or protein isoform or mRNA encoding the protein or protein isoform. The level of expression of a protein or protein isoform or the mRNA that encodes it can be determined by methods known to those skilled in the art based on the present description. For example, mRNA expression can be assessed by Northern blot analysis or RT-PCR, and protein levels can be assessed by Western blot analysis.

In another embodiment, agents that modulate the activity of a protein or protein isoform are identified by contacting a preparation containing the protein or protein isoform, or cells (e.g., prokaryotic or eukaryotic cells) expressing the protein or protein isoform with a test compound or a control compound and determining the protein or protein isoform. The activity of a protein or protein isoform can be assessed by different methods. The induction of a cellular signal transduction pathway of the protein or protein isoform (e.g., intracellular Ca²⁺, diacylglycerol, IP3, etc.) can be detected. In other cases the catalytic or enzymatic activity of the target on a suitable substrate can be detected. The induction of a reporter gene (e.g., a regulatory element that is responsive to a protein or protein isoform and is operably linked to a nucleic acid encoding a detectable marker, e.g., luciferase) can be measured. A cellular response, for example, cellular differentiation, or cell proliferation, as the case may be, can be detected. Based on the present description, techniques known to those skilled in the art can be used for and detecting these activities (see, e.g., U.S. Pat. No. 5,401,639). The candidate agent can then be identified as a modulator of the activity of a protein or protein isoform by comparing the effects of the candidate compound to the control compound. Suitable control compounds include phosphate buffered saline (PBS) and normal saline (NS).

In another embodiment, agents that modulate (i.e., upregulate or downregulate) the expression, activity or both the expression and activity of a protein or protein isoform are identified in an animal model. Examples of suitable animals include, but are not limited to, mice, rats, rabbits, monkeys, guinea pigs, dogs and cats. Preferably, the animal used represents a model of Alzheimer's disease (for example: animals that express human familial Alzheimer's disease (FAD) amyloid precursor protein (APP), animals that overexpress human wild-type APP, animals that overexpress β-amyloid₍₁₋₄₂₎ (βA), animals that express FAD presenillin-1 (PS-1)), or a model for another neurological disease such as FTD, DLB, VAD or depression. In accordance with this embodiment, the test compound or a control compound is administered (e.g., orally, rectally or parenterally such as intraperitoneally or intravenously) to a suitable animal and the effect on the expression, activity or both expression and activity of the protein or protein isoform is determined. Changes in the expression of a protein or protein isoform can be assessed by any suitable method described above, based on the present description.

WO 01/75454 enumerates other techniques and scientific publications describing suitable assays for detecting or quantifying enzymatic, modulating and/or binding activities of a protein or protein isoform or a fragment thereof. Each such reference is hereby incorporated in its entirety.

Throughout this specification and the claims which follow, unless the context requires otherwise, the word “comprise”, and variations such as “comprises” and “comprising”, will be understood to imply the inclusion of a stated integer or step or group of stated integers or steps but not to the exclusion of any other integer or step or group of integers or steps.

The present invention will now be illustrated by reference to the following examples that set forth particularly advantageous embodiments. However, it should be noted that these examples are illustrative and cannot be construed as to restrict the invention in any way.

### EXAMPLES

### Example 1

### Materials and Methods

1.1. Patients and CSF Samples

A study was performed on CSF samples obtained from the Department of Clinical Neuroscience, Sahlgren's University Hospital, Mölndal, Sweden. An overview of all the CSF samples that were analyzed is given in Table 1. The neurological diseases from which the patients (from whom the CSF samples were taken) were suffering, are also indicated in Table 1 (diagnosis). The following clinical criteria were used for the diagnosis of these neurological diseases. For the diagnosis of Alzheimer's disease, the NINCDS-ADRDA criteria (McKhann et al., 1984) were used. For the diagnosis of dementia with Lewy bodies, the criteria according to McKeith (McKeith et al., 1996) were used. For the diagnosis of frontotemporal dementia, the Lund-Manchester guidelines (The Lund and Manchester Groups, 1994) were used. Vascular dementia was diagnosed according to the National Institute of Neurological Disorders and Stroke-Association Internationale pour 1a Recherche et 1'Enseignement en Neuroscience criteria (NINDS-AIREN; Roman et al., 1993). Diagnosis of depression was made either according the DSM-IV criteria or the ICD-10 criteria (DSM-IV and WHO International Classification of Diseases, 10^{th} Revision). Patients with non-dementing conditions (controls) were also included in the study. Table 1 also includes data on patient gender, age, Minimal Mental State Examination score, the levels of tau and β-amyloid₍₁₋₄₂₎ (Ab42) as well as the albumin ratios present in the CSF samples.

1.2. Sample Preparation

CSF samples, each in duplicate, were precipitated with 2 volume equivalents of ice-cold acetone during a 2-hour incubation at −20° C. The protein pellets (corresponding to 300 μl CSF (±150 μg protein) were collected by centrifugation and resolubilized in rehydration buffer containing 8 M urea, 2% w/v CHAPS, 2% w/v IPG buffer pH 4.5-5.5, and 1.8% dithiothreitol (DTT) to yield the desired amount of protein in a volume that was loaded on Immobiline DryStrips pH 4.5-5.5. In-gel rehydration was performed at room temperature over a period of 18 hours.

In a second experiment, 600 μl undiluted CSF was fractionated by means of ABDstab dimer Sepharose to remove albumin, and by rProtein A Sepharose affinity chromatography to deplete IgG (Amersham Pharmacia Biotech, Uppsala, Sweden). ‘Halt’ protease inhibitor cocktail (Pierce, Rockford, Ill., USA) was added to undiluted CSF prior to prefractionation as well as to the pool of eluted protein (±150 μg CSF protein; albumin and IgG excluded), followed by incubation with 100 mM DTT (1 h, room temperature). Further sample preparation was carried out as described above.

The amount of protein in the CSF samples was determined with a bicinchoninic acid (BCA) protein assay kit (Pierce).

1.3. Isoelectric Focusing (IEF) and 2-D PAGE

Separation of proteins according to their isoelectric points (first dimension) was performed at 115,000 Vh (49 h, 18° C.) in the first, and at 71,275 Vh (28 h, 18° C.) in the second experiment, using a Multiphor II with a Pharmacia LKB Multidrive XL power supply (Amersham Pharmacia Biotech) on immobilized, narrow-area pH gradients at pI range 4.5-5.5 (Cat.no. 17-6001-85 Amersham Pharmacia Biotech) used as described in Sample preparation. The theoretical pI values for all protein spots were approximated from drawings supplied with IPG narrow-area strips. To separate proteins by molecular weight, the ISO-DALT 2-D PAGE system (Amersham Pharmacia Biotech) was used, allowing simultaneous 10-gel runs (gel size 20 cm). For this purpose, 12.5% Tris-glycine home-cast gels were used with a crosslinker ratio of 15:1 (30% acrylamide −2% bis-acrylamide) (Bio-Rad Laboratories, Hercules, Ca., USA).

The theoretical molecular mass values (EXPASY) of the α-1-β glycoprotein (80,046 kDa), α-1-antitrypsin (57,197 kDa), apolipoprotein A-IV (43,374 kDa) and the in-house determined molecular mass of transthyretin (13,761 kDa) were logarithmically interpolated to the protein spots by use of the PDQuest 2-D Gel Analysis Software suite (Bio-Rad). Prior to staining, gels were fixed in 10% methanol −7% acetic acid solution, twice for 30 min. The spots were visualized with the fluorescent dye ‘SYPRO Ruby’ (Bio-Rad). After fixation in 10% methanol—7% acetic acid, gels were stained for 3 hours or overnight, and then destained in 10% methanol −7% acetic acid. The gels were digitized with the Image Master VDS camera (Amersham Pharmacia Biotech) in the first, and with the ProXPRESS™ imaging system (PerkinElmer Life Sciences, Boston, Mass., USA) in the second experiment. The gel images were processed using the PDQuest 2-D Gel Analysis Software suite (Bio-Rad) and HT Analyser Software (Genomic Solutions, Ann Arbor, Mich., USA) in the first and second experiments, respectively. The protein spots from different gels were matched, and their spot volumes were determined.

1.4. Protein Identification

Selected protein spots were digested with trypsin in situ. The eluted peptides were sequenced by electrospray mass spectrometry. NanoLC-ESI-TOF-MS and tandem MS with column switching was used in the instrumental settings as previously described (Raymackers et al., 2000). Briefly, samples were injected on a 0.3 mm×1 mm Pep-Map C18 precolumn (LC Packings, San Francisco, Calif., USA) followed by back-flushing on a nano-PepMap 0.075 mm×150 mm column (LC Packings) and separation of the bound peptides using an appropriate gradient solvent delivery system (0.1% formic acid in water, then 80% acetonitrile/0. 1% formic acid in water) at a flow rate of 230 nl/min. The column was directly coupled to the Q-TOF (Micromass, Wythenshawe, UK), with mass spectrometer software Masslynx 3.4 (Micromass) directing automatic MS to tandem MS switching. The generated MS/MS spectra were automatically searched against human databases. MS/MS spectra that remained uninterpreted after this search were sequenced manually and screened for protein using in-house databases.

1.5. Antibodies and Western Blot Analysis

For the determination of total number of protein isoforms related to a particular protein, the immunoblot method was used. Briefly, CSF samples were loaded on IPG strips: 100 μL on 7-cm strips, or 300 μL on the 18-cm strips (Amersham Pharmacia Biotech). IEF was performed at 7,474 Vh and at 71,275 Vh for the short and long strips, respectively. Samples separated by SDS-PAGE [12.5 or 4% -20% (w/v) gel] were electroblotted on nitrocellulose or polyvinylidene fluoride membranes for immunologic detection. Transfer was performed in Tris-Gly buffer (25 mM Tris, 192 mM glycine and 15% methanol) at a current of 1 mA/cm² of gel surface. The SuperSignal West Dura Extended Duration Substrate System (Pierce) was used for detection. The antibodies used in the Western blot assay are listed in Table 7.

### 1.6. Data Analysis

Differences in protein expression between disease groups were explored by pairwise comparison of disease groups on a spot-by-spot basis (pairs are disease groups). Because a spot was not always observed in all patients or replicates, differences were assessed at two levels. First, a qualitative analysis was performed on the number of spots observed per disease group using Fisher's exact test to determine whether a spot occurs more frequently in a given group. For the second, quantitative analysis, the subset of data with a quantitative spot intensity value was logarithmically transformed and analyzed by the t-test. This analysis reveals whether an observed spot has a higher intensity in a given group when observed. Given the explorative character of this study, no correction for multiple testing was applied, and all tests were done at a significance level of p=0.05.

Assessment of the correlation between the albumin ratio and protein expression was performed on a data set with control, AD, and FTD CSF samples. Because limited data were available for assessing normality, the significance of the Pearson and the Spearman rank-order correlations was taken into account.

### Example 2

### Comparison of the Protein Profiles in CSF Samples Obtained from Patients Suffering from AD, from FTD, from VAD and Control Patients

2.1. Master 2-D Map and Protein Identification by MS

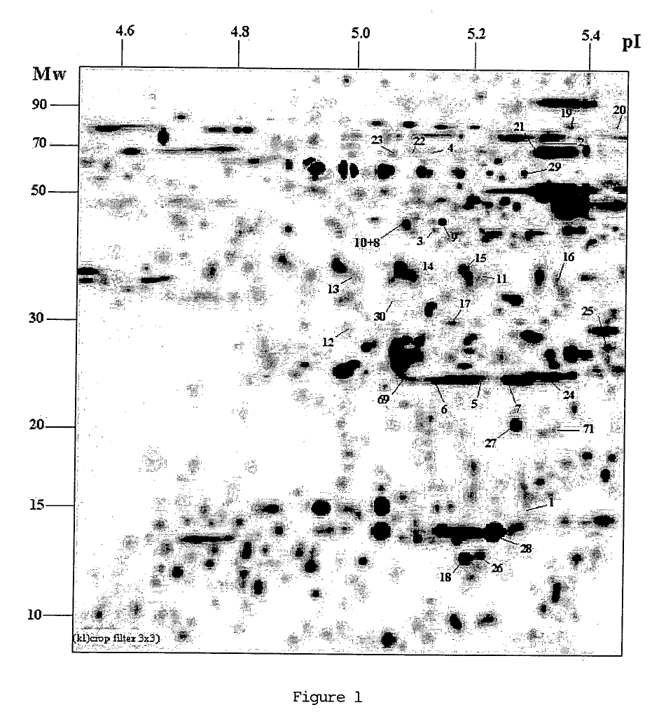

In the first experiment, 2-D differential analysis was performed on 18 CSF samples derived from 6 patients with Alzheimer's disease (AD 1-AD 6), 6 with FTD (FTD 1-FTD 6), and 6 age-matched healthy controls (C 1-6) (Table 1). A total of 622 protein spots were matched among the 36 gels (for each CSF sample in duplicate). The changes in protein spot intensities, as calculated by PDQuest processing, indicated that out of 622 spots, approximately 100 distinct protein spots were differentially regulated and independently related to AD, FTD, or to controls. The high resolution and reproducible patterns of the 2-D protein gels (12.5% PAGE, narrow range pI 4.5-5.5) allowed detection of quantitative and qualitative differences in the protein composition of the CSF samples. Qualitative and quantitative data are listed in Table 2 as fold differences and/or counts between groups (counts meaning a number of data points, counted in analyzed groups for each protein isoform). All matched protein isoforms among AD, FTD, and control are shown on the 2-D master map (FIG. 1). Out of these, for 44 protein spots, a partial protein sequence was obtained. In combination with in-house database searches the protein species were identified (Table 5). With respect to the type of proteins detected, three major groups of differentially expressed proteins were defined: proteins influenced by blood-brain-barrier integrity, CSF-specific proteins such as prostaglandin-H2 D-isomerase, and apolipoproteins.

2.2. Proteins Influenced by Blood-brain-barrier Integrity

The expression levels of several blood-derived proteins were mainly up-regulated in the CSF samples of FTD patients. In this study those patients generally have a higher albumin ratio than AD or controls, we suspected that these differences resulted from an impaired blood-brain-barrier. We indeed noted that vitamin D-binding protein, hemopexin, haptoglobin, antithrombin III, transthyretin, and α-1-β glycoprotein, which might have originated from the blood, were often up-regulated in FTD CSF, although most of these differences were of a qualitative nature. Nine of these protein isoforms were selected to ascertain any possible correlation with the albumin ratio. However, a positive correlation between abundance and albumin ratio was only found for the one isoform of vitamin D-binding protein (NPI 29), accounting for 35% of the variation observed between FTD and AD. This indicated that the albumin ratio could be only partially responsible for the observed differences in protein expression.

Significantly up-regulated proteins in FTD samples were found for α-1-β glycoprotein (NPI 2, 3) and for transthyretin (NPI 27, 28m) versus AD, and for antithrombin III (NPI 4), haptoglobin (NPI 18), hemopexin (NPI 19, 20), and transthyretin (NPI 28m) compared with controls. By contrast, transthyretin NPI 26 was increased in the AD compared with the FTD patients.

2.3. Prostaglandin-H2 D-isomerase

Prostaglandin-H2 D-isomerase, abundant in the CSF and at very low concentrations in the serum (16.6 mg/L versus 0.49 mg/L, respectively) is considered to be a CSF-specific protein (Reiber, 2001). As prostaglandin-H2 D-isomerase NPIs 24 and 25 were absent in all but one of the FTD and AD gels, only qualitative values were determined. The expression of prostaglandin-H2 D-isomerase NPI 25 was down-regulated in AD compared with FTD and controls.

2.4. Changes in Apolipoprotein Levels

The majority of the altered CSF protein profiles belonged to the apolipoprotein group. For Apo J, expression levels of 5 protein species were changed: in particular, spots NPI 13, 14, 15, and 16 were down-regulated in AD versus FTD patients. Apo J NPI 12 was down-regulated only in FTD versus controls. Three out of four protein species determined by MS as Apo A-IV were detectably altered in the CSF. The most significant of these proteins was the A-IV NPI 8, which was down-regulated in AD and controls compared with FTD.

Among the six altered Apo A-I spots, the expression of the Apo A-I NPI 5 was particularly increased in AD compared with FTD. Finally, Apo E was detected in the 2-D gel in two molecular mass forms: as an entire length or as a truncated isoform. Only the entire length isoform NPI 11 was significantly down-regulated in AD versus FTD samples.

2.5. Additional VAD and FTD Samples

Because previous experiments pointed to the differential expression of Apo A-I and Apo E, further validation of these trends was pursued by analyzing 4 additional FTD and 4 VAD CSF samples (B1-8; Table 1). These 4 FTD samples were assessed together with the previous 6 FTD samples, and compared with AD samples. The 4 VAD CSF were also compared with the previously analyzed AD samples (Tables 3 and 4). Taken together, the apolipoproteins showed the same patterns for FTD CSF as found in the previous experiment. For VAD CSF, two truncated Apo E isoforms (NPI 74 and NPI 76m) were up-regulated versus AD.

### Example 3

### Comparison of the Protein Profiles in CSF Samples Obtained from Patients Suffering from AD with the Protein Profiles in CSF Samples Obtained from Patients Suffering from Depression, after Prefractionation

In a first experiment, after matching and comparing the gels, 87 different spots on the gel were identified by nano-LC-MS/MS. In these spots, 21 proteins were identified, implying that an average of 4 spots per protein were present. However, 18 of the 87 proved to be albumin (breakdown) spots, while the presence of albumin together with another protein was identified in another 21 spots. Even when the main albumin spot was not present on the zoom gel pI 4.5 to 5.5, albumin severely interfered with the differential analysis of CSF proteins.

To increase electrophoretic resolution and enhance detection sensitivity for low-abundant proteins, CSF prefractionation was included in the second experiment. In this second differential experiment in which 6 Alzheimer samples were compared with 6 depression samples, the albumin- and IgG-depleted CSF was analyzed using the same nano-LC-MS/MS method. In total, we identified 17 different proteins in 54 spots without any presence of an albumin peptide.

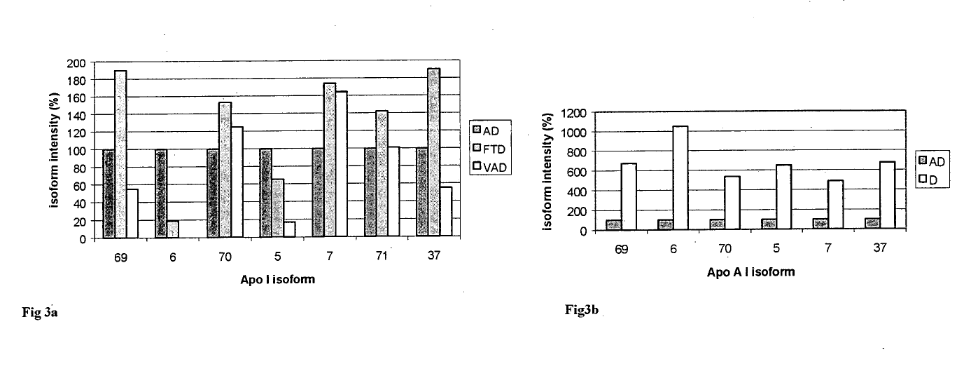

CSF samples from 6 AD patients (AD 7-AD 12) were compared with those from 6 depression patients (D 1-D 6). The total number of protein spots determined in this experiment was 1003, of which 41 were differentially regulated between the two groups. Due to the CSF depletion of albumin and IgG, the electrophoretic resolution of the gel segments was improved. The breakdown products of these two proteins were also successfully removed, providing better spot detection in the low molecular weight area of the gel. The levels of protein isoforms detected between AD and depression samples (Table 2) confirmed the results of the first experiment (AD/FTD/C), which again showed CSF apolipoproteins to be the most prominently altered among AD patients. In particular, additional truncated Apo E spots were detected. Apo A-I isoforms followed the same patterns as in the previous analysis of total CSF (FIG. 3). Moreover, the differences detected were more even pronounced than in the previous evaluation (Table 2, comparison of AD versus D; FIGS. 3a and 3b). This could be due to a higher dynamic range of the ProXPRESS™ imaging system (PerkinElmer Life Sciences) used in the second experiment.

Other protein isoforms that were differentially regulated in patients suffering from AD versus patients suffering from depression are indicated in Table 6.

### Example 4

### Comparison of the Level of Different Apolipoprotein A-I Isoforms in CSF Samples Obtained from Patient Suffering from AD, from FTD, from VAD, from Depression and Controls

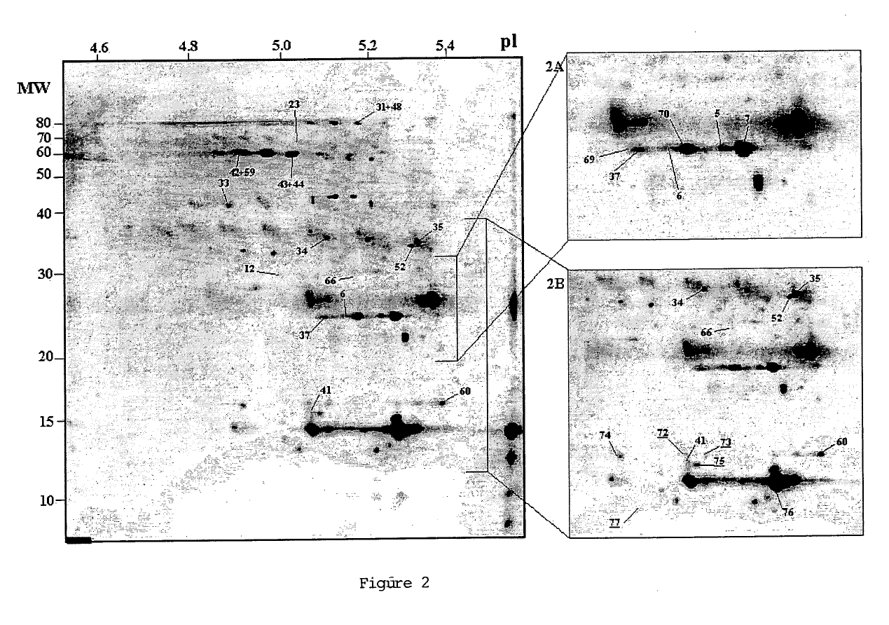

Apolipoprotein A-I patterns were examined by proteomic screening procedures in 38 patients based on the two experiments. First, we compared the CSF proteome of 6 AD patients, 6 FTD patients, and 6 healthy controls (Table 3). In addition, all identified Apo A-I spots were matched between all CSF samples. The Apo A-I isoforms 5, 6, 7, 37, 69, 70, and 71 were differentially and significantly regulated (Tables 2 and 3). The most prominent isoform was Apo AI NPI 7, which was significantly down-regulated in AD compared with FTD and VAD. There, was also a trend towards down-regulation in AD versus depressed patients (Table 2 and 3; FIG. 2; FIGS. 3a and 3b). By contrast, NPI 5 levels were up-regulated in AD compared with FTD and VAD patients (p<0.03 and p<0.004, respectively). Taken together, the most abundant Apo A-I isoforms (7, 70) were down-regulated in all AD samples when compared with their different contrast groups (FIGS. 2 and 3).

### Example 5

### Comparison of the Level of Different Apolipoprotein E Isoforms in CSF Samples Obtained from Patients Suffering from AD, from FTD, from VAD, from Depression and Controls

The gel area where apolipoprotein E isoforms were previously detected became more accessible for spot identification after serum albumin and IgG were removed from CSF. In the two experiments performed, a total of 13 Apo E isoforms were identified, of which 5 were full-length Apo E isoforms (NPI 11, 34, 35, 52, 66), and 8 were truncated fragments (NPI 72, 73, 74, 75, 41, 76, 77, 60) (Table 4). All 5 full-length isoforms and 4 truncated isoforms (NPI 74, 41, 76, 60) were significantly altered (p<0.05) between dementia and non-dementia controls (Table 2). The expression levels of 3 full-length isoforms (NPI 34, 35, 52) were significantly decreased in Alzheimer's when compared with depression patients (Table 4).

The difference in protein spot intensity observed for the Apo E NPI 34 was decreased up to 62 times in the AD patient group versus depression, by far the largest difference found in our study (p<0.002). Two other full-length Apo E isoforms, NPI 35 and NPI 52, followed the same pattern with decreases in the order of 24-fold (both p<0.04). For all Apo E isoforms detected, differences in expression levels were compared based on 6 AD (AD 1-AD 6), all 10 FTD, and 4 VAD samples (Table 4). Most isoforms were only detected in the second experiment (6 AD versus 6 D), but mostly not significantly different compared with values determined in the first experiment (Table 4). In the comparisons between AD and VAD patients, decreased levels of the truncated Apo E NPI 74 and 76 were correlated with the diagnosis of AD (p<0.045 and p<0.03, respectively, corresponding to 2.8- and 1.5-fold decreases in AD).

### Example 6

### Detection of Apo E Isoforms by Immunoblotting

6.1. CSF Samples

A study was performed on a pool of random CSF samples (without diagnose) obtained from the Department of Clinical Neuroscience, Sahlgren's University Hospital, Mölndal, Sweden.

6.2. Sample Preparation

The CSF pool was precipitated overnight with 2 volumes equivalent of ice-cold acetone, at −20° C. The protein pellets were collected by centrifugation and resolubilized in rehydratation buffer, containing 8 M urea, 2% w/v CHAPS, 2% IPG buffer pH 4.5-5.5 and 1.8% 1M DTT to yield the desired protein amount in a volume that was loaded on the Immobiline DryStrips pH 4.5-5.5 (Amersham Biosciences, Uppsala, Sweden). Also proteinase inhibitors (Pierce) were added to the rehydratation buffer (1/100). In gel rehydratation was performed at room temperature overnight.

6.3. IEF and 2D-PAGE

The separation of the proteins by their isoelectric point (first dimension) was performed at 71275 Vhr. (28 hours at 18° C.) using a Multiphor II, with a Pharmacia LKB Multidrive XL powersupply (Amersham Pharmacia Biotech, Uppsala, Sweden) on immobilized pH gradients (IPG) prepared as described above. The proteins were separated by molecular weight on the Protean II system (Bio-Rad Laboratories, Hercules, Calif., US).

6.4. Western Blot Analysis

The gels were blotted on Nitrocellulose membranes at 1.5 mA/cm², for 1 hour, using semi-dry blotting (Amersham Biosciences) and a discontinu buffer system.

6.5. Immunodetection and Chemiluminiscence

The membranes were blocked for 1½ hour in blocking buffer. Incubation with the first antibody (13F4B5, dilution: 1 μg/ml) occurred overnight. Membranes were washed 3 times during 10 minutes and incubated with the second antibody (1:100.000) during 1 hour. After washing 3 times during 10 minutes, signals were detected with ECL chemiluminiscent substrate (Amersham Biosciences) on film. Then, the blot was washed for 1 hour and couloured with colloidal gold stain.

6.6. Comparison of 2-D Gels and Blots

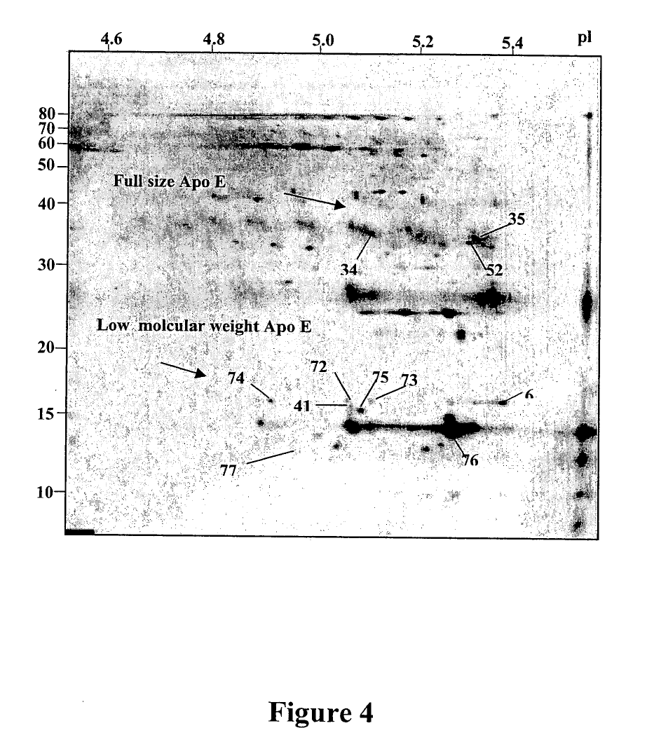

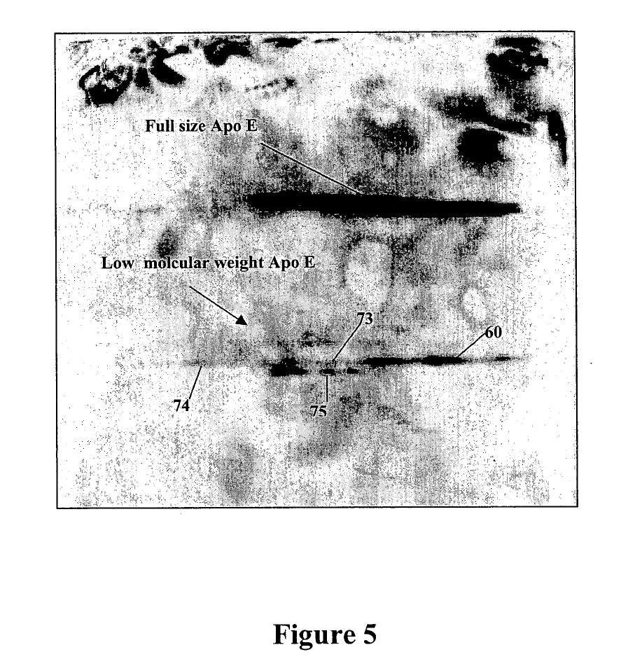

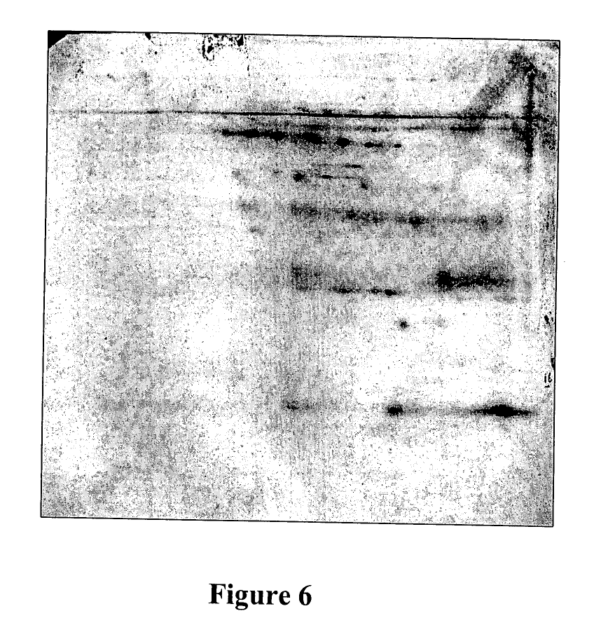

After the colloidal gold stain, the blots, films and 2-D gels were matched with each other (FIGS. 4, 5 and 6). This method allowed us to see which NPI Apo E spots (originally from the 2-D gels) could be detected by the antibody. The anti-Apo E antibody 13F4B5 was able to detect full size Apo E (±35 kDa) as well as some of the low molecular weight (LMW) Apo E forms (12-15 kDa) as described in the present invention. Immunodetection reveals that this antibody is able to detect the following LMW Apo E forms on a 2-D blot: NPI 60, NPI 75, NPI 73, and NPI 74.

Other antibodies (Table 7) are also tested for the detection of full size Apo E (±35 kDa) as well as low molecular weight (LMW) Apo E forms (12-15 kDa). The specificity of these antibodies for certain Apo E NPIs is being confirmed.

TABLES

TABLE 1
Characteristics of the CSF samples used in the 2-D analysis.
CSF samples were clinically diagnosed as AD (AD1-AD12),
FTD (FTD1-FTD6, B3, B4, B7 and B8), controls
(C1-C6), VAD (B1, B2, B5 and B6) and depression (D1-D6).

| Patient | | | | | | Aβ42 | |
|---|---|---|---|---|---|---|---|
| code | Diagnosis | Sex | Age | MMSE | Tau (pg/ml) | (pg/ml) | Albumin ratio |
| AD1 | AD | f | 78 | 22 | 545 | 299 | 3.63 |
| AD2 | AD | m | 72 | 18 | 1720 | 380 | 3.84 |
| AD3 | AD | f | 78 | 26 | 764 | 448 | 3.6 |
| AD4 | AD | m | 74 | 21 | 728 | 460 | 5.32 |
| AD5 | AD | f | 76 | 26 | 508 | 398 | 4.21 |
| AD6 | AD | m | 74 | 25 | 1220 | 418 | 5.5 |
| AD7 | AD | f | 79 | 19 | 710 | 288 | 4.8 |
| AD8 | AD | f | 88 | 18 | 592 | 440 | 6.6 |
| AD9 | AD | f | 75 | 21 | 516 | 499 | 4.6 |
| AD10 | AD | f | 89 | 17 | 741 | 390 | 6.9 |
| AD11 | AD | m | 88 | 18 | 636 | 324 | 6.4 |
| AD12 | AD | m | 76 | 18 | 599 | 395 | 3.3 |
| C1 | Control | m | 64 | 30 | 251 | 1070 | 2.93 |
| C2 | Control | m | 67 | 25 | 342 | 1084 | 5.77 |
| C3 | Control | m | 53 | 30 | 204 | 1079 | 3.97 |
| C4 | Control | f | 62 | 30 | 266 | 1125 | 3.42 |
| C5 | Control | m | 60 | 30 | 298 | 1108 | 4.31 |
| C6 | Control | f | 81 | 27 | 224 | 636 | 5.83 |
| FTD1 | FTD | f | 69 | 18 | 444 | 828 | 4.50 |
| FTD2 | FTD | f | 66 | 18 | 401 | 642 | 6.90 |
| FTD3 | FTD | f | 76 | 22 | 363 | 833 | 5.90 |
| FTD4 | FTD | m | 69 | 18 | 471 | 218 | 5.80 |
| FTD5 | FTD | f | 62 | 15 | 214 | NA | 7.70 |
| FTD6 | FTD | m | 71 | 16 | 348 | 823 | 7.80 |
| B1 | VAD | m | 71 | 8 | 573 | 303 | 6.8 |
| B2 | VAD | f | 79 | NA | NA | NA | NA |
| B3 | FTD | f | 71 | 25 | 388 | 148 | 16 |
| B4 | FTD | f | 63 | 19 | 737 | 257 | 5.9 |
| B5 | VAD | m | 73 | 22 | 318 | 213 | 6 |
| B6 | VAD | m | 76 | 19 | 244 | 718 | 6.3 |
| B7 | FTD | m | 68 | 25 | 778 | 510 | 5.5 |
| B8 | FTD | m | 47 | 25 | 430 | 644 | 10.4 |
| D1 | Depression | m | 69 | 28 | 256 | 730 | 5.4 |
| D2 | Depression | f | 81 | 27 | 330 | 558 | 4.8 |
| D3 | Depression | f | 76 | 29 | 514 | NA | 10.6 |
| D4 | Depression | m | 79 | 27 | 208 | NA | 7 |
| D5 | Depression | f | 44 | 30 | 342 | 1089 | 6.1 |
| D6 | Depression | f | 75 | 25 | 357 | 900 | 3.93 |

NA = not available;
m = male;
f = female;
MMSE = Mini-Mental State Examination score.

TABLE 2
Significantly altered protein isoforms as identified by MS sequencing.

| | | | | | QN | Fold | QL | |
|---|---|---|---|---|---|---|---|---|
| NPI | protein | Mw (kDa) | pI | ID number | p-value | difference | p-value | Counts |
| | AD < C | | | | | | | |
| 1 | Alpha-1-antitrypsin | 15.1 | 5.30 | P01009 | / | / | 0.005 | C: 7/12 AD: 1/12 |
| 25 | Prostaglandin-H2 D-isomerase | 27.3 | 5.44 | P41222 | / | / | 0.01 | C: 6/12 AD: 0/12 |
| 16 | Apolipoprotein J | 35.2 | 5.35 | P10909 | 0.02 | 1.8 | / | / |
| | FTD < C | | | | | | | |
| 5 | Apolipoprotein A-I | 24.5 | 5.22 | P02647 | 0.005 | 2.7 | / | / |
| 6 | Apolipoprotein A-I | 24.0 | 5.14 | P02647 | / | / | 0.03 | C: 7/12 FTD: 1/12 |
| 12 | Apolipoprotein J | 29.1 | 4.98 | P10909 | / | / | 0.01 | C: 10/12 FTD: 3/12 |
| 17 | Gelsolin | 29.9 | 5.16 | P06396 | 0.04 | 1.9 | / | / |
| 24 | Prostaglandin-H2 D-isomerase | 24.5 | 5.34 | P41222 | / | / | 0.01 | C: 6/12 FTD: 0/12 |
| | FTD > C | | | | | | | |
| 18 | Haptoglobin-1/2 | 12.5 | 5.19 | P00737 | / | / | 0.01 | FTD: 9/12 C: 2/12 |
| | | | | P00738 | | | | |
| 4 | Antithrombin-III | 62 | 5.12 | P01008 | / | / | 0.03 | FTD: 7/12 C: 1/12 |
| 8 | Apolipoprotein A-IV | 43.4 | 5.08 | P06727 | 0.01 | 2 | / | / |
| 9 | Apolipoprotein A-IV | 43.4 | 5.14 | P06727 | 0.03 | 1.5 | / | / |
| 10 | Apolipoprotein A-IV | 42.7 | 5.08 | P06727 | 0.04 | 1.6 | / | / |
| 19 | Hemopexin | 77.1 | 5.37 | P02790 | / | / | 0.04 | FTD: 9/12 C: 3/12 |
| 20 | Hemopexin | 75.9 | 5.46 | P02790 | 0.04 | 2.2 | / | / |
| 22 | Ig alpha-1 chain C region | 66.6 | 5.09 | P01876 | / | / | 0.01 | FTD: 10/12 C: 3/12 |
| 23 | Kininogen | 65.6 | 5.06 | P01042 | 0.03 | 1.7 | / | / |
| 28m | Transthyretin | 13.8 | 5.24 | P02766 | 0.05 | 1.6 | / | / |
| 70 | Apolipoprotein A-I | 24.2 | 5.16 | P02647 | 0.04 | 2 | / | / |
| | AD > FTD | | | | | | | |
| 5 | Apolipoprotein A-I | 24.5 | 5.22 | P02647 | 0.03 | 2 | / | / |
| 6 | Apolipoprotein A-I | 24.0 | 5.14 | P02647 | / | / | 0.03 | AD: 7/12 FTD: 1/12 |
| 26 | Transthyretin | 12.7 | 5.22 | P02766 | 0.04 | 1.7 | / | / |
| | FTD > AD | | | | | | | |
| 25 | Prostaglandin-H2 D-isomerase | 27.3 | 5.44 | P41222 | / | / | 0.01 | FTD: 6/12 AD: 0/12 |
| 2 | Alpha-1 beta-glycoprotein | 72.5 | 5.32 | P04217 | / | / | 0.03 | FTD: 11/12 AD: 5/12 |
| 3 | Alpha-1 beta-glycoprotein | 42.1 | 5.13 | P04217 | / | / | 0.009 | FTD: 8/12 C: 1/12 |
| 69 | Apolipoprotein A-I | 24.3 | 5.08 | P02647 | 0.03 | 1.7 | / | / |
| 71 | Apolipoprotein A-I | 20.2 | 5.35 | P02647 | 0.01 | 1.4 | / | / |
| 7 | Apolipoprotein A-I | 24.2 | 5.27 | P02647 | 0.03 | 1.5 | / | / |
| 8 | Apolipoprotein A-IV | 43.4 | 5.08 | P06727 | 0.001 | 2 | / | / |
| 9 | Apolipoprotein A-IV | 43.4 | 5.14 | P06727 | 0.04 | 1.5 | / | / |
| 11 | Apolipoprotein E | 35.3 | 5.22 | P02649 | / | / | 0.04 | FTD: 9/12 AD: 3/12 |
| 13 | Apolipoprotein J | 35.3 | 5.00 | P10909 | / | / | 0.04 | FTD: 12/12 AD: 7/12 |
| 14 | Apolipoprotein J | 36.6 | 5.07 | P10909 | 0.009 | 2 | / | / |
| 15 | Apolipoprotein J | 36.3 | 5.18 | P10909 | 0.01 | 1.8 | / | / |
| 16 | Apolipoprotein J | 35.2 | 5.35 | P10909 | / | / | 0.04 | FTD: 12/12 AD: 7/12 |
| 21 | Ig alpha-1 chain C region | 66.7 | 5.31 | P01876 | / | / | 0.01 | FTD: 10/12 AD: 3/12 |
| 22 | Ig alpha-1 chain C region | 66.6 | 5.09 | P01876 | / | / | 0.0001 | FTD: 10/12 AD: 0/12 |
| 27 | Transthyretin | 20.5 | 5.28 | P02766 | 0.04 | 1.6 | / | / |
| 28m | Transthyretin | 13.8 | 5.24 | P02766 | 0.02 | 1.4 | / | / |
| 29 | Vitamin D-binding protein | 57.0 | 5.29 | P02774 | 0.04 | 2.3 | 0.005 | FTD: 12/12 AD: 5/12 |
| 30 | Vitamin D-binding protein | 32.5 | 5.06 | P02774 | 0.04 | 1.8 | 0.005 | FTD: 12/12 AD: 5/12 |
| | AD > VAD | | | | | | | |
| 5 | Apolipoprotein A-I | 24.5 | 5.22 | P02647 | 0.004 | 2.6 | / | / |
| | AD < VAD | | | | | | | |
| 7 | Apolipoprotein A-I | 24.2 | 5.27 | P02647 | 0.001 | 1.7 | / | / |
| 74 | Apolipoprotein E | 15.8 | 4.91 | P02649 | 0.045 | 2.8 | / | / |
| 76m | Apolipoprotein E | 13.8 | 5.24 | P02649 | 0.03 | 1.5 | / | / |
| | AD < D | | | | | | | |
| 31 | Alpha-1-beta-glycoprotein | 79.2 | 5.18 | P04217 | 0.04 | 7.7 | / | / |
| 23 | Kininogen | 65.6 | 5.06 | P01042 | 0.04 | 5.1 | / | / |
| 33 | Zn-alpha-2-glycoprotein | 42.1 | 4.88 | P25311 | 0.01 | 7.2 | / | / |
| 34 | Apolipoprotein E | 35.2 | 5.11 | P02649 | 0.002 | 62 | / | / |
| 35 | Apolipoprotein E | 34.3 | 5.32 | P02649 | 0.04 | 24 | / | / |
| 37 | Apolipoprotein A-I | 24.0 | 5.10 | P02647 | 0.04 | 6.7 | / | / |
| 69 | Apolipoprotein A-I | 24.0 | 5.10 | P02647 | 0.04 | 6.7 | / | / |
| 6 | Apolipoprotein A-I | 24.0 | 5.14 | P02647 | 0.02 | 10. | / | / |
| 41 | Apolipoprotein E | 15.3 | 5.07 | P02649 | 0.02 | 2.9 | / | / |
| 42 | Alpha-1-antitrypsin | 58.5 | 4.92 | P01009 | 0.009 | 9.7 | / | / |
| 43 | Alpha-1-antitrypsin | 57.3 | 5.05 | P01009 | 0.02 | 7.1 | / | / |
| 44 | Alpha-1-antitrypsin | 57.7 | 5.00 | P01009 | 0.03 | 6.7 | / | / |
| 48 | Alpha-1-beta-glycoprotein | 79.2 | 5.18 | P04217 | 0.01 | 11 | / | / |
| 52 | Apolipoprotein E | 33.8 | 5.30 | P02649 | 0.03 | 24. | / | / |
| 59 | Alpha-1-antitrypsin | 58.5 | 4.92 | P01009 | 0.02 | 9.3 | / | / |
| 60 | Apolipoprotein E | 15.6 | 5.38 | P02649 | 0.02 | 4.6 | / | / |
| 12 | Apolipoprotein J | 30.3 | 5.01 | P10909 | 0.02 | 2.6 | / | / |
| | AD > D | | | | | | | |
| 66 | Apolipoprotein E | 30 | 5.17 | P02649 | / | / | 0.04 | AD: 5/12 D: 0/12 |

Each protein spot has an apparent molecular mass value (kDa) and approximate pI value relative to the gel region in which it was found.
Quantitative data (QN) and/or qualitative data (QN) are listed as fold differences and/or counts between groups.
NPI = neurological disease-associated protein isoform or protein number;
NA = not applicable;
m = two proteins identified in one spot;
ID number = protein identification in the Swiss Prot data bank;
Counts = number of data points, per group, for each protein isoform;
AD = Alzheimer's disease;
C = controls;
FTD = frontotemporal dementia;
VAD = vascular dementia;
D = depression.

TABLE 3
Differential expression of Apo A-I isoforms in
AD patients versus contrast groups.

| Apo A-I | | MW | 6 AD vs. | 6 AD vs. | 6 AD vs. | 6 AD vs. |
|---|---|---|---|---|---|---|
| NPI | pI | kDa | 6 FTD | 10 FTD | 4 VAD | 6 D |
| 69* | 5.08 | 24.3 | ↓ | ↓ | = | ↓ |
| 6 | 5.14 | 24 | ↑QL | ↑QL | ↑QL | ↓ |
| 70 | 5.16 | 24.2 | = | = | = | = |
| 5 | 5.22 | 24.5 | ↑ | ↑ | ↑ | = |
| 7 | 5.27 | 24.2 | ↓ | ↓ | ↓ | = |
| 71 | 5.35 | 20.2 | ↓ | ↓ | = | nd |
| 37* | 5.10 | 24 | ↓ | ↓ | = | ↓ |

Only quantitative results are shown (with the exception of NPI 6, where only a qualitative difference was found when comparing AD-FTD and AD-VAD);
↓: downregulated in AD (p < 0.05);
↑: upregulated in AD (p < 0.05);
=: no significant difference; magnitude of effect is shown in FIG. 3a and 3b;
nd: not detected;
*NPI 69 and 37 are not always resolved as a single spot on 2-D gel.
QL = qualitative difference.

TABLE 4
Differential expression of Apo E isoforms in AD patients versus contrast groups.

| Apo E | | | 6 AD vs. | 6 AD vs. | 6 AD vs. | 6 AD vs. | Aa # | |
|---|---|---|---|---|---|---|---|---|
| NPI | pI | MW (kDa) | 6 D | 6 FTD | 10 FTD | 4 VAD | start-end | Peptide sequence # |
| 34 | 5.11 | 35.2 | ↓ | = | = | = | 270-278 | LQAEAFQAR |
| | | | | | | | 259-269 | AKLEEQAQQIR |
| | | | | | | | 166-175 | LLRDADDLQK |
| | | | | | | | 19-33 | KVEQAVETEPEPELR |
| 35 | 5.32 | 34.3 | ↓ | nd | nd | nd | 270-278 | LQAEAFQAR |
| | | | | | | | 259-269 | AKLEEQAQQIR |
| | | | | | | | 199-207 | LGPLVEQGR |
| | | | | | | | 301-317 (C-term.) | VQAAVGTSAAPVPSDNH |
| 72 | 5.07 | 15.8 | = | = | nd | nd | 199-207 | LGPLVEQGR |
| | | | | | | | 270-278 | LQAEAFQAR |
| | | | | | | | 259-269 | AKLEEQAQQIR |
| 73 | 5.11 | 15.8 | = | = | = | = | 270-278 | LQAEAFQAR |
| 74 | 4.91 | 15.8 | = | = | = | ↓ | 199-207 | LGPLVEQGR |
| | | | | | | | 270-278 | LQAEAFQAR |
| | | | | | | | 259-269 | AKLEEQAQQIR |
| | | | | | | | 210-224 | AATVGSLAGQPLQER |
| | | | | | | | 138-152 | GEVQAMLGQSTEELR |
| | | | | | | | 94-108 | SELEEQLTPVAEETR |
| | | | | | | | 301-317 (C-term.) | VQAAVGTSAAPVPSDNH |
| 75 | 5.09 | 15.1 | = | nd | nd | nd | 138-152 | GEVQAMLGQSTEELR |
| | | | | | | | 19-33 | KVEQAVETEPEPELR |
| | | | | | | | 270-278 | LQAEAFQAR |
| 41 | 5.07 | 15.3 | ↓ | nd | nd | nd | 19-33 | KVEQAVETEPEPELR |
| | | | | | | | 270-278 | LQAEAFQAR |
| 76 | 5.24 | 13.8 | =(↓) | ↓ | = | ↓ | 210-224 | AATVGSLAGQPLQER |
| 77 | 4.96 | 12.4 | =(↑) | = | = | = | 259-269 | AKLEEQAQQIR |
| 52 | 5.30 | 33.8 | ↓ | nd | nd | nd | 270-278 | LQAEAFQAR |
| | | | | | | | 199-207 | LGPLVEQGR |
| | | | | | | | 259-269 | AKLEEQAQQIR |
| | | | | | | | 80-90 | ALMDETMKELK |
| 60 | 5.38 | 15.6 | ↓ | nd | nd | nd | 19-33 | KVEQAVETEPEPELR |
| 66 | 5.17 | 30. | ↑QL | nd | nd | nd | 199-207 | LGPLVEQGR |
| | | | | | | | 91-109 | AYKSELEEQLTPVAEETR |
| | | | | | | | 111-121 | LSKELQAAQAR |
| | | | | | | | 210-224 | AATVGSLAGQPLQER |
| | | | | | | | 259-269 | AKLEEQAQQIR |
| | | | | | | | 270-278 | LQAEAFQAR |
| 11 | 5.22 | 35.3 | nd | ↓QL | nd | nd | 19-33 | KVEQAVETEPEPELR |
| | | | | | | | 91-109 | AYKSELEEQLTPVAEETR |
| | | | | | | | 111-121 | LSKELQAAQAR |
| | | | | | | | 210-224 | AATVGSLAGQPLQER |
| | | | | | | | 259-269 | AKLEEQAQQIR |
| | | | | | | | 270-278 | LQAEAFQAR |

Only quantitative results are showed (with the exception of NPI 66 and NPI 11, where only a qualitative difference (QL) was found).
↓: down-regulated in AD {↓: p < 0.05; =(↓): p < 0.07}
↑: upregulated in AD {↑: p < 0.05; =(↑): p < 0.07}
=: no significant difference
#: peptide sequences covered by MS analysis
nd: not detected

TABLE 5
Identification of the protein spots that were altered between the studied groups.

| Spot | Spot | | | | ID number |
|---|---|---|---|---|---|
| exp1 | exp2 | Peptide | aa | Identification in database | database |
| 2713 | 674 | TDTSHHDQDHPTFNK | 35-49 | Alpha-1-antitrypsin | P01009 |
| | | LVDKFLEDVK | 150-159 | | |
| | | FLEDVKK | 154-160 | | |
| | | KQINDYVEK | 179-187 | | |
| | | QINDYVEK | 180-187 | | |
| | | DTEEEDFHVDQATTVK (M1A allele) | 226-241 | | |
| | | DTEEEDFHVDQVTTVK (M1V allele) | 226-241 | | |
| | | LQHLENELTHDIITK | 284-298 | | |
| | | FLENEDR | 299-305 | | |
| | | FLENEDRR | 299-306 | | |
| | | SASLHLPK | 307-314 | | |
| | | LSITGTYDLK | 315-324 | | |
| | | SVLGQLGITK | 325-334 | | |
| | | VFSNGADLSGVTEEAPLK | 335-352 | | |
| | | AVLTIDEK | 360-367 | | |
| 4704 | 353 | TDTSHHDQDHPTFNK | 35-49 | Alpha-1-antitrypsin | P01009 |
| | | FLEDVKK | 154-160 | | |
| | | KQINDYVEK | 179-187 | | |
| | | QINDYVEK | 180-187 | | |
| | | DTEEEDFHVDQATTVK (M1A allele) | 226-241 | | |
| | | DTEEEDFHVDQVTTVK (M1V allele) | 226-241 | | |
| | | LSSWVLLMK + 1 Oxidation (M) | 259-267 | | |
| | | FLENEDR | 299-305 | | |
| | | FLENEDRR | 299-306 | | |
| | | SASLHLPK | 307-314 | | |
| | | LSITGTYDLK | 315-324 | | |
| | | SVLGQLGITK | 325-334 | | |
| | | VFSNGADLSGVTEEAPLK | 335-352 | | |
| | | AVLTIDEK | 360-367 | | |
| | | LGMFNIQHCK (Cys-CAM) | 248-257 | | |
| 4705 | 355 | QINDYVEK | 180-187 | Alpha-1-antitrypsin | P01009 |
| 7206 | | LSITGTYDLK | 315-324 | Alpha-1-antitrypsin | P01009 |
| | | SVLGQLGITK | 325-334 | | |
| | | VFSNGADLSGVTEEAPLK | 335-352 | | |
| 4801RBH | | LLELTGPK | 86-93 | Alpha-1B-glycoprotein | P04217 |
| 4803 | 375 | ATWSGAVLAGR | 386-396 | Alpha-1B-glycoprotein | P04217 |
| | | FALVREDR | 313-320 | | |
| 1RBH | | CLAPLEGAR (cys-CAM + ox) | 304-312 | Alpha-1B-glycoprotein | P04217 |
| | | FALVREDR | 313-320 | | |
| 901RBH | | LETPDFQLFK | 32-41 | Alpha-1B-glycoprotein | P04217 |
| | | ATWSGAVLAGR | 386-396 | | |
| | | LLELTGPK | 86-93 | | |
| 5702 | | EVPLNTIIFMGR + 1 Oxidation (M) | 446-457 | Antithrombin-III | P01008 |
| 6102 | 149 | DYVSQFEGSALGK | 52-64 | Apolipoprotein A-I | P02647 |
| | | VQPYLDDFQK | 121-130 | | |
| | | [916.56]⁺ ALKED[360.25]⁺ (aa 208: N → D) | 201-212 | | |
| | | ATEHLSTLSEK | 220-230 | | |
| | | AKPALEDLR | 231-239 | | |
| | | LSPLGEEMR + 1 Oxidation (M) | 165-173 | | |
| | | THLAPYSDELR | 185-195 | | |
| | | LLDNWDSVTSTFSK | 70-83 | | |
| | | DSGRDYVSQFEGSALGK | 48-64 | | |
| | | LEALKENGGAR | 202-212 | | |
| | | QGLLPVLESFK | 240-250 | | |
| | | VEPLRAELQEGAR | 143-155 | | |
| 6303 | 146 | DYVSQFEGSALGK | 52-64 | Apolipoprotein A-I | P02647 |
| | | VQPYLDDFQK | 121-130 | | |
| | | [916.56]⁺ALKED[360.27]⁺ (aa208: N→D) | 201-212 | | |
| | | ATEHLSTLSEK | 220-230 | | |
| | | LSPLGEEMRDR + 1 Oxidation (M) | 165-175 | | |
| | | LSPLGEEMR + 1 Oxidation (M) | 165-173 | | |
| | | THLAPYSDELR | 185-195 | | |
| 7101 | 285 | DSGRDYVSQFEGSALGK | 48-64 | Apolipoprotein A-I | P02647 |
| | | DYVSQFEGSALGK | 52-64 | | |
| | | [1315.83]⁺DNDDSVTSTFSK (aa 74: W→D) | 70-83 | | |
| | | QEMSKDLEEVK + 1 Oxidation (M) | 108-118 | | |
| | | VQPYLDDFQK | 121-130 | | |
| | | VQPYLDDFQKK | 121-131 | | |
| | | LSPLGEEMR + 1 Oxidation (M) | 165-173 | | |
| | | LSPLGEEMRDR + 1 Oxidation (M) | 165-175 | | |
| | | THLAPYSDELR | 185-195 | | |
| | | LEALKED[360.25]⁺ (aa 208: N→D) | 202-212 | | |
| | | LEALKENGGAR | 202-212 | | |
| | | ATEHLSTLSEK | 220-230 | | |
| | | AKPALEDLR | 231-239 | | |
| | | DLATVYVDVLK | 237-247 | | |
| | | QGLLPVLESFK | 240-250 | | |
| | | QKLHELQEK (E → pyroglutamic acid) | 156-164 | | |
| | | VEPLRAELQEGAR | 143-155 | | |
| | | LLDNWDSVTSTFSK | 70-83 | | |
| | | DEPPQSPWDR + 1 Oxidation (W) | 25-34 | | |
| | | DLATVYVDVLK | 37-47 | | |
| | | VSFLSALEEYTK | 251-262 | | |
| | | KWQEEMELYR + 1 Oxydation (M) | 131-140 | | |
| 4310 | 144 | THLAPYSDELR | 185-195 | Apolipoprotein A-I | P02647 |
| | 143 | THLAPYSDELR | 185-195 | Apolipoprotein A-I | P02647 |
| 4606 + 4605 | | LGEVNTYAGDLQK | 66-78 | Apolipoprotein A-IV | P06727 |
| | | LLPHANEVSQK | 113-123 | | |
| | | QLTPYAQR | 156-163 | | |
| | | IDQNVEELKGR | 190-200 | | |
| | | LTPYADEFK | 201-209 | | |
| | | ISASAEELR | 256-264 | | |
| | | LAPLAEDVR | 267-275 | | |
| | | ALVQQMEQLR + 1 Oxidation (M) | 317-326 | | |
| 5402 | 81 | LEPYADQLR | 135-143 | Apolipoprotein A-IV | P06727 |
| | | IDQNVEELKGR | 190-200 | | |
| | | LTPYADEFK | 201-209 | | |
| | | IDQTVEELR | 212-220 | | |
| | | ISASAEELR | 257-264 | | |
| | | LAPLAEDVR | 267-275 | | |
| | | ALVQQMEQLR + 1 Oxidation (M) | 317-326 | | |
| | | RVEPYGENFNK | 306-317 | | |
| | | SLAPYAQDTQEK | 222-233 | | |
| | | LGEVNTYAGDLQK | 66-78 | | |
| 6502 | | KVEQAVETEPEPELR | 19-33 | Apolipoprotein E | P02649 |
| | | AYKSELEEQLTPVAEETR | 91-109 | | |
| | | LSKELQAAQAR | 111-121 | | |
| | | AATVGSLAGQPLQER | 210-224 | | |
| | | AKLEEQAQQIR | 259-269 | | |
| | | LQAEAFQAR | 270-278 | | |
| 5502 | 110 | LQAEAFQAR | 270-278 | Apolipoprotein E | P02649 |
| | | AKLEEQAQQIR | 259-269 | | |
| | | LLRDADDLQK | 166-175 | | |
| | | KVEQAVETEPEPELR | 19-33 | | |
| | 114 | LQAEAFQAR | 270-278 | Apolipoprotein E | P02649 |
| | | AKLEEQAQQIR | 259-269 | | |
| | | LGPLVEQGR | 199-207 | | |
| | | VQAAVGTSAAPVPSDNH | 301-317 | | |
| | 272 | LGPLVEQGR | 199-207 | Apolipoprotein E | P02649 |
| | 862 | LGPLVEQGR | 199-207 | Apolipoprotein E | P02649 |
| | 681 | KVEQAVETEPEPELR | 19-33 | Apolipoprotein E | P02649 |
| | 480 | LQAEAFQAR | 270-278 | Apolipoprotein E | P02649 |
| | | LGPLVEQGR | 199-207 | | |
| | | AKLEEQAQQIR | 259-269 | | |
| | | ALMDETMKELK + 2 Oxidations (M) | 80-90 | | |
| 3405 | | ELDESLQVAER | 326-336 | Apolipoprotein J | P10909 |
| 3505 | | ELDESLQVAER | 326-336 | Apolipoprotein J | P10909 |
| 4401 | 323 | ELDESLQVAER | 326-336 | Apolipoprotein J | P10909 |
| | | KYNELLK | 340-346 | | |
| | | FMETVAEK + 1 Oxidation (M) | 430-437 | | |
| 5302 | 108 | ELDESLQVAER | 326-336 | Apolipoprotein J | P10909 |
| | | EILSVDCSTNNPSQAK + 1 (cys-CAM) | 307-322 | | |
| 8601 | | TLLSNLEEAK | 69-78 | Apolipoprotein J | P10909 |
| | | IDSLLENDR | 159-167 | | |
| | | ASSIIDELFQDR | 183-194 | | |
| 5202 | | AGALNSNDAFVLK | 585-597 | Gelsolin | P06396 |
| | | YIETDPANR | 730-738 | | |
| 6404 | | AGALNSNDAFVLK | 585-597 | Gelsolin | P06396 |
| | | TGAQELLR | 616-623 | | |
| 5004 | 411 | TEGDGVYTLNDKK | 60-72 | Haptoglobin-1/2 | P00737 |
| | | | 119-131 | P00738 | |
| | | TEGDGVYTLNDKKQWINK + 1 ox (W) | 60-77 | | P00737 |
| | | | 119-136 | | P00738 |
| 5903RBH | | NFPSPVDAAFR | 92-102 | Hemopexin | P02790 |
| | | GGYTLVSGYPK | 333-343 | | |
| 8902RBH | | NFPSPVDAAFR | 92-102 | Hemopexin | P02790 |
| | | QGHNSVFLIK | 103-112 | | |
| | | DYFMPCPGR + 1 (cys-CAM + ox) + 1 ox (M) | 226-234 | | |
| | | GGYTLVSGYPK | 333-343 | | |
| 4701RBH | | SAVQGPPER | 169-177 | Ig alpha-1 chain C region | P01876 |
| | | | | (heavy) | |
| | | QEPSQGTTTFAVTSILR | 283-299 | | |
| 4804 | | TPLTATLSK | 213-221 | Ig alpha-1 chain C region | P01876 |
| | | | | (heavy) | |
| 4702 | | TVGSDTFYSFK | 65-75 | Kininogen | P01042 |
| | | QVVAGLNFR | 188-196 | | |
| | | YFIDFVAR | 317-324 | | |
| 8101 | | APEAQVSVQPNFQQDK | 23-38 | Prostaglandin-H2 D- | P41222 |
| | | | | isomerase | |
| | | TMLLQPAGSLGSYSYR + 1 Oxidation (M) | 93-108 | | |
| | | AQGFTEDTIVFLPQTDK | 169-185 | | |
| 9209 | | [1617.85]⁺ EAQVSVQPNF[518.26]⁺ | 23-38 | Prostaglandin-H2 D- | P41222 |
| | | | | isomerase | |
| | | TMLLQPAGSLGSYSYR + 1 Oxidation (M) | 93-108 | | |
| 6001 | | AADDTDEPFASGK (aa 61: W→D) | 56-68 | Transthyretin | P02766 |
| 7102 | | [603.41]PLMVK | 21-35 | Transthyretin | P02766 |
| 7108 | 274 | GPTGTGESKCPLMVK (Cys(O₃H)) | 21-35 | Transthyretin | P02766 |
| | | GPTGTGESKCPLMVK (Cys(O₃H)/M: oxidation to | 21-35 | | |
| | | sulphone) | | | |
| | | AADDTWEPFASGK (W + 2*16 Da) | 56-68 | | |
| | | AADDTDEPFASGK (aa 61: W→D) | 56-68 | | |
| | | AADDTWEPFASGK | 56-68 | | |
| | | KAADDTWEPFASGK | 55-68 | | |
| | | TSESGELHGLTTEEEFVEGIYK | 69-90 | | |
| 3601RBH | | HLSLLTTLSNR | 208-218 | Vitamin D-binding protein | P02774 |
| | | YTFELSR | 346-352 | | |
| | | THLPEVFLSK | 354-363 | | |
| | | VLEPTLK | 364-370 | | |
| | | ELSSFIDK | 395-402 | | |
| 4411 | | VCSQYAAYGEK (cys-CAM + ox) | 219-229 | Vitamin D-binding protein | P02774 |
| | | VMDKYTFELSR + 1 Oxidation (M) | 342-352 | | |
| | | YTFELSR | 346-352 | | |
| | | THLPEVFLSK | 354-363 | | |
| | | VLEPTLK | 364-370 | | |
| | | [1433.61]⁺CCDVEDSTTCFNAK (1 cys-CAM + ox, 2 | 371-388 | | |
| | | Dha) | | | |
| | | ELSSFIDK | 395-402 | | |
| | | AKLPDATPK | 428-436 | | |
| 2402 | 88 | AGEVQEPELR | 239-248 | Zinc-alpha-2-glycoprotein | P25311 |
| | | QDPPSVVVTSHQAPGEK | 201-217 | | |

TABLE 6
Protein isoforms, identified on a 2D-gel, that are significantly altered in CSF
obtained from patients suffering from AD compared to CSF obtained from patients
suffering from depression (D). Each protein spot has an apparent molecular mass
value and an approximate pI value respective to the gel region in which it was
identified.

| | | | | p-value | | | | |
|---|---|---|---|---|---|---|---|---|
| NPI | Spot no. | MW (kDa) | pI | QN | QL | D/AD | AD/D | Comparison |
| 32 | 61 | 57.4 | 5.08 | 0.0341 | ns | 6.41 | 0.16 | AD < D |
| 36 | 120 | 32.4 | 4.9 | / | 0.0373 | / | / | AD < D |
| 38 | 173 | 17.8 | 5.13 | / | 0.0137 | / | / | AD < D |
| 39 | 178 | 16.7 | 5.01 | 0.0032 | ns | 0.25 | 4.02 | AD > D |
| 40 | 184 | 15.6 | 5.24 | 0.0329 | ns | 7.36 | 0.14 | AD < D |
| 45 | 366 | 63.1 | 5.12 | / | 0.0272 | 1.07 | 0.94 | AD < D |
| 46 | 370 | 53.1 | 5.23 | / | 0.0361 | 1.14 | 0.88 | AD < D |
| 47 | 377 | 82.1 | 5.03 | 0.0273 | ns | 5.72 | 0.17 | AD < D |
| 49 | 409 | 15.1 | 5.17 | / | 0.0001 | / | / | AD > D |
| 50 | 465 | 13.8 | 5.37 | 0.0481 | ns | 0.28 | 3.57 | AD > D |
| 51 | 470 | 50.7 | 5.13 | 0.0212 | ns | 3.62 | 0.28 | AD < D |
| 53 | 482 | 56.5 | 5.15 | / | 0.0373 | / | / | AD < D |
| 54 | 486 | 15.6 | 5.32 | / | 0.0391 | 1.14 | 0.88 | AD < D |
| 55 | 513 | 13.7 | 5.4 | / | 0.0272 | 2.40 | 0.42 | AD < D |
| 56 | 599 | 82.7 | 4.99 | 0.0394 | ns | 11.08 | 0.09 | AD < D |
| 57 | 639 | 57.7 | 4.52 | / | 0.0137 | / | / | AD > D |
| 58 | 646 | 55.5 | 5.12 | 0.0013 | ns | 11.90 | 0.08 | AD < D |
| 61 | 699 | 31.4 | 5.09 | / | 0.0373 | / | / | AD < D |
| 62 | 729 | 17 | 5.4 | 0.0259 | ns | 0.23 | 4.39 | AD > D |
| 63 | 798 | 35.4 | 5.14 | 0.0236 | ns | 4.48 | 0.22 | AD < D |
| 64 | 813 | 30.2 | 5.38 | 0.0321 | ns | 0.36 | 2.81 | AD > D |
| 65 | 839 | 13.8 | 4.64 | 0.0332 | ns | 0.50 | 1.98 | AD > D |
| 67 | 968 | 33.1 | 5.37 | / | 0.0361 | 0.56 | 1.79 | AD > D |
| 68 | 1004 | 15.7 | 5.37 | / | 0.0373 | / | / | AD < D |

ns: no statistically significant difference was found;
/: not applicable.
QN: quantitative difference;
QL: qualitative difference.

TABLE 7
Overview of some antibodies that are available for detection of Apo E isoforms.

| | Company, Code | Isotype | Epitope | Western-blot results |
|---|---|---|---|---|
| 13F4B5 | Innogenetics N.V, Ghent, Belgium, | IgG1κ | Not known | Full size Apo E; |
| | M-012 (90327, IHG-29) | | | LMW Apo E: NPI 60, NPI 75, NPI 73, NPI 74 |
| 9H10C6 | Innogenetics N.V., Ghent, Belgium, | Not known | Not known | Full size Apo E; |
| | IGH-28 | | | LMW Apo E: to be confirmed |
| A0077 | Dako, Glostrup, Denmark | Rb poly | Not known | Full size Apo E; |
| | | | | LMW Apo E: to be confirmed |
| A1.4 | Santa Cruz, Biotecnology, Inc., Santa | IgG1 | 126-191 | Full size Apo E; |
| | Cruz, CA, US, Sc-13521 | | | LMW Apo E: to be confirmed |
| D6E10 | Signet Laboratories, Inc., Dedham, | IgG1 | 158 | Full size Apo E; |
| | MA, US | | | LMW Apo E: to be confirmed |
| 3D12 | Medical and Biological Laboratories | IgG2a | Not known | Full size Apo E; |
| | Co., Lnaka-ku Nagoya, Japan | | | LMW Apo E: to be confirmed |
| 1F9 | Medical and Biological Laboratories | IgG1 | 109-119, E4 specific | Full size Apo E; |
| | Co., Lnaka-ku Nagoya, Japan | | | LMW Apo E: to be confirmed |

### References

Alagiakrishnan K., Masaki K. (2001) Vascular Dementia. eMedicine Journal Vol. 2, Number 10.

Andreasen N., Minthon L., Clarberg A., Davidsson P., Gottfries J., Vanmechelen E., Vanderstichele H., Winblad B., Blennow K. (1999) Sensitivity, specificity, and stability of CSF-tau in AD in a community-based patient sample. Neurology 22: 1488-1494.

Andreasen N., Minthon L., Davidsson P., Vanmechelen E., Vanderstichele H., Winblad B., Blennow K. (2001) Evaluation of CSF-tau and CSF-Abeta42 as diagnostic markers for Alzheimer disease in clinical practice. Arch. Neurol. 58: 373-379.

Arbit E., Cheung N. K., Yeh S. D., Daghighian F., Shang J. J., Cordon-Cardo C., Pentlow K., Canete A., Finn R., Larson S. M. (1995) Quantitative studies of monoclonal antibody targeting to disialogangliosid GD2 in human brain tumors. Eur. J. Nucl. Med. 22: 419-426.

Aronson S. C. (2002) Depression. eMedicine Journal, Vol. 3, Number 1.

Blennow K., Hesse C., Fredman P. (1994) Cerebrospinal fluid apolipoprotein E is reduced in Alzheimer's disease. Neuroreport5: 2534-2536.

Celis J. E., Gromov P., Ostergaard M., Madsen P., Honore B., Dejgaard K., Olsen E., Vorum H., Kristensen D. B., Gromova I., Haunso A., Van Damme J., Puype M., Vandekerckhove J., Rasmussen H. H. (1996) Human 2-D PAGE databases for proteome analysis in health and disease: http://biobase.dk/cgi-bin/celis. FEBS Lett. 398: 129-134.

Cole et al. (1985) In: Monoclonal Antibodies and Cancer Therapy. Alan R. Liss, Inc. pp. 77-96.

Csemansky J. G., Miller J. P., McKeel D., Morris J. C. (2002) Relationships among cerebrospinal fluid biomarkers in dementia of the Alzheimer type. Alzheimer Dis. Assoc. Disord. 16: 144-149.

Davidsson P., Westman A., Puchades M., Nilsson C. L., Blennow K. (1999) Characterization of Proteins from Human Cerebrospinal Fluid by a Combination of Preparative Two-Dimensional Liquid-Phase Electrophoresis and Matrix-Assisted Laser Desorption/Ionization Time-of-Flight Mass Spectrometry. Anal. Chem. 71: 642-647.

Davidsson P., Westman-Brinkmalm A., Nilsson C. L., Lindbjer M., Paulson L., Andreasen N., Sjogren M., Blennow K. (2002) Proteome analysis of cerebrospinal fluid proteins in Alzheimer patients. Neuroreport 13: 611-615.

Demeester N., Castro G., Desrumaux C., De Geitere C., Fruchart J. C., Santens P., Mulleners E., Engelborghs S., De Deyn P. P., Vandekerckhove J., Rosseneu M., Labeur C. (2000) Characterization and functional studies of lipoproteins, lipid transfer proteins, and lecithin: cholesterol acyltransferase in CSF of normal individuals and patients with Alzheimer's disease. J. Lipid Res. 41: 963-974.

Fukuyama R., Mizuno T., Mori S., Yanagisawa K., Nakajima K., Fushiki S. (2000) Age-dependent decline in the apolipoprotein E level in cerebrospinal fluid from control subjects and its increase in cerebrospinal fluid from patients with Alzheimer's disease. Eur. Neurol43: 161-169.

Ghindilis A. L., Pavlov A. R., Atanassov P. B. (eds.) (2002) Immunoassay Methods and Protocols. Humana Press, Totowa, N.J., US.

Hesse C., Larsson H., Fredman P., Minthon L., Andreasen N., Davidsson P., Blennow K. (2000) Measurement of apolipoprotein E (apoE) in cerebrospinal fluid. Neurochem. Res.25: 511-517.

Hsich G., Kenney K., Gibbs C. J., Lee K. H., Harrington M. G. (1996) The 14-3-3 brain protein in cerebrospinal fluid as a marker for transmissible spongiform encephalopathies. N. Engl. J. Med. 335: 924-930.

Huang Q., He G., Lan Q., Li X., Qian Z. Chen J. Lu Z., Du Z. (1996) Target imaging diagnosis of human brain glioma. Clinical analysis of 40 cases. Nucl. Med. Commun. 17:311-316.

IFCC. (1987) Approved recommendation on the theory of reference values. Part. 5. Statistical treatment of collected refereece values. Determination of reference limits. J. Clin. Chem. Clin. Biochem. 25: 645-656.

Johnson G., Brane D., Block W., van Kammen D. P., Gurklis J., Peters J. L., Wyatt R. J., Kirch D. G., Ghanbari H. A., Merril C. R. (1992) Cerebrospinal fluid protein variations in common to Alzheimer's disease and schizophrenia. Appl. Theor. Electrophor.3: 47-53.

Klose J., Kobalz U. (1995) Two-dimensional electrophoresis of proteins: an updated protocol and implications for a functional analysis of the genome. Electrophoresis 16: 1034-1059.

Knopman D., Ritchie K., Polge C., Alafuzoff I., Soininen H. (2002) Alzheimer's disease. In: Qizilbash N. (ed.) Evidence-based Dementia Practice. Oxford: Blackwell Science, p. 228-259.

Kohler G., Milstein C. (1975) Continuous cultures of fused cells secreting antibody of predefined specificity. Nature 256: 495-497.

Kozbor D., Dexter D., Roder J. C. (1983) A comparative analysis of the phenotypic characteristics of available fusion partners for the construction of human hybridomas. Hybridoma 2:7-16.

Landen M., Hesse C., Fredman P., Regland B., Wallin A., Blennow K. (1996) Apolipoprotein E in cerebrospinal fluid from patients with Alzheimer's disease and other forms of dementia is reduced but without any correlation to the apoE4 isoform. Dementia.7: 273-278.

Leys D., Englund E., Erkinjuntii T. (2002) Vascular dementia. In: Qizilbash N. (ed.) Evidence-based Dementia Practice. Oxford: Blackwell Science, p. 260-287.

Lowe J. (2001) The pathological diagnosis of neurodegenerative diseases causing dementia. Curr. Top. Pathol.95: 149-177.

Lütjohann D., Papassotiropoulos A., Björkhem I., Locatelli S., Bagli M., Oehring R. D., Schlegel U., Jessen F., Rao M. L., von Bergmann K., Heun R. (2000) Plasma 24S-hydroxycholesterol (cerebrosterol) is increased in Alzheimer and vascular demented patients. J. Lipid Res. 41: 195-198.

Mariani G., Lasku A., Pau A., Villa G., Motta C., Calcagno G., Taddei G. Z., Castellani P., Syrigos K., Dorcaratto A., Epenetos A. A., Zardi L., Viale G. A. (1997) A pilot pharmacokinetic and immunoscintigraphic study with the technetium-99m labeled monoclonal antibody BC-1 directed against oncofetal fibronectin in patients with brain tumours. Cancer 15: 2484-2489.

Marin D. B., Sewell M. C., Schlechter A. (2002) Alzheimer's disease: Accurate and early diagnosis in the primary care setting. Geriatrics 57: 36-40.

McKeith I. G., Galasko D., Kosaka K., Perry E. K., Dickson D. W., Hansen L. A., Salmon D. P., Lowe J., Mirra S. S., Byrne E. J., Lennox G., Quinn N. P., Edwardson J. A., Ince P. G., Bergeron C., Bums A., Miller B. L., Lovestone S., Collerton D., Jansen E. N., Ballard C., de Vos R. A., Wilcock G. K., Jellinger K. A., Perry, R. H. (1996) Consensus guidelines for the clinical and pathologic diagnosis of dementia with Lewy bodies (DLB): report of the consortium on DLB international workshop. Neurology 47: 1113-1124.

McKeith I. G., O'Brien J. T., Ballard C. (1999) Diagnosing dementia with Lewy bodies. Lancet 354: 1227-1228.

McKeith I. G. (2002) Dementia with Lewy bodies. Br. J. Psychiatry 180: 144-147.

McKhann G., Drachman D., Folstein M., Katzman R., Price D., Stadlan E. M. (1984) Clinical diagnosis of Alzheimer's disease: report of the NINCDS-ADRDA Work Group under the auspices of Department of Health and Human Services Task Force on Alzheimer's Disease. Neurology 1984 34: 939-944.

Moghul S., Wilkinson D. (2001) Use of acetylcholinesterase inhibitors in Alzheimer's disease. Expert Rev. in Neurotherapeutics 1: 61.

Molina L., Touchon J., Herpe M., Lefranc D., Duplan L., Cristol J. P., Sabatier R., Vermersch P., Pau B., Mourton-Gilles C. (1999) Tau and apo E in CSF: potential aid for discriminating Alzheimer's disease from other dementias. Neuroreport 10: 3491-3495.

Monroe et al. (1986) Amer. Clin. Prod. Rev. 5: 34-41

Montine T. J., Markesbery W. R., Morrow J. D., Robers L. J. 2^{nd} (1998) Cerebrospinal fluid F2-isoprostane levels are increased in Alzheimer's disease. Ann. Neurol. 44: 410-413.

Montine T. J., Sidell K. R., Crews B. C., Markesbery W. R., Marnett L. J., Robers L. J. 2^{nd}, Morrow J. D. (2000) Elevated CSF prostaglandin E2 levels in patients with probable AD. Neurology 53: 1495-1498.

O' Farrell P. H. (1975) High resolution two-dimensional electrophoresis of proteins. J Biol. Chem. 250: 4007-4021.

Patterson S. D., Aebersold R. (1995) Mass spectrometric approaches for the identification of gel-separated proteins. Electrophoresis 16: 1791-814.

Petrella J. R., Coleman R. E., Doraiswamy P. M. (2003) Neuroimaging and early diagnosis of Alzheimer disease: a look to the future. Radiology 226: 315-336.

Poduslo J. F., Wengenack T. M., Curran G. L., Wisniewski T., Sigurdsson E. M., Macura S. I., Borowski B. J., Jack C. R. Jr. (2002) Molecular trageting of Alzheimer's amyloid plaques for contrast-enhanced magnetic resonance imaging. Neurobiol. Dis. 11: 315-329.

Qiu C., Skoog I., Fratiglioni L. (2002) Occurrence and determinants of vascular cognitive impairment. In: Erkinjuntti T., Gauthier S. (eds). Vascular Cognitive Impairment. London: Martin Dunitz pp 61-83.

Reiber H. (2001) Dynamics of brain-derived proteins in cerebrospinal fluid. Clin. Chim. Acta 310: 173-186.

Roman G. C., Tatemichi T. K., Erkinjuntti T., Cummings J. L., Masdeu J. C., Garcia J. H., Amaducci L., Orgogozo J. M., Brun A., Hofinan A., et al. (1993) Vascular dementia: diagnostic criteria for research studies. Report of the NINDS-AIREN International Workshop. Neurology 43: 250-260.

Rosler N., Wichart I., Jellinger K. A. (2001) Clinical significance of neurobiochemical profiles in the lumbar cerebrospinal fluid of Alzheimer's disease patients. J. Neural. Transm. 108: 231-246.

Sandrock D., Verheggen R., Helwig A. T., Munz D. L., Markakis E., Emrich D. (1996) Immunoscintigraphy for the detection of brain abscesses. Nucl. Med. Commun. 17: 311-316.

Siest G., Bertrand P., Qin B., Herbeth B., Serot J. M., Masana L., Ribalta J., Passmore A. P., Evans A., Ferrari M., Franceschi M., Shepherd J., Cuchel M., Beisiegel U., Zuchowsky K., Rukavina A. S., Sertic J., Stojanov M., Kostic V., Mitrevski A., Petrova V., Sass C., Merched A., Salonen J. T., Tiret L., Visvikis S. (2000) Apolipoprotein E polymorphism and serum concentration in Alzheimer's disease in nine European centres: the ApoEurope study. ApoEurope group. Clin. Chem. Lab. Med. 38: 721-730.

Sjogren M., Blomberg M., Jonsson M., Wahlund L. O., Edman A., Lind K., Rosengren L., Blennow K., Wallin A. (2001) Neurofilament protein in cerebrospinal fluid: a marker of white matter changes. J. Neurosci. Res. 66: 510-516.

Skoog I., Hesse C., Fredman P., Andreasson L. A., Palmertz B., Blennow K. (1997) Apolipoprotein E in cerebrospinal fluid in 85-year-old subjects. Relation to dementia, apolipoprotein E polymorphism, cerebral atrophy, and white matter lesions. Arch. Neurol. 54: 267-272.

Slooter A. J., de Knijff P., Hofman A., Cruts M., Breteler M. M., Van Broeckhoven C., Havekes L. M., van Duijn C. M. (1998) Serum apolipoprotein E level is not increased in Alzheimer's disease: the Rotterdam study. Neurosci. Lett. . 248: 21-24.

Small G. W. (2002) Brain-imaging surrogate markers for detection and prevention of age-related memory loss. Review. J. Mol. Neurosci. 19: 17-21.

Snowden J. S., Heary D., Mann D. M. A. (2002) Frontotemporal dementia. Br. J. Psychiatry180: 140-143.

Sunderland T., Wolozin B., Galasko D., Levy J., Dukoff R., Bahro M., Lasser R., Motter R., Lehtimaki T., Seubert P. (1999) Longitudinal stability of CSF tau levels in Alzheimer patients. Biol. Psychiatry 46:750-755.

Taddei K., Clamette R., Gandy S. E., Martins R. N. (1997) Increased plasma apolipoprotein E (apoE) levels in Alzheimer's disease. Neurosci. Lett. 223: 29-32.

Tamada K., Fujinaga S., Watanabe R., Yamashita R., Takeuchi Y., Osano M. (1995) Specific deposition of passively transferred monoclonal antibodies against herpes simplex virus type 1 in rat brain infected with the virus. Microbiol-Immunol. 39: 861-871.

The Lund and Manchester Groups. (1994) Clinical and neuropathological criteria for frontotemporal dementia. J. Neurol. Neurosurg. Psychiatry 57: 416-418.

Tumani H., Shen G., Peter J. B., Bruck W. (1999) Glutamine synthetase in cerebrospinal fluid, serum, and brain: a diagnostic marker for Alzheimer disease? Arch. Neurol. 56: 1241-1246.

Vanmechelen E., Vanderstichele H., Davidsson P., Van Kerschaver E., Van Der Perre B., Sjogren M., Andreasen N., Blennow K. (2000) Quantification of tau phosphorylated at threonine 181 in human cerebrospinal fluid: a sandwich ELISA with a synthetic phosphopeptide for standardization. Neurosci. Lett. 285: 49-52.

Wakabayashi T., Yoshida J., Okada H., Sugita K., Itoh K., Tadokoro M., Ohshima M. (1995) Radioimaging of human glioma by indium-11 labelled G-22 anti-glioma monoclonal antibody. Noshuyo-Byori 12: 105-110.

WHO. (1997) International Classification of Diseases, Tenth Revision (ICD-10).

Wild D. (ed.) (2001) The Immunoassay Handbook 2^{nd} edition. Nature Pr., London, UK.

Wilson J. D., Braunwald E., Isselbacher K. J., Petersdorf R. G., Martin J. B, Fauci A. S., Root R. K. (1991) Harrison's Principles of Internal Medicine, 12th Edition, McGraw-Hill Inc., NY, USA.

Wolozin B. (2001) A fluid connection: cholesterol and Abeta. Proc. Natl. Acad. Sci. 98: 5371-5373.

Yaffe K., Gregg E. W. (2002) Diabetes mellitus, cognitive function, and risk of dementia in older adults. Available at: http://www.harrisonsonline.com/server-java/Arknoid/amed/harrisons/ex_editorials/ed12566_p01.html. Accessed Apr. 26, 2002.

Yan J. X., Tonella L., Sanchez J. C., Wilkins M. R., Packer N. H., Gooley A. A., Hochstrasser D. F., Williams K. L. (1997) The Dictyostelium discoideum proteome—the SWISS-2DPAGE database of the multicellular aggregate (slug). Electrophoresis 18: 491-497.

Zerr I., Bodemer M., Otto M., Poser S., Windl O., Kretzschmar H. A., Gefeller O., Weber T. (1996) Diagnosis of Creutzfeldt-Jakob disease by two-dimensional gel electrophoresis of cerebrospinal fluid. Lancet 348: 846-849.

1. A method for the screening, (differential) diagnosis and/or prognosis in a mammal of Alzheimer's disease, frontotemporal dementia, dementia with Lewy bodies, vascular dementia and/or depression, for identifying a mammal at risk of developing Alzheimer's disease, frontotemporal dementia, dementia with Lewy bodies, vascular dementia and/or depression, or for monitoring the effect of therapy administered to a mammal having Alzheimer's disease, frontotemporal dementia, dementia with Lewy bodies, vascular dementia and/or depression, said method comprising the following steps:
(a) detecting, in said mammal, the level of at least one of the following proteins: Apo E, α-1-antitrypsin, α-1-β glycoprotein, antithrombin III, Apo A-I, Apo A-IV, Apo J, gelsolin, haptoglobin, hemopexin, Ig α-1 chain C region (heavy), kininogen, prostaglandin-H2 D-isomerase, transthyretin, vitamin D-binding protein, Zn-α-2-glycoprotein, or of an isoform thereof; and
(b) comparing the level of said at least one protein or protein isoform detected in step (a) with a range of levels previously defined as characteristic for mammals suffering from AD, with a range of levels previously defined as characteristic for mammals suffering from FTD, with a range of levels previously defined as characteristic for mammals suffering from DLB, with a range of levels previously defined as characteristic for mammals suffering from VAD, with a range of levels previously defined as characteristic for mammals suffering from depression and with a range of levels previously defined as characteristic for control mammals; and
(c) concluding from the comparison in step (b) whether the mammal is suffering from Alzheimer's disease, frontotemporal dementia, dementia with Lewy bodies, vascular dementia and/or depression, whereby a level of said at least one protein or protein isoform in a range previously defined as characteristic for mammals suffering from AD is an indication that said mammal is suffering from AD; and whereby a level of said at least one protein or protein isoform in a range previously defined as characteristic for mammals suffering from FTD is an indication that said mammal is suffering from FTD; and whereby a level of said at least one protein or protein isoform in a range previously defined as characteristic for mammals suffering from DLB is an indication that said mammal is suffering from DLB; and whereby a level of said at least one protein or protein isoform in a range previously defined as characteristic for mammals suffering from VAD is an indication that said mammal is suffering from VAD; and whereby a level of said at least one protein or protein isoform in a range previously defined as characteristic for mammals suffering from depression is an indication that said mammal is suffering from depression.

2. The method according to claim 1, for the screening, diagnosis and/or prognosis in a mammal of Alzheimer's disease, frontotemporal dementia, dementia with Lewy bodies, vascular dementia and/or depression, for identifying a mammal at risk of developing Alzheimer's disease, frontotemporal dementia, dementia with Lewy bodies, vascular dementia and/or depression, or for monitoring the effect of therapy administered to a mammal having Alzheimer's disease, frontotemporal dementia, dementia with Lewy bodies, vascular dementia and/or depression, said method comprising the following steps:
(a) detecting, in said mammal, the level of at least one of the following proteins: Apo E, α-1-antitrypsin, α-1-β glycoprotein, antithrombin III, Apo A-I, Apo A-IV, Apo J, gelsolin, haptoglobin, hemopexin, Ig α-1 chain C region (heavy), kininogen, prostaglandin-H2 D-isomerase, transthyretin, vitamin D-binding protein, Zn-α-2-glycoprotein, or of an isoform thereof; and
(b) comparing the level of said at least one protein or protein isoform detected in step (a) with the level of said at least one protein or protein isoform in a control mammal; and
(c) concluding from the comparison in step (b) whether the mammal is suffering from Alzheimer's disease, frontotemporal dementia, dementia with Lewy bodies, vascular dementia and/or depression, an altered level of said at least one protein or protein isoform compared to said level in a control mammal being an indication of the mammal suffering from Alzheimer's disease, frontotemporal dementia, dementia with Lewy bodies, vascular dementia and/or depression.

3. The method according to claim 1, for the differential diagnosis in a mammal of Alzheimer's disease, frontotemporal dementia, dementia with Lewy bodies, vascular dementia and/or depression, said method comprising the following steps:
(a) detecting, in said mammal, the level of at least one of the following proteins: Apo E, α-1-antitrypsin, α-1-β glycoprotein, antithrombin III, Apo A-I, Apo A-IV, Apo J, gelsolin, haptoglobin, hemopexin, Ig α-1 chain C region (heavy), kininogen, prostaglandin-H2 D-isomerase, transthyretin, vitamin D-binding protein, Zn-α-2-glycoprotein, or of an isoform thereof; and
(b) comparing the level of said at least one protein or protein isoform detected in step (a) with the level of said at least one protein or protein isoform in a mammal suffering from another neurological disease.
(c) concluding from the comparison in step (b) whether the mammal is suffering from Alzheimer's disease, frontotemporal dementia, dementia with Lewy bodies, vascular dementia and/or depression.

4. The method according to any of claims 1 to 3, further characterized that the level of at least one of the following neurological disease-associated protein isoforms is detected (Table 2; Table 3; Table 4; Table 6):
Apo E: NPI 11, NPI 34, NPI 35, NPI 41, NPI 52, NPI 60, NPI 66, NPI 72, NPI 73, NPI 74, NPI 75, NPI 76m, NPI 77;
α-1-antitrypsin: NPI 1, NPI 42, NPI 43, NPI 44, NPI 59;
α-1-β glycoprotein: NPI 2, NPI 3, NPI 31, NPI 48;
Antithrombin-III: NPI 4;
Apo A-I: NPI 5, NPI 6, NPI 7, NPI 37, NPI 69, NPI 70, NPI 71;
Apo A-IV: NPI 8, NPI 9, NPI 10;
Apo J: NPI 12, NPI 13, NPI 14, NPI 15, NPI 16;
Gelsolin: NPI 17;
Haptoglobin: NPI 18;
Hemopexin: NPI 19, NPI 20;
Ig α-1 chain C region (heavy): NPI 21, NPI 22;
Kininogen: NPI 23;
Prostaglandin-H2 D-isomerase: NPI 24, NPI 25;
Transthyretin: NPI 26, NPI 27, NPI 28m;
Vitamin D-binding protein: NPI 29, NPI 30;
Zn-α-2-glycoprotein: NPI 33;
NPI 32, NPI 36, NPI 38, NPI 39, NPI 40, NPI 45, NPI 46, NPI 47, NPI 49, NPI 50, NPI 51, NPI 53, NPI 54, NPI 55, NPI 56, NPI 57, NPI 58, NPI 61, NPI 62, NPI 63, NPI 64, NPI 65, NPI 67, NPI 68.

5. The method according to claim 2, for the screening, diagnosis or prognosis in a mammal of Alzheimer's disease, for identifying a mammal at risk of developing Alzheimer's disease, or for monitoring the effect of therapy administered to a mammal having Alzheimer's disease, said method comprising the following steps:
(a) detecting, in said mammal, the level of at least one of the following protein isoforms (Table 2): NPI 1, NPI 16, NPI 25;
(b) comparing the level of said at least one protein isoform detected in step (a) with the level of said at least one protein isoform in a control mammal; and
(c) concluding from the comparison in step (b) whether the mammal is suffering from Alzheimer's disease, a decreased level of said at least one protein isoform compared to the level of said at least one protein isoform in a control mammal being an indication of the mammal suffering from Alzheimer's disease.

6. The method according to claim 2, for the screening, diagnosis or prognosis in a mammal of frontotemporal dementia, for identifying a mammal at risk of developing frontotemporal dementia, or for monitoring the effect of therapy administered to a mammal having frontotemporal dementia, said method comprising the following steps:
(a) detecting, in said mammal, the level of at least one of the following protein isoforms (Table 2): NPI 5, NPI 6, NPI 12, NPI 17, NPI 24;
(b) comparing the level of said at least one protein isoform detected in step (a) with the level of said at least one protein isoform in a control mammal; and
(c) concluding from the comparison in step (b) whether the mammal is suffering from frontotemporal dementia, a decreased level of said at least one protein isoform compared to the level of said at least one protein isoform in a control mammal being an indication of the mammal suffering from frontotemporal dementia.

7. The method according to claim 2, for the screening, diagnosis or prognosis in a mammal of frontotemporal dementia, for identifying a mammal at risk of developing frontotemporal dementia, or for monitoring the effect of therapy administered to a mammal having frontotemporal dementia, said method comprising the following steps:
(a) detecting, in said mammal, the level of at least one of the following protein isoforms (Table 2): NPI 4, NPI 8, NPI 9, NPI 10, NPI 18, NPI 19, NPI 20, NPI 22, NPI 23, NPI 28m, NPI 70;
(b) comparing the level of said at least one protein isoform detected in step (a) with the level of said at least one protein isoform in a control mammal; and
(c) concluding from the comparison in step (b) whether the mammal is suffering from frontotemporal dementia, an increased level of said at least one protein isoform compared to the level of said at least one protein isoform in a control mammal being an indication of the mammal suffering from frontotemporal dementia.

8. The method according to claim 3, for the differential diagnosis in a mammal of Alzheimer's disease versus frontotemporal dementia, said method comprising the following steps:
(a) detecting, in said mammal, the level of at least one of the following protein isoforms (Table 2): NPI 5, NPI 6, NPI 26;
(b) comparing the level of said at least one protein isoform detected in step (a) with a previously defined cut-off value suitable for differentiating mammals suffering from AD versus mammals suffering from FTD; and
(c) concluding from the comparison in step (b) whether the mammal is suffering from AD or from FTD, whereby a level of said at least one protein isoform above the cut-off value being an indication of the mammal suffering from Alzheimer's disease; and whereby a level of said at least one protein isoform below the cut-off value being an indication of the mammal suffering from FTD.

9. The method according to claim 3, for the differential diagnosis in a mammal of Alzheimer's disease versus frontotemporal dementia, said method comprising the following steps:
(a) detecting, in said mammal, the level of at least one of the following protein isoforms (Table 2): NPI 2, NPI 3, NPI 7, NPI 8, NPI 9, NPI 11, NPI 13, NPI 14, NPI 15, NPI 16, NPI 21, NPI 22, NPI 25, NPI 27, NPI 28m, NPI 29, NPI 30, NPI 69, NPI 71;
(b) comparing the level of said at least one protein isoform detected in step (a) with a previously defined cut-off value suitable for differentiating mammals suffering from AD versus mammals suffering from FTD; and
(c) concluding from the comparison in step (b) whether the mammal is suffering from AD or from FTD, whereby a level of said at least one protein isoform below the cut-off value being an indication of the mammal suffering from Alzheimer's disease; and whereby a level of said at least one protein isoform above the cut-off value being an indication of said mammal suffering from FTD.

10. The method according to claim 3, for the differential diagnosis in a mammal of Alzheimer's disease versus depression, said method comprising the following steps:
(a) detecting, in said mammal, the level of at least one of the following protein isoforms (Table 2; Table 6): NPI 6, NPI 12, NPI 23, NPI 31, NPI 32, NPI 33, NPI 34, NPI 35, NPI 36, NPI 37, NPI 38, NPI 40, NPI 41, NPI 42, NPI 43, NPI 44, NPI 45, NPI 46, NPI 47, NPI 48, NPI 51, NPI 52, NPI 53, NPI 54, NPI 55, NPI 56, NPI 58, NPI 59, NPI 60, NPI 61, NPI 63, NPI 68, NPI 69;
(b) comparing the level of said at least one protein isoform detected in step (a) with a previously defined cut-off value suitable for differentiating mammals suffering from AD versus mammals suffering from depression; and
(c) concluding from the comparison in step (b) whether the mammal is suffering from Alzheimer's disease or from depression, whereby a level of said at least one protein isoform below the cut-off value being an indication of the mammal suffering from Alzheimer's disease; and whereby a level of said at least one protein isoform above the cut-off value being an indication of said mammal suffering from depression.

11. The method according to claim 3, for the differential diagnosis in a mammal of Alzheimer's disease versus depression, said method comprising the following steps:
(a) detecting, in said mammal, the level of at least one of the following protein isoforms (Table 2; Table 6): NPI 39, NPI 49, NPI 50, NPI 57, NPI 62, NPI 64, NPI 65, NPI 66, NPI 67;
(b) comparing the level of said at least one protein isoform detected in step (a) with a previously defined cut-off value suitable for differentiating mammals suffering from AD versus mammals suffering from depression; and
(c) concluding from the comparison in step (b) whether the mammal is suffering from Alzheimer's disease or from depression, whereby a level of said at least one protein isoform above the cut-off value being an indication of the mammal suffering from Alzheimer's disease; and whereby a level of said at least one protein isoform below the cut-off value being an indication of said mammal suffering from depression.

12. The method according to claim 3, for the differential diagnosis in a mammal of Alzheimer's disease versus vascular dementia, said method comprising the following steps:
(a) detecting, in said mammal, the level of at least one of the following protein isoforms (Table 2): NPI 7, NPI 74, NPI 76m;
(b) comparing the level of said at least one protein isoform detected in step (a) with a previously defined cut-off value suitable for the differentiating mammals suffering from AD versus mammals suffering from VAD; and
(c) concluding from the comparison in step (b) whether the mammal is suffering from Alzheimer's disease or from VAD, whereby a level of said at least one protein isoform below the cut-off value being an indication of the mammal suffering from Alzheimer's disease; and whereby a level of said at least one protein isoform above the cut-off value being an indication of said mammal suffering from VAD.

13. The method according to claim 3, for the differential diagnosis in a mammal of Alzheimer's disease versus vascular dementia, said method comprising the following steps:
(a) detecting, in said mammal, the level of the following protein isoform (Table 2): NPI 5;
(b) comparing the level of said protein isoform detected in step (a) with a previously defined cut-off value suitable for differentiating mammals suffering from AD versus mammals suffering from VAD; and
(c) concluding from the comparison in step (b) whether the mammal is suffering from Alzheimer's disease or from VAD, whereby a level of said protein isoform above the cut-off value being an indication of the mammal suffering from Alzheimer's disease; and whereby a level of said protein isoform below the cut-off value being an indication of said mammal suffering from VAD.

14. The method according to any of claims 1 to 13, further characterized that said method is carried out in vitro, on a sample obtained from said mammal.

15. The method according to claim 14, further characterized that said sample is taken from the cerebrospinal fluid, plasma or serum of said mammal.

16. The method according to any of claims 1 to 15, characterized in that the level of protein or protein isoform is detected by isoelectric focusing followed by sodium dodecyl sulfate polyacrylamide gel electrophoresis (SDS-PAGE).

17. The method according to any of claims 1 to 15, characterized in that the level of protein or protein isoform is detected by an immunoassay.

18. The method according to claim 17, further characterized that the detection of the level of protein or protein isoform comprises:
(a) contacting the protein or protein isoform with an antibody that specifically recognizes the protein or protein isoform under conditions being suitable for producing an antigen-antibody complex; and
(b) detecting the immunological binding that has occurred between the antibody and the protein or protein isoform.

19. A composition comprising at least one of the following isolated protein isoforms associated with Alzheimer's disease, frontotemporal dementia, dementia with Lewy bodies, vascular dementia and/or depression:
Apo E: NPI 11, NPI 34, NPI 35, NPI 41, NPI 52, NPI 60, NPI 66, NPI 72, NPI 73, NPI 74, NPI 75, NPI 76m, NPI 77;
α-1-antitrypsin: NPI 1, NPI 42, NPI 43, NPI 44, NPI 59;
α-1-β glycoprotein: NPI 2, NPI 3, NPI 31, NPI 48;
Antithrombin-III: NPI 4;
Apo A-I: NPI 5, NPI 6, NPI 7, NPI 37, NPI 69, NPI 70, NPI 71;
Apo A-IV: NPI 8, NPI 9, NPI 10;
Apo J: NPI 12, NPI 13, NPI 14, NPI 15, NPI 16;
Gelsolin: NPI 17;
Haptoglobin: NPI 18;
Hemopexin: NPI 19, NPI 20;
Ig α-1 chain C region (heavy): NPI 21, NPI 22;
Kininogen: NPI 23;
Prostaglandin-H2 D-isomerase: NPI 24, NPI 25;
Transthyretin: NPI 26, NPI 27, NPI 28m;
Vitamin D-binding protein: NPI 29, NPI 30;
Zn-α-2-glycoprotein: NPI 33;
NPI 32, NPI 36, NPI 38, NPI 39, NPI 40, NPI 45, NPI 46, NPI 47, NPI 49, NPI 50, NPI 51, NPI 53, NPI 54, NPI 55, NPI 56, NPI 57, NPI 58, NPI 61, NPI 62, NPI 63, NPI 64, NPI 65, NPI 67, NPI 68.

20. An antibody capable of specifically recognizing one of the following protein isoforms associated with Alzheimer's disease, frontotemporal dementia, dementia with Lewy bodies, vascular dementia and/or depression:
Apo E: NPI 11, NPI 34, NPI 35, NPI 41, NPI 52, NPI 60, NPI 66, NPI 72, NPI 73, NPI 74, NPI 75, NPI 76m, NPI 77;
α-1-antitrypsin: NPI 1, NPI 42, NPI 43, NPI 44, NPI 59;
α-1-β glycoprotein: NPI 2, NPI 3, NPI 31, NPI 48;
Antithrombin-III: NPI 4;
Apo A-I: NPI 5, NPI 6, NPI 7, NPI 37, NPI 69, NPI 70, NPI 71;
Apo A-IV: NPI 8, NPI 9, NPI 10;
Apo J: NPI 12, NPI 13, NPI 14, NPI 15, NPI 16;
Gelsolin: NPI 17;
Haptoglobin: NPI 18;
Hemopexin: NPI 19, NPI 20;
Ig α-1 chain C region (heavy): NPI 21, NPI 22;
Kininogen: NPI 23;
Prostaglandin-H2 D-isomerase: NPI 24, NPI 25;
Transthyretin: NPI 26, NPI 27, NPI 28m;
Vitamin D-binding protein: NPI 29, NPI 30;
Zn-α-2-glycoprotein: NPI 33;
NPI 32, NPI 36, NPI 38, NPI 39, NPI 40, NPI 45, NPI 46, NPI 47, NPI 49, NPI 50, NPI 51, NPI 53, NPI 54, NPI 55, NPI 56, NPI 57, NPI 58, NPI 61, NPI 62, NPI 63, NPI 64, NPI 65, NPI 67, NPI 68.

21. A kit comprising an antibody according to claim 20.

22. An antibody according to claim 20 or a kit according to claim 21 for use in the screening, diagnosis or prognosis in a mammal of Alzheimer's disease, frontotemporal dementia, dementia with Lewy bodies, vascular dementia and/or depression, for identifying a mammal at risk of developing Alzheimer's disease, frontotemporal dementia, dementia with Lewy bodies, vascular dementia and/or depression, or for monitoring the effect of therapy administered to a mammal having Alzheimer's disease, frontotemporal dementia, dementia with Lewy bodies, vascular dementia and/or depression.

23. An antibody according to claim 20 or a kit according to claim 21 for use in the differential diagnosis in a mammal of Alzheimer's disease, frontotemporal dementia, dementia with Lewy bodies, vascular dementia and/or depression.

24. Use of an antibody according to claim 20 for the preparation of a kit for the screening, diagnosis or prognosis in a mammal of Alzheimer's disease, frontotemporal dementia, dementia with Lewy bodies, vascular dementia and/or depression, for identifying a mammal at risk of developing Alzheimer's disease, frontotemporal dementia, dementia with Lewy bodies, vascular dementia and/or depression, or for monitoring the effect of therapy administered to a mammal having Alzheimer's disease, frontotemporal dementia, dementia with Lewy bodies, vascular dementia and/or depression.

25. Use of an antibody according to claim 20 for the preparation of a kit for the differential diagnosis in a mammal of Alzheimer's disease, frontotemporal dementia, dementia with Lewy bodies, vascular dementia and/or of depression.

26. A method of screening for agents that interact with and/or modulate the expression or activity of a protein or protein isoform according to claim 16, said method comprising:
(a) contacting said protein or protein isoform or a portion of said protein or protein isoform with said agent; and
(b) determining whether or not said agent interacts with and/or modulates the expression of said protein or protein isoform or said portion of the protein or protein isoform.


SEQUENCE LISTING (113 sequences)

SEQ ID NO: 1 - protein, 9 aa, homo sapiens
LQAEAFQAR

SEQ ID NO: 2 - protein, 11 aa, homo sapiens
AKLEEQAQQIR

SEQ ID NO: 3 - protein, 10 aa, homo sapiens
LLRDADDLQK

SEQ ID NO: 4 - protein, 15 aa, homo sapiens
KVEQAVETEPEPELR

SEQ ID NO: 5 - protein, 9 aa, homo sapiens
LGPLVEQGR

SEQ ID NO: 6 - protein, 17 aa, homo sapiens
VQAAVGTSAAPVPSDNH

SEQ ID NO: 7 - protein, 15 aa, homo sapiens
AATVGSLAGQPLQER

SEQ ID NO: 8 - protein, 15 aa, homo sapiens
GEVQAMLGQSTEELR

SEQ ID NO: 9 - protein, 15 aa, homo sapiens
SELEEQLTPVAEETR

SEQ ID NO: 10 - protein, 11 aa, homo sapiens
ALMDETMKELK

SEQ ID NO: 11 - protein, 18 aa, homo sapiens
AYKSELEEQLTPVAEETR

SEQ ID NO: 12 - protein, 11 aa, homo sapiens
LSKELQAAQAR

SEQ ID NO: 13 - protein, 15 aa, homo sapiens
TDTSHHDQDHPTFNK

SEQ ID NO: 14 - protein, 10 aa, homo sapiens
LVDKFLEDVK

SEQ ID NO: 15 - protein, 7 aa, homo sapiens
FLEDVKK

SEQ ID NO: 16 - protein, 9 aa, homo sapiens
KQINDYVEK

SEQ ID NO: 17 - protein, 8 aa, homo sapiens
QINDYVEK

SEQ ID NO: 18 - protein, 16 aa, homo sapiens
DTEEEDFHVDQATTVK

SEQ ID NO: 19 - protein, 16 aa, homo sapiens
DTEEEDFHVDQVTTVK

SEQ ID NO: 20 - protein, 15 aa, homo sapiens
LQHLENELTHDIITK

SEQ ID NO: 21 - protein, 7 aa, homo sapiens
FLENEDR

SEQ ID NO: 22 - protein, 8 aa, homo sapiens
FLENEDRR

SEQ ID NO: 23 - protein, 8 aa, homo sapiens
SASLHLPK

SEQ ID NO: 24 - protein, 10 aa, homo sapiens
LSITGTYDLK

SEQ ID NO: 25 - protein, 10 aa, homo sapiens
SVLGQLGITK

SEQ ID NO: 26 - protein, 18 aa, homo sapiens
VFSNGADLSGVTEEAPLK

SEQ ID NO: 27 - protein, 8 aa, homo sapiens
AVLTIDEK

SEQ ID NO: 28 - protein, 9 aa, homo sapiens
LSSWVLLMK

SEQ ID NO: 29 - protein, 10 aa, homo sapiens
LGMFNIQHCK

SEQ ID NO: 30 - protein, 8 aa, homo sapiens
LLELTGPK

SEQ ID NO: 31 - protein, 11 aa, homo sapiens
ATWSGAVLAGR

SEQ ID NO: 32 - protein, 8 aa, homo sapiens
FALVREDR

SEQ ID NO: 33 - protein, 9 aa, homo sapiens
CLAPLEGAR

SEQ ID NO: 34 - protein, 10 aa, homo sapiens
LETPDFQLFK

SEQ ID NO: 35 - protein, 12 aa, homo sapiens
EVPLNTIIFMGR

SEQ ID NO: 36 - protein, 13 aa, homo sapiens
DYVSQFEGSALGK

SEQ ID NO: 37 - protein, 10 aa, homo sapiens
VQPYLDDFQK

SEQ ID NO: 38 - protein, 5 aa, homo sapiens
ALKED

SEQ ID NO: 39 - protein, 11 aa, homo sapiens
ATEHLSTLSEK

SEQ ID NO: 40 - protein, 9 aa, homo sapiens
AKPALEDLR

SEQ ID NO: 41 - protein, 9 aa, homo sapiens
LSPLGEEMR

SEQ ID NO: 42 - protein, 11 aa, homo sapiens
THLAPYSDELR

SEQ ID NO: 43 - protein, 14 aa, homo sapiens
LLDNWDSVTSTFSK

SEQ ID NO: 44 - protein, 17 aa, homo sapiens
DSGRDYVSQFEGSALGK

SEQ ID NO: 45 - protein, 11 aa, homo sapiens
LEALKENGGAR

SEQ ID NO: 46 - protein, 11 aa, homo sapiens
QGLLPVLESFK

SEQ ID NO: 47 - protein, 13 aa, homo sapiens
VEPLRAELQEGAR

SEQ ID NO: 48 - protein, 11 aa, homo sapiens
LSPLGEEMRDR

SEQ ID NO: 49 - protein, 12 aa, homo sapiens
DNDDSVTSTFSK

SEQ ID NO: 50 - protein, 11 aa, homo sapiens
QEMSKDLEEVK

SEQ ID NO: 51 - protein, 11 aa, homo sapiens
VQPYLDDFQKK

SEQ ID NO: 52 - protein, 7 aa, homo sapiens
LEALKED

SEQ ID NO: 53 - protein, 11 aa, homo sapiens
DLATVYVDVLK

SEQ ID NO: 54 - protein, 9 aa, homo sapiens
QKLHELQEK

SEQ ID NO: 55 - protein, 10 aa, homo sapiens
DEPPQSPWDR

SEQ ID NO: 56 - protein, 12 aa, homo sapiens
VSFLSALEEYTK

SEQ ID NO: 57 - protein, 10 aa, homo sapiens
KWQEEMELYR

SEQ ID NO: 58 - protein, 13 aa, homo sapiens
LGEVNTYAGDLQK

SEQ ID NO: 59 - protein, 11 aa, homo sapiens
LLPHANEVSQK

SEQ ID NO: 60 - protein, 8 aa, homo sapiens
QLTPYAQR

SEQ ID NO: 61 - protein, 11 aa, homo sapiens
IDQNVEELKGR

SEQ ID NO: 62 - protein, 9 aa, homo sapiens
LTPYADEFK

SEQ ID NO: 63 - protein, 9 aa, homo sapiens
ISASAEELR

SEQ ID NO: 64 - protein, 9 aa, homo sapiens
LAPLAEDVR

SEQ ID NO: 65 - protein, 10 aa, homo sapiens
ALVQQMEQLR

SEQ ID NO: 66 - protein, 9 aa, homo sapiens
LEPYADQLR

SEQ ID NO: 67 - protein, 9 aa, homo sapiens
IDQTVEELR

SEQ ID NO: 68 - protein, 11 aa, homo sapiens
RVEPYGENFNK

SEQ ID NO: 69 - protein, 12 aa, homo sapiens
SLAPYAQDTQEK

SEQ ID NO: 70 - protein, 11 aa, homo sapiens
ALMDETMKELK

SEQ ID NO: 71 - protein, 11 aa, homo sapiens
ELDESLQVAER

SEQ ID NO: 72 - protein, 7 aa, homo sapiens
KYNELLK

SEQ ID NO: 73 - protein, 8 aa, homo sapiens
FMETVAEK

SEQ ID NO: 74 - protein, 16 aa, homo sapiens
EILSVDCSTNNPSQAK

SEQ ID NO: 75 - protein, 10 aa, homo sapiens
TLLSNLEEAK

SEQ ID NO: 76 - protein, 9 aa, homo sapiens
IDSLLENDR

SEQ ID NO: 77 - protein, 12 aa, homo sapiens
ASSIIDELFQDR

SEQ ID NO: 78 - protein, 13 aa, homo sapiens
AGALNSNDAFVLK

SEQ ID NO: 79 - protein, 9 aa, homo sapiens
YIETDPANR

SEQ ID NO: 80 - protein, 8 aa, homo sapiens
TGAQELLR

SEQ ID NO: 81 - protein, 13 aa, homo sapiens
TEGDGVYTLNDKK

SEQ ID NO: 82 - protein, 18 aa, homo sapiens
TEGDGVYTLNDKKQWINK

SEQ ID NO: 83 - protein, 11 aa, homo sapiens
NFPSPVDAAFR

SEQ ID NO: 84 - protein, 11 aa, homo sapiens
GGYTLVSGYPK

SEQ ID NO: 85 - protein, 10 aa, homo sapiens
QGHNSVFLIK

SEQ ID NO: 86 - protein, 9 aa, homo sapiens
DYFMPCPGR

SEQ ID NO: 87 - protein, 9 aa, homo sapiens
SAVQGPPER

SEQ ID NO: 88 - protein, 17 aa, homo sapiens
QEPSQGTTTFAVTSILR

SEQ ID NO: 89 - protein, 9 aa, homo sapiens
TPLTATLSK

SEQ ID NO: 90 - protein, 11 aa, homo sapiens
TVGSDTFYSFK

SEQ ID NO: 91 - protein, 9 aa, homo sapiens
QVVAGLNFR

SEQ ID NO: 92 - protein, 8 aa, homo sapiens
YFIDFVAR

SEQ ID NO: 93 - protein, 16 aa, homo sapiens
APEAQVSVQPNFQQDK

SEQ ID NO: 94 - protein, 16 aa, homo sapiens
TMLLQPAGSLGSYSYR

SEQ ID NO: 95 - protein, 17 aa, homo sapiens
AQGFTEDTIVFLPQTDK

SEQ ID NO: 96 - protein, 10 aa, homo sapiens
EAQVSVQPNF

SEQ ID NO: 97 - protein, 13 aa, homo sapiens
AADDTDEPFASGK

SEQ ID NO: 98 - protein, 5 aa, homo sapiens
PLMVK

SEQ ID NO: 99 - protein, 15 aa, homo sapiens
GPTGTGESKCPLMVK

SEQ ID NO: 100 - protein, 13 aa, homo sapiens
AADDTWEPFASGK

SEQ ID NO: 101 - protein, 17 aa, homo sapiens
QDPPSVVVTSHQAPGEK

SEQ ID NO: 102 - protein, 14 aa, homo sapiens
KAADDTWEPFASGK

SEQ ID NO: 103 - protein, 22 aa, homo sapiens
TSESGELHGLTTEEEFVEGIYK

SEQ ID NO: 104 - protein, 11 aa, homo sapiens
HLSLLTTLSNR

SEQ ID NO: 105 - protein, 7 aa, homo sapiens
YTFELSR

SEQ ID NO: 106 - protein, 10 aa, homo sapiens
THLPEVFLSK

SEQ ID NO: 107 - protein, 7 aa, homo sapiens
VLEPTLK

SEQ ID NO: 108 - protein, 8 aa, homo sapiens
ELSSFIDK

SEQ ID NO: 109 - protein, 11 aa, homo sapiens
VCSQYAAYGEK

SEQ ID NO: 110 - protein, 11 aa, homo sapiens
VMDKYTFELSR

SEQ ID NO: 111 - protein, 14 aa, homo sapiens
CCDVEDSTTCFNAK

SEQ ID NO: 112 - protein, 9 aa, homo sapiens
AKLPDATPK

SEQ ID NO: 113 - protein, 10 aa, homo sapiens
AGEVQEPELR